(19) United States
(12) Patent Application Publication
(10) Pub. No.: US 2026/0317389 A1
(43) Pub. Date: Oct. 15, 2026

(54) ENGINEERED IMMUNOLOGICAL CELLS

(71) Applicants: TCRCURE BIOPHARMA CORP., Durham, NC (US); GUANGDONG TCRCURE BIOPHARMA TECHNOLOGY CO., LTD., Guangzhou (CN)

(72) Inventors: Lifeng Zhang, Singapore (SG); Lang Dou, Guangzhou (CN); Si Li, Alhambra, CA (US); Junjie Feng, Durham, NC (US); Zhenbo Su, Durham, NC (US)

(21) Appl. No.: 18/998,331

(22) PCT Filed: Jul. 25, 2023
(86) PCT No.: PCT/CN2023/109081

§ 371 Date: Jan. 24, 2025

Related U.S. Application Data
(60) Provisional application No. 63/392,016, filed on Jul. 25, 2022.

(51) Int. Cl. C07K 14/54; A61K 35/17; A61K 40/15; A61K 40/31; A61K 40/42; A61P 35/00; C07K 14/715; C07K 16/28; C12N 5/0783

(52) U.S. Cl. CPC C07K 14/5443; A61K 35/17; A61K 40/15; A61K 40/31; A61K 40/4217; A61K 40/4224; A61P 35/00; C07K 14/7155; C07K 16/2827; C07K 16/2866; C12N 5/0646; A61K 2239/13; A61K 2239/29; C07K 2317/14; C07K 2317/52; C07K 2317/565; C07K 2317/622; C07K 2317/732; C12N 2510/00

(57) ABSTRACT

The disclosure is related to engineered immune cells (e.g., cord blood-derived NK cells) expressing a cell membrane-bound IL15 that can promote autonomous growth (e.g., in the absence of supporting cytokines) and persistence. In some embodiments, the immune cells can also express and/or secret an antibody or peptibody that can specifically bind to an antigen/receptor on target cells (e.g., cancer cells), which can be eliminated via Fc-mediated ADCC/ADCP/CDC activities.

### CLAIM OF PRIORITY

This application claims the benefit of U.S. Provisional Application App. No. 63/392,016, filed on Jul. 25, 2022. The entire contents of the foregoing application are incorporated herein by reference.

### BACKGROUND

The seminal clinical success of using autologous CAR-T cells targeting CD19 on B cell surface in the treatment of B-cell related tumors opened the curtain of a new era of gene and cell based drug design. CAR-T cells carry an artificial protein called “chimeric antigen receptor” (CAR), which is engineered by combining the antigen-recognition domain of a selected antibody for target recognition and selected intracellular signaling domains for T cell activation. CAR is introduced into T cells to grant the cells the capacity of recognizing a pre-selected cancer target. Because of immune rejection, without further manipulation, T-cell based therapies are typically autologous, meaning that the drug cells are not sold as off-the-shelf drugs, rather the therapeutic service is performed, one case at a time, in a costly, slow, tedious and inefficient manner. Moreover, it is difficult to maintain quality consistency among individual samples. This is a pressing issue faced by the current CAR-T technology.

Natural killer (NK) cells are innate lymphocytes that are cytotoxic and suitable for allogeneic usage, and a large body of evidence from the literatures and the clinical trials has demonstrated the safety of NK cells in allogeneic usage. The druggability of NK cells has been demonstrated in Liu E et al. Use of CAR-Transduced Natural Killer Cells in CD19-Positive Lymphoid Tumors. N Engl J Med 2020; 382:545-553, where the cord blood derived CAR-NK cells against CD19 showed potent clinical efficacy in treating B-cell related tumors. Cases of complete remission (CR) were observed even at drug dosages as low as 1×105 cells per kg body weight. Although the long-term clinical outcome is still waiting to be confirmed, the data show that the clinical efficacy of CAR-NK cells is comparable to CAR-T cells at least in short term. Meanwhile, CAR-NK showed lower level of adverse events in the treatment compared to CAR-T.

NK cells are effector cells of antibody-dependent cellular cytotoxicity (ADCC). CD16, a cell surface protein of mature NK cells, functions as an Fc receptor, which recognizes the fragment crystallizable (Fc) region of antibodies and enables NK cells to recognize target cells through the guidance of antibodies. In contrast, T cells do not have ADCC capacity.

Compared to T cells which form immune memory and, therefore, are able to persist for long term after infusion, NK cells are not capable of forming strong memory-like immune responses and therefore cannot survive for long term after infusion. Several cytokines, e.g., interleukins such as IL-2, IL-15, IL-12, IL-21, and IL-18, have been used to expand primary NK cells in vitro. However, the use of these cytokines has the following shortfalls. Firstly, the half-life for these cytokines is typically very short. Secondly, most, if not all, of these cytokines requires an indirect and seemingly circumlocutory mechanism for signal presentation to function. These shortfalls necessitate the frequent reapplication of these cytokines when culturing and/or expanding, thereby causing the quality inconsistency to negatively influence the druggability of the NK cells cultured that way.

### SUMMARY

The present disclosure provides engineered immune cells (e.g., NK cells) capable of autonomous growth and having prolonged persistence in vitro, such that the cells can serve as an ideal off-the-shelf and/or allogeneic cell drug platform. In some embodiments, the immune cells can express an engineered cell membrane-bound IL15, obtained by fusing the cytokine ligand IL15 with a membrane-bound portion (e.g., IL15 receptor, alpha subunit (i.e., IL15Rα)). On the cell membrane of the immune cells, the engineered membrane-bound IL15 can directly activate the downstream cytokine signaling pathway even in the absence of the cytokine supplement(s) (e.g., IL-2, IL-15, etc.), which may be realized due to the capability of the engineered membrane-bound IL15-IL15Rα to form a functional complex with the other two subunits of the IL15 signaling complex, including the beta subunit (“IL15Rβ”, which is substantially the beta subunit of IL-2 receptor or “IL2Rβ”; and the two terms IL15Rβ and IL2Rβ are deemed exchangeable throughout the disclosure) and the gamma subunit (IL15Rγ, which is substantially the gamma subunit of IL-2 receptor or IL2Rγ or γC; and the terms IL15Rγ, IL2Rγ, and γC are deemed exchangeable throughout the disclosure).

The present disclosure also provides engineered immune cells (e.g., NK cells) that express and/or secrete one or more Fc-containing antibodies and/or Fc-containing peptibodies that can selectively recognize and bind to corresponding cell-surface target molecules that are specifically expressed on one or more target cells (e.g., cancer cells). In some embodiments, the cells can express an anti-PD-L1 antibody or antigen-binding fragment thereof (e.g., scFv), or a ligand polypeptide that specifically binds to IL13Rα2. When secreted, such molecules can label the target cells and recruit the immune cells (e.g., NK cells) to specifically eliminate target cells (e.g., cancer cells) via ADCC, ADCP, and/or CDC activities.

In one aspect, the disclosure is related to an engineered immune cell, expressing a chimeric polypeptide comprising an interleukin 15 (IL15) domain and an interleukin 15 receptor, alpha subunit (IL15Rα) domain. In some embodiments, when expressed, the chimeric polypeptide is cell membrane-bound, and when a population of the engineered immune cell is cultured in vitro, the total cell number thereof remains increased or substantially unchanged after at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, or at least 35 days of culture without any supporting cytokines.

As used herein, the “supporting cytokines” or “supporting cytokine supplements”, can include any of IL-2, IL-15, IL-12, IL-21, and IL-18, or any of their combinations.

As used herein, the phrase “remain substantially unchanged” means that compared with the total cell number (N₀) on the reference Day 0 of culture, the total cell number (Nᵢ) at Day i (i>0) of culture is within 10% of variance of NO, i.e., Nᵢ is within 90%-110% of N₀; the phrase “remain increased”, “remain higher”, “remain elevated” or alike, means that Nᵢ is >110% of N₀; and the phrase “remain decreased”, “remain lower” or alike, means that Nᵢ is <90% of N₀.

In some embodiments, when expressed, the chimeric polypeptide is able to operatively form a functional complex with interleukin 15 receptor, beta subunit (IL15Rβ, which is substantially IL2Rβ) and interleukin 15 receptor, gamma subunit (IL15Rγ, which is substantially IL2Rγ or γC) to thereby be able to activate the downstream γC signaling in the engineered immune cell in the absence of IL-2 or IL-15.

In some embodiments, the immune cell is a natural killer (NK) cell, a T cell, or a tumor-infiltrating cell (TIL), preferably a NK cell. In some embodiments, the immune cell is an NK cells. In some embodiments, the engineered immune cell is capable of autonomous growth when cultured in vitro. In some embodiments, the IL15 domain has an amino acid sequence that is at least 90%, at least 95%, at least 96%, at least 97%, at least 98%, or at least 99% identical to SEQ ID NO: 2. In some embodiments, the IL15 domain comprises an Asp residue at a position corresponding to Asn72 of SEQ ID NO: 2. In some embodiments, the IL15 domain has an amino acid sequence that is at least 90%, at least 95%, at least 96%, at least 97%, at least 98%, or at least 99% identical to SEQ ID NO: 3. In some embodiments, the IL15Rα domain has an amino acid sequence that is at least 90%, at least 95%, at least 96%, at least 97%, at least 98%, or at least 99% identical to SEQ ID NO: 5. In some embodiments, the IL15 domain and the IL15a domain are fused via an engineered linker, in some embodiments, the engineered linker comprises an amino acid sequence that is at least 90%, at least 95%, at least 96%, at least 97%, at least 98%, or at least 99% identical to SEQ ID NO: 4. In some embodiments, the chimeric polypeptide comprises an amino acid sequence that is at least 90%, at least 95%, at least 96%, at least 97%, at least 98%, or at least 99% identical to SEQ ID NO: 6 or SEQ ID NO: 7. In some embodiments, when a population of the immune cell is cultured in vitro, the total cell number thereof can remain substantially unchanged after at least 35 days of culture without any supporting cytokines. In some embodiments, the NK cell is obtained from cord blood.

In some embodiments, the engineered immune cell can further express and/or secret one or more polypeptides, each comprising a target-binding domain, in some embodiments, the target-binding domain is capable of specifically recognizing and binding to a target molecule expressed or presented on the cell surface of a target cell. In some embodiments, each of the one or more polypeptides further comprises an Fc domain, in some embodiments, the Fc domain is capable of mediating antibody-dependent cellular cytotoxicity (ADCC) for the engineered immune cell. In some embodiments, the Fc domain comprises at least one of Ser239Asp, Ala330Leu, Ile332Glu, Phe243Leu, Arg292Pro, Tyr300Leu, Val305Ile, Pro396Leu, Met428Leu, or Asn434Ser according to EU numbering. In some embodiments, the Fc domain comprises at least one set of: (a) Ser239Asp, Ala330Leu and Ile332Glu; (b) Phe243Leu, Arg292Pro, Tyr300Leu, Val305Ile and Pro396Leu; or (c) Met428Leu and Asn434Ser. In some embodiments, the Fc domain comprises a combination of Ser239Asp, Ala330Leu, Ile332Glu, Phe243Leu, Arg292Pro, Tyr300Leu, Val305Ile, Pro396Leu, Met428Leu and Asn434Ser.

In some embodiments, each of the one or more polypeptides further comprises a first masking peptide fused at the N-terminal of the antigen-binding domain or the C-terminal of the Fc domain via a first flexible linker, in some embodiments, the first masking peptide is configured to block the Fc domain of the polypeptide, and the first flexible linker is configured to be cleavable in a target tissue. In some embodiments, the target tissue is a tumor tissue, and the first flexible linker is configured to be degradable by matrix metalloproteinase 1 (MMP1), matrix metalloproteinase 2 (MMP2), matrix metalloproteinase 3 (MMP3), matrix metalloproteinase 7 (MMP7), matrix metalloproteinase 9 (MMP9), and/or matrix metalloproteinase 14 (MMP14). In some embodiments, the first flexible linker is configured to be degradable by MMP9, and the first flexible linker has an amino acid sequence selected from SEQ ID NO: 26 or SEQ ID NO: 27. In some embodiments, the first flexible linker is configured to be degradable by a proteinase (protease) that is exogenously provided to the target tissue. In some embodiments, each of the one or more polypeptides further comprises a second masking peptide fused at the N-terminal of the antigen-binding domain or the C-terminal of the Fc domain via a second flexible linker, in some embodiments, the second masking peptide is configured to block the antigen-binding domain of the polypeptide and the second flexible linker is configured to be cleavable in a target tissue. In some embodiments, the target tissue is a tumor tissue, and the second flexible linker is configured to be degradable by MMP1, MMP2, MMP3, MMP7, MMP9 or MMP14. In some embodiments, the second flexible linker is configured to be degradable by MMP9, and the second flexible linker has an amino acid sequence selected from SEQ ID NO: 26 or SEQ ID NO: 27. In some embodiments, the second flexible linker is configured to be degradable by a proteinase (protease) that is exogenously provided to the target tissue.

In some embodiments, the one or more polypeptides comprises an antibody or antigen-binding fragment thereof, in some embodiments, the target-binding domain is an antigen-binding domain, comprising a single-chain variable fragment (scFv) domain or a single monomeric variable antibody domain. In some embodiments, the target molecule is selected from a group consisting of PD-L1, BCMA, Caudin18.2, CCR4, CD10, CD123, CD147, CD171, CD19, CD20, CD22, CD276, CD319, CD33, CD38, CD70, CLL-1, DLL3, EGFR, EGFRVIII, EpCAM, FLT3, FRα, GD2, GPC3, HER2, HGFR, IL13Ralpha2, Mesothelin, MG7, MUC1, MUC16, Nectin4, PSCA, ROR1, ROR2, TACI, TRBC1, TSLPR, and VEGFR. In some embodiments, the antibody or antigen-binding fragment thereof is an anti-PD-L1 antibody or antigen-binding fragment thereof. In some embodiments, the anti-PD-L1 antibody or antigen-binding fragment thereof comprises an scFv domain, in some embodiments, the scFv domain comprises a VL domain having an amino acid sequence that is at least 90%, at least 95%, at least 96%, at least 97%, at least 98%, or at least 99% identical to SEQ ID NO: 9, and/or comprises a VH domain having an amino acid sequence that is at least 90%, at least 95%, at least 96%, at least 97%, at least 98%, or at least 99% identical to SEQ ID NO: 10. In some embodiments, the scFv domain of the anti-PD-L1 antibody or antigen-binding fragment thereof comprises an amino acid sequence that is at least 90%, at least 95%, at least 96%, at least 97%, at least 98%, or at least 99% identical to SEQ ID NO: 12. In some embodiments, the anti-PD-L1 antibody or antigen-binding fragment thereof comprises an scFv domain, in some embodiments, the scFv domain comprises a VL domain having an amino acid sequence that is at least 90%, at least 95%, at least 96%, at least 97%, at least 98%, or at least 99% identical to SEQ ID NO: 13, and/or comprises a VH domain having an amino acid sequence that is at least 90%, at least 95%, at least 96%, at least 97%, at least 98%, or at least 99% identical to SEQ ID NO: 14. In some embodiments, the scFv domain of the anti-PD-L1 antibody or antigen-binding fragment thereof has an amino acid sequence that is at least 90%, at least 95%, at least 96%, at least 97%, at least 98%, or at least 99% identical to SEQ ID NO: 15.

In some embodiments, the one or more polypeptides comprises a peptibody, in some embodiments, the target-binding domain comprises a ligand polypeptide. In some embodiments, the target molecule is a receptor that the ligand polypeptide is capable of specifically recognizing and binding, in some embodiments, the receptor is selected from a group consisting of IL13Rα2, APN/CD13, APP, PD-L1, CD44, P32/gC1qR, E-cadherin, N-cadherin, CD21, EGFR, Epha2, EphB4, HER2, FGFR1, FGFR2, FGFR3, FGFR4, VEGFR1, VEGFR3, PSMA, GPC3, IL-10RA, IL-11Rα, IL-6Rα, GP130, VEGFR2, MUC18, Met, MMP9, Thomsen-Friedenreich carbohydrate antigen, NRP-1, PDGFRβ, CD133, PTPRJ, HSPG, E-selectin, Tie2, VPAC1, ActRIIB, CD163, CXCR4, Ephrin A4, Ephrin B1, Ephrin B2, Ephrin B3, Gonadotrophin releasing hormone receptor, G Protein-coupled receptor 55, Bombesin receptor 2, IL4 receptor, Low-density lipoprotein receptor, Leptin receptor, LRP1, Melanocortin 1 receptor, Melanocortin 4 receptor, CD206, Urokinase plasminogen activator receptor, Neurokinin-1 receptor, VPAC2, ITGB1, CD27, ITGB5, ITGA1, CD27, LRP1, ACVR2B, COL13A1, NOTCH3, EGFR, VEGFR2, VEGFR3, PDGFR, HER2, ErbB3, ErbB4, RET, and FGFR102. In some embodiments, the peptibody is an anti-IL13Rα2 peptibody, in some embodiments, the ligand polypeptide thereof comprises an amino acid sequence that is at least 90%, at least 95%, at least 96%, at least 97%, at least 98%, or at least 99% identical to SEQ ID NO: 21. In some embodiments, the ligand polypeptide comprises one or more substitutions corresponding to positions 11, 90, 107, 103, and 108-111 of SEQ ID NO: 21. In some embodiments, the ligand polypeptide comprises a Tyr residue at a position corresponding to Glu11 of SEQ ID NO: 21. In some embodiments, the ligand polypeptide comprises an amino acid sequence that is at least 90%, at least 95%, at least 96%, at least 97%, at least 98%, or at least 99% identical to SEQ ID NO: 22. In some embodiments, the ligand polypeptide comprises a ligand-mimicking peptide and/or an artificial peptide.

In one aspect, the disclosure is related to a pharmaceutical composition for treating a cancer in a subject in need thereof, comprising: a therapeutical effective number of the engineered immune cell as described herein; and a pharmaceutically acceptable adjuvant.

In one aspect, the disclosure is related to a method for treating a cancer in a subject in need thereof, comprising: administering a therapeutically effective number of the engineered immune cell as described herein to the subject.

In one aspect, the disclosure is related to a method for obtaining an autonomous growth of immune cells cultured in vitro, comprising: transducing into the immune cells a transgene encoding a chimeric polypeptide comprising an interleukin 15 (IL15) domain and an interleukin 15 receptor, alpha subunit (IL15Rα) domain, such that when expressed in the immune cells, the chimeric polypeptide is cell membrane-bound and operatively forms a functional complex with interleukin 15 receptor, beta subunit (IL15Rβ) and interleukin 15 receptor, gamma subunit (IL15RY), in some embodiments, the transduced immune cells are featured such that the total cell number thereof can remain higher or substantially unchanged after at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, or at least 35 days of culture without any supporting cytokines. In some embodiments, the immune cell is a natural killer (NK) cell, a T cell, or a tumor-infiltrating cell (TIL). In some embodiments, the immune cell is an NK cell. In some embodiments, the IL15 domain has an amino acid sequence that is at least 90%, at least 95%, at least 96%, at least 97%, at least 98%, or at least 99% identical to SEQ ID NO: 2. In some embodiments, the IL15 domain comprises an Asp residue at a position corresponding to Asn72 of SEQ ID NO: 2. In some embodiments, the IL15 domain has an amino acid sequence that is at least 90%, at least 95%, at least 96%, at least 97%, at least 98%, or at least 99% identical to SEQ ID NO: 3. In some embodiments, the IL15Rα domain has an amino acid sequence that is at least 90%, at least 95%, at least 96%, at least 97%, at least 98%, or at least 99% identical to SEQ ID NO: 5. In some embodiments, the IL15 domain and the IL15a domain are fused via an engineered linker, in some embodiments, the engineered linker has an amino acid sequence that is at least 90%, at least 95%, at least 96%, at least 97%, at least 98%, or at least 99% identical to SEQ ID NO: 4. In some embodiments, the chimeric polypeptide has an amino acid sequence that is at least 90%, at least 95%, at least 96%, at least 97%, at least 98%, or at least 99% identical to SEQ ID NO: 6 or SEQ ID NO: 7. In some embodiments, upon transduction, the total cell number of the transduced NK cells remains higher or substantially unchanged after at least 35 days of culture without any supporting cytokines. In some embodiments, the NK cell is obtained from cord blood.

In one aspect, the disclosure is related to an engineered immune cell, expressing and/or secreting one or more polypeptides, each comprising a target-binding domain, in some embodiments, the target-binding domain is capable of specifically recognizing and binding to a target molecule expressed or presented on the cell surface of a target cell. In some embodiments, the immune cell is a natural killer (NK) cell, a T cell, or a tumor-infiltrating cell (TIL). In some embodiments, the immune cell is an NK cell. In some embodiments, each of the one or more polypeptides further comprises an Fc domain, in some embodiments, the Fc domain is capable of mediating antibody-dependent cellular cytotoxicity (ADCC) for the engineered immune cell. In some embodiments, the Fc domain comprises at least one of Ser239Asp, Ala330Leu, Ile332Glu, Phe243Leu, Arg292Pro, Tyr300Leu, Val305Ile, Pro396Leu, Met428Leu, or Asn434Ser according to EU numbering. In some embodiments, the Fc domain comprises at least one set of: (a) Ser239Asp, Ala330Leu and Ile332Glu; (b) Phe243Leu, Arg292Pro, Tyr300Leu, Val305Ile and Pro396Leu; or (c) Met428Leu and Asn434Ser. In some embodiments, the Fc domain comprises a combination of Ser239Asp, Ala330Leu, Ile332Glu, Phe243Leu, Arg292Pro, Tyr300Leu, Val305Ile, Pro396Leu, Met428Leu and Asn434Ser. In some embodiments, each of the one or more polypeptides further comprises a first masking peptide fused at the N-terminal of the antigen-binding domain or the C-terminal of the Fc domain via a first flexible linker, in some embodiments, the first masking peptide is configured to block the Fc domain of the polypeptide, and the first flexible linker is configured to be cleavable in a target tissue. In some embodiments, the first flexible linker is configured to be degradable by a proteinase (protease) that is specifically or enrichingly expressed in the target tissue. In some embodiments, the proteinase is MMP1, MMP2, MMP3, MMP7, MMP9 or MMP14. In some embodiments, the proteinase is MMP9, and the first flexible linker has an amino acid sequence selected from SEQ ID NO: 26 or SEQ ID NO: 27. In some embodiments, the first flexible linker is configured to be degradable by a proteinase (protease) that is exogenously provided to the target tissue. In some embodiments, each of the one or more polypeptides further comprises a second masking peptide fused at the N-terminal of the antigen-binding domain or the C-terminal of the Fc domain via a second flexible linker, in some embodiments, the second masking peptide is configured to block the antigen-binding domain of the polypeptide and the second flexible linker is configured to be cleavable in a target tissue. In some embodiments, the second flexible linker is configured to be degradable by a proteinase (protease) that is specifically or enrichingly expressed in the target tissue. In some embodiments, the proteinase (protease) is MMP1, MMP2, MMP3, MMP7, MMP9 or MMP14. In some embodiments, the proteinase (protease) is MMP9, and the second flexible linker has an amino acid sequence selected from SEQ ID NO: 26 or SEQ ID NO: 27. In some embodiments, the second flexible linker is configured to be degradable by a proteinase (protease) that is exogenously provided to the target tissue.

In some embodiments, the one or more polypeptides comprises an antibody or antigen-binding fragment thereof, in some embodiments, the target-binding domain is an antigen-binding domain, comprising a single-chain variable fragment (scFv) domain or a single monomeric variable antibody domain. In some embodiments, the target molecule is selected from a group consisting of PD-L1, BCMA, Caudin18.2, CCR4, CD10, CD123, CD147, CD171, CD19, CD20, CD22, CD276, CD319, CD33, CD38, CD70, CLL-1, DLL3, EGFR, EGFRVIII, EpCAM, FLT3, FRα, GD2, GPC3, HER2, HGFR, IL13Ralpha2, Mesothelin, MG7, MUC1, MUC16, Nectin4, PSCA, ROR1, ROR2, TACI, TRBC1, TSLPR, and VEGFR. In some embodiments, the antibody or antigen-binding fragment thereof is an anti-PD-L1 antibody or antigen-binding fragment thereof. In some embodiments, the anti-PD-L1 antibody or antigen-binding fragment thereof comprises an scFv domain, in some embodiments, the scFv domain comprises a VL domain having an amino acid sequence that is at least 90%, at least 95%, at least 96%, at least 97%, at least 98%, or at least 99% identical to SEQ ID NO: 9, and/or comprises a VH domain having an amino acid sequence that is at least 90%, at least 95%, at least 96%, at least 97%, at least 98%, or at least 99% identical to SEQ ID NO: 10. In some embodiments, the scFv domain of the anti-PD-L1 antibody or antigen-binding fragment thereof has an amino acid sequence that is at least 90%, at least 95%, at least 96%, at least 97%, at least 98%, or at least 99% identical to SEQ ID NO: 12. In some embodiments, the anti-PD-L1 antibody or antigen-binding fragment thereof comprises an scFv domain, in some embodiments, the scFv domain comprises a VL domain having an amino acid sequence that is at least 90%, at least 95%, at least 96%, at least 97%, at least 98%, or at least 99% identical to SEQ ID NO: 13, and/or comprises a VH domain having an amino acid sequence that is at least 90%, at least 95%, at least 96%, at least 97%, at least 98%, or at least 99% identical to SEQ ID NO: 14. In some embodiments, the scFv domain of the anti-PD-L1 antibody or antigen-binding fragment thereof has an amino acid sequence that is at least 90%, at least 95%, at least 96%, at least 97%, at least 98%, or at least 99% identical to SEQ ID NO: 15.

In some embodiments, the one or more polypeptides comprises a peptibody, in some embodiments, the target-binding domain comprises a ligand polypeptide. In some embodiments, the target molecule is a receptor that the ligand polypeptide is capable of specifically recognizing and binding, in some embodiments, the receptor is selected from a group consisting of IL13Rα2, APN/CD13, APP, PD-L1, CD44, P32/gC1qR, E-cadherin, N-cadherin, CD21, EGFR, Epha2, EphB4, HER2, FGFR1, FGFR2, FGFR3, FGFR4, VEGFR1, VEGFR3, PSMA, GPC3, IL-10RA, IL-11Rα, IL-6Rα, GP130, VEGFR2, MUC18, Met, MMP9, Thomsen-Friedenreich carbohydrate antigen, NRP-1, PDGFRβ, CD133, PTPRJ, HSPG, E-selectin, Tie2, VPAC1, ActRIIB, CD163, CXCR4, Ephrin A4, Ephrin B1, Ephrin B2, Ephrin B3, Gonadotrophin releasing hormone receptor, G Protein-coupled receptor 55, Bombesin receptor 2, IL4 receptor, Low-density lipoprotein receptor, Leptin receptor, LRP1, Melanocortin 1 receptor, Melanocortin 4 receptor, CD206, Urokinase plasminogen activator receptor, Neurokinin-1 receptor, VPAC2, ITGB1, CD27, ITGB5, ITGA1, CD27, LRP1, ACVR2B, COL13A1, NOTCH3, EGFR, VEGFR2, VEGFR3, PDGFR, HER2, ErbB3, ErbB4, RET, and FGFR102. In some embodiments, the peptibody is an anti-IL13Rα2 peptibody, in some embodiments, the ligand polypeptide thereof comprises an amino acid sequence that is at least 90%, at least 95%, at least 96%, at least 97%, at least 98%, or at least 99% identical to SEQ ID NO: 21. In some embodiments, the ligand polypeptide comprises a one or more substitutions corresponding to positions 11, 90, 107, 103, and 108-111 of SEQ ID NO: 21. In some embodiments, the ligand polypeptide comprises a Tyr residue at a position corresponding to Glu11 of SEQ ID NO: 21. In some embodiments, the ligand polypeptide comprises an amino acid sequence that is at least 90%, at least 95%, at least 96%, at least 97%, at least 98%, or at least 99% identical to SEQ ID NO: 22. In some embodiments, the ligand polypeptide comprises a ligand-mimicking peptide and/or an artificial peptide. In some embodiments, the engineered NK cell is capable of autonomous growth when cultured in vitro.

In some embodiments, the cell further expresses a chimeric polypeptide comprising an interleukin 15 (IL15) domain and an interleukin 15 receptor, alpha subunit (IL15Rα) domain, in some embodiments, when expressed, the chimeric polypeptide is cell membrane-bound. In some embodiments, when a population of the NK cells are cultured in vitro, the total cell number thereof can remain higher or substantially unchanged after at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, or at least 35 days of culture without any supporting cytokines. In some embodiments, when expressed, the chimeric polypeptide is able to operatively form a functional complex with IL15Rβ/IL2Rβ and IL15Rγ/IL2Rγ/γC to activate the downstream cytokine signaling in the engineered NK cell. In some embodiments, the engineered NK cells are obtained from cord blood.

In one aspect, the disclosure is related to a pharmaceutical composition for treating a cancer in a subject in need thereof, comprising: a therapeutical effective number of the engineered immune cell as described herein; and a pharmaceutically acceptable adjuvant.

In one aspect, the disclosure is related to a method for treating a cancer in a subject in need thereof, comprising: administering a therapeutically effective number of the engineered immune cell as described herein to the subject.

In one aspect, the disclosure is related to an antibody or antigen-binding fragment thereof that binds to PD-L1, comprising an antigen-binding domain that specifically binds to PD-L1, in some embodiments, the antigen-binding domain comprises an scFv domain, in some embodiments, the scFv domain comprises a VL domain having an amino acid sequence that is at least 90%, at least 95%, at least 96%, at least 97%, at least 98%, or at least 99% identical to SEQ ID NO: 9, and/or comprises a VH domain having an amino acid sequence that is at least 90%, at least 95%, at least 96%, at least 97%, at least 98%, or at least 99% identical to SEQ ID NO: 10. In some embodiments, the scFv domain of the anti-PD-L1 antibody has an amino acid sequence that is at least 90%, at least 95%, at least 96%, at least 97%, at least 98%, or at least 99% identical to SEQ ID NO: 12.

In one aspect, the disclosure is related to an antibody or antigen-binding fragment thereof that binds to PD-L1, comprising an antigen-binding domain that specifically binds to PD-L1, in some embodiments, the antigen-binding domain comprises an scFv domain, in some embodiments, the scFv domain comprises a VL domain having an amino acid sequence that is at least 90%, at least 95%, at least 96%, at least 97%, at least 98%, or at least 99% identical to SEQ ID NO: 13, and/or comprises a VH domain having an amino acid sequence that is at least 90%, at least 95%, at least 96%, at least 97%, at least 98%, or at least 99% identical to SEQ ID NO: 14. In some embodiments, the scFv domain of the anti-PD-L1 antibody has an amino acid sequence that is at least 90%, at least 95%, at least 96%, at least 97%, at least 98%, or at least 99% identical to SEQ ID NO: 15. In some embodiments, the antibody or antigen-binding fragment thereof described herein further comprises an Fc domain, in some embodiments, the Fc domain is capable of mediating antibody-dependent cellular cytotoxicity (ADCC). In some embodiments, the Fc domain comprises at least one of Ser239Asp, Ala330Leu, Ile332Glu, Phe243Leu, Arg292Pro, Tyr300Leu, Val305Ile, Pro396Leu, Met428Leu, or Asn434Ser according to EU numbering. In some embodiments, the Fc domain comprises at least one set of: (a) Ser239Asp, Ala330Leu and Ile332Glu; (b) Phe243Leu, Arg292Pro, Tyr300Leu, Val305Ile and Pro396Leu; or (c) Met428Leu and Asn434Ser. In some embodiments, the Fc domain comprises a combination of Ser239Asp, Ala330Leu, Ile332Glu, Phe243Leu, Arg292Pro, Tyr300Leu, Val305Ile, Pro396Leu, Met428Leu and Asn434Ser.

In one aspect, the disclosure is related to an antibody or antigen-binding fragment thereof that binds to programed death-ligand 1 (PD-L1), comprising: a heavy chain variable region (VH) comprising complementarity determining regions (CDRs) 1, 2, and 3, in some embodiments, the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and a light chain variable region (VL) comprising CDRs 1, 2, and 3, in some embodiments, the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence, in some embodiments, the selected VH CDRs 1, 2, and 3 amino acid sequences and the selected VL CDRs, 1, 2, and 3 amino acid sequences are one of the following: (1) the selected VH CDRs 1, 2, 3 amino acid sequences are set forth in SEQ ID NOs: 31, 32, 33, respectively, and the selected VL CDRs 1, 2, 3 amino acid sequences are set forth in SEQ ID NOs: 34, 35, 36, respectively; (2) the selected VH CDRs 1, 2, 3 amino acid sequences are set forth in SEQ ID NOs: 37, 38, 39, respectively, and the selected VL CDRs 1, 2, 3 amino acid sequences are set forth in SEQ ID NOs: 40, 41, 42, respectively; (3) the selected VH CDRs 1, 2, 3 amino acid sequences are set forth in SEQ ID NOs: 43, 44, 45, respectively, and the selected VL CDRs 1, 2, 3 amino acid sequences are set forth in SEQ ID NOs: 46, 47, 48, respectively; and (4) the selected VH CDRs 1, 2, 3 amino acid sequences are set forth in SEQ ID NOs: 49, 50, 51, respectively, and the selected VL CDRs 1, 2, 3 amino acid sequences are set forth in SEQ ID NOs: 52, 53, 54, respectively. In some embodiments, the VH comprises CDRs 1, 2, 3 with the amino acid sequences set forth in SEQ ID NOs: 31, 32, and 33, respectively, and the VL comprises CDRs 1, 2, 3 with the amino acid sequences set forth in SEQ ID NOs: 34, 35, and 36, respectively according to Kabat definition. In some embodiments, the VH comprises CDRs 1, 2, 3 with the amino acid sequences set forth in SEQ ID NOs: 37, 38, and 39, respectively, and the VL comprises CDRs 1, 2, 3 with the amino acid sequences set forth in SEQ ID NOs: 40, 41, and 42, respectively according to Kabat definition. In some embodiments, the VH comprises CDRs 1, 2, 3 with the amino acid sequences set forth in SEQ ID NOs: 43, 44, and 45, respectively, and the VL comprises CDRs 1, 2, 3 with the amino acid sequences set forth in SEQ ID NOs: 46, 47, and 48, respectively according to North/Aho definition. In some embodiments, the VH comprises CDRs 1, 2, 3 with the amino acid sequences set forth in SEQ ID NOs: 49, 50, and 51, respectively, and the VL comprises CDRs 1, 2, 3 with the amino acid sequences set forth in SEQ ID NOs: 52, 53, and 54, respectively according to North/Aho definition. In some embodiments, the antibody or antigen-binding fragment specifically binds to human PD-L1. In some embodiments, the antibody or antigen-binding fragment is a human or humanized antibody or antigen-binding fragment thereof. In some embodiments, the antibody or antigen-binding fragment is a single-chain variable fragment (scFv) or a multi-specific antibody (e.g., a bispecific antibody).

In one aspect, the disclosure is related to a nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 31, 32, and 33, respectively (or SEQ ID NOs: 43, 44, and 45, respectively), and in some embodiments, the VH, when paired with a light chain variable region (VL) comprising the amino acid sequence set forth in SEQ ID NO: 13 binds to PD-L1;
(2) an immunoglobulin light chain or a fragment thereof comprising a VL comprising CDRs 1, 2, and 3 comprising the amino acid sequences set forth in SEQ ID NOs: 34, 35, and 36, respectively (or SEQ ID NOs: 46, 47, and 48, respectively), and in some embodiments, the VL, when paired with a VH comprising the amino acid sequence set forth in SEQ ID NO: 14 binds to PD-L1;
(3) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 37, 38, and 39, respectively (or SEQ ID NOs: 49, 50, and 51, respectively), and in some embodiments, the VH, when paired with a light chain variable region (VL) comprising the amino acid sequence set forth in SEQ ID NO: 9 binds to PD-L1; or
(4) an immunoglobulin light chain or a fragment thereof comprising a VL comprising CDRs 1, 2, and 3 comprising the amino acid sequences set forth in SEQ ID NOs: 40, 41, and 42, respectively (or SEQ ID NOs: 52, 53, and 54, respectively), and in some embodiments, the VL, when paired with a VH comprising the amino acid sequence set forth in SEQ ID NO: 10 binds to PD-L1.

In some embodiments, the VH when paired with a VL specifically binds to human PD-L1, or the VL when paired with a VH specifically binds to human PD-L1. In some embodiments, the immunoglobulin heavy chain or the fragment thereof is a human or humanized immunoglobulin heavy chain or a fragment thereof, and the immunoglobulin light chain or the fragment thereof is a human or humanized immunoglobulin light chain or a fragment thereof. In some embodiments, the nucleic acid encodes a single-chain variable fragment (scFv), a multi-specific antibody (e.g., a bispecific antibody), or a chimeric antigen receptor (CAR). In some embodiments, the nucleic acid is cDNA.

In one aspect, the disclosure is related to a vector comprising one or more of the nucleic acids as described herein. In one aspect, the disclosure is related to a vector comprising two of the nucleic acids as described herein, in some embodiments, the vector encodes the VH region and the VL region that together bind to PD-L1. In one aspect, the disclosure is related to a pair of vectors, in some embodiments, each vector comprises one of the nucleic acids as described herein, in some embodiments, together the pair of vectors encodes the VH region and the VL region that together bind to PD-L1.

In one aspect, the disclosure is related to a cell comprising the vector, or the pair of vectors as described herein. In some embodiments, the cell is a CHO cell. In one aspect, the disclosure is related to a cell comprising one or more of the nucleic acids as described herein.

In one aspect, the disclosure is related to a method of producing an antibody or an antigen-binding fragment thereof, the method comprising (a) culturing the cell as described herein under conditions sufficient for the cell to produce the antibody or the antigen-binding fragment; and (b) collecting the antibody or the antigen-binding fragment produced by the cell.

In one aspect, the disclosure is related to an antibody or antigen-binding fragment thereof that binds to PD-L1 comprising a heavy chain variable region (VH) comprising an amino acid sequence that is at least 80% identical to a selected VH sequence, and a light chain variable region (VL) comprising an amino acid sequence that is at least 80% identical to a selected VL sequence, in some embodiments, the selected VH sequence and the selected VL sequence are one of the following: (1) the selected VH sequence is SEQ ID NO: 14, and the selected VL sequence is SEQ ID NO: 13; and (2) the selected VH sequence is SEQ ID NO: 10, and the selected VL sequence is SEQ ID NO: 9. In some embodiments, the antibody or antigen-binding fragment specifically binds to human PD-L1. In some embodiments, the antibody or antigen-binding fragment is a human or humanized antibody or antigen-binding fragment thereof. In some embodiments, the antibody or antigen-binding fragment is a single-chain variable fragment (scFv) or a multi-specific antibody (e.g., a bispecific antibody).

In one aspect, the disclosure is related to an antibody or antigen-binding fragment thereof that cross-competes with the antibody or antigen-binding fragment thereof as described herein.

In one aspect, the disclosure is related to an antibody or antigen-binding fragment thereof that binds to PD-L1 comprising a heavy chain variable region (VH) comprising VH CDR1, VH CDR2, and VH CDR3 that are identical to VH CDR1, VH CDR2, and VH CDR3 of a selected VH sequence; and a light chain variable region (VL) comprising VL CDR1, VL CDR2, and VL CDR3 that are identical to VL CDR1, VL CDR2, and VL CDR3 of a selected VL sequence, in some embodiments, the selected VH sequence and the selected VL sequence are one of the following: (1) the selected VH sequence is SEQ ID NO: 14, and the selected VL sequence is SEQ ID NO: 13; and (2) the selected VH sequence is SEQ ID NO: 10, and the selected VL sequence is SEQ ID NO: 9.

In one aspect, the disclosure is related to an antibody-drug conjugate comprising the antibody or antigen-binding fragment thereof as described herein covalently bound to a therapeutic agent. In some embodiments, the therapeutic agent is a cytotoxic or cytostatic agent.

In one aspect, the disclosure is related to a method of treating a subject having cancer, the method comprising administering a therapeutically effective amount of a composition comprising the antibody or antigen-binding fragment thereof, or the antibody-drug conjugate as described herein, to the subject. In some embodiments, the subject has breast cancer, ovarian cancer, lung cancer, bladder cancer, prostate cancer, pancreatic cancer, gastric cancer, liver cancer, leukemia and/or lymphoma.

In one aspect, the disclosure is related to a method of decreasing the rate of tumor growth, the method comprising contacting a tumor cell with an effective amount of a composition comprising an antibody or antigen-binding fragment thereof, or the antibody-drug conjugate as described herein. In one aspect, the disclosure is related to a method of killing a tumor cell, the method comprising contacting a tumor cell with an effective amount of a composition comprising the antibody or antigen-binding fragment thereof, or the antibody-drug conjugate as described herein.

In one aspect, the disclosure is related to a pharmaceutical composition comprising the antibody or antigen-binding fragment thereof as described herein, and a pharmaceutically acceptable carrier. In one aspect, the disclosure is related to a pharmaceutical composition comprising the antibody drug conjugate as described herein, and a pharmaceutically acceptable carrier.

Also provided herein is a fusion protein comprising an amino acid sequence that is at least 80%, 85%, 90%, 95%, 96%, 97%, 98%, 99% or 100% identical to SEQ ID NO: 6, 7, 19, 20, or 25.

The disclosure also relates to methods of treating a subject having cancer, the method comprising administering a therapeutically effective number of the engineered immune cells (e.g., any of the engineered immune cells described herein) to the subject. The disclosure also relates to methods of decreasing the rate of tumor growth, the method comprising administering a therapeutically effective number of the engineered immune cells (e.g., any of the engineered immune cells described herein) to the subject. The disclosure also relates to methods of killing a tumor cells, the method comprising administering a therapeutically effective number of the engineered immune cells (e.g., any of the engineered immune cells described herein) to the subject. In some embodiments, the subject has a cancer cell expressing PD-L1 and/or IL13Rα2 on the cell surface. In some embodiments, the cancer is breast cancer, ovarian cancer, lung cancer, bladder cancer, prostate cancer, pancreatic cancer, gastric cancer, liver cancer, leukemia and lymphoma.

As used herein, the term “chimeric protein” or “chimeric polypeptide” refers to a protein or a polypeptide, which has at least one portion of the sequence that is derived from two or more different sources. In some embodiments, the chimeric protein or chimeric polypeptide described herein is a fusion protein or a fusion polypeptide.

As used herein, the term “drug cell” refers to engineered cells (e.g., any of the engineered immune cells) that can be used for treating diseases (e.g., cancers).

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1 is a schematic diagram of the construct structure and corresponding amino acid sequences for Recast-IL15 according to some embodiments of the disclosure. The Recast-IL15 construct comprises, from N-terminus to C-terminus, a signal peptide, the IL-15 domain, a linker region, and the IL-15Rα (or IL-15RA) domain.

FIG. 2 shows a schematic diagram of “Recast-IL15” and its working mechanism to sustain autonomous growth of NK cells. Recast-IL15 is an engineered protein comprising a membrane-bound IL15Rα fused with IL15. Compared with traditional working mechanism of the cytokine IL15, this design has advantages such as an increased in vitro and in vivo duration of IL15 (i.e., “long half-life”), being autonomous (i.e., “not replying on IL15-presenting cells), and substantially having IL15 working as an autocrine to sustain the growth of host NK cells (i.e., “by the cells, for the cells”).

FIGS. 3A-3B show schematic diagrams of the structure of two embodiments of an antibody expressed in and secreted by the NK cells that are capable of recognizing target antigens on target cells and are capable of ADCC/ADCP/CDC.

FIG. 4 shows the engineered Fc region of an antibody/antibody-like polypeptide carried by Ab-NK. The three sets of engineered amino acids along the Fc region of the antibody or antibody-like polypeptide carried by Ab-NK is shown separately. The amino acids are annotated according to the consensus sequence of the IgG1 Fc region with the starting amino acid Glu216 and ending amino acid Lys447 according to EU numbering.

FIGS. 5A-5B show structure and sequence of the full-length self-secreting PD-L1 antibody. (A) Construct design of the anti-PD-L1 antibody, which includes a signal peptide “IL2SP,” an antigen-binding domain “PD-L1 scFv,” and the “Fc (design)” domain whose sequence is engineered according to the embodiments described herein for prolonged half-life and enhanced ADCC, ADCP and CDC activity. It fulfills two types of functionality against cancer cells: immune checkpoint inhibitor and antibody-mediated immune cell responses. This design aims to maximize the therapeutic capacity of both the PD-L1 antibody and NK cells. (B) The structure of the full-length anti-PD-L1 antibody is shown together with its amino acid sequence.

FIG. 6 shows the design of Pluck-NK cells according to some embodiments of the disclosure, which are cord blood-derived NK cells equipped with two transgenic components: Recast-IL15 and a self-secreting full-length anti-PD-L1 antibody carrying an engineered Fc region.

FIG. 7 shows the mechanism of action (MOA) of Pluck-NK. Pluck-NK cells that are designed to stimulate multiple layers of immune responses to combat cancer cells, including antibody-dependent cellular cytotoxicity (ADCC), immune checkpoint blockade (ICB), antibody-dependent cellular phagocytosis (ADCP) and complement-dependent cytotoxicity (CDC).

FIGS. 8A-8B show schematic diagrams of the structure of two embodiments of an antibody expressed in and secreted by the NK cells that are capable of recognizing target antigens on target cells and are capable of ADCC/ADCP/CDC.

FIGS. 9A-9B show cell expansion results in culture. (A) The cell growth curve of two cord blood samples. (B) The final cell expansion fold (Day 14) of 25 cord blood samples.

FIGS. 10A-10B show the purity and transduction efficiency of the NK cell product on Day 14.

FIGS. 11A-11B show CD107A and IFNγ profiles, respectively, upon NK cell activation. CB-NK cells are NK cells derived from the cord blood without being engineered by any transgenes. K562 cells do not express MHC class I antigen. H441 (lung cancer) and ES2 (ovarian cancer) are two cell lines expressing PD-L1. To provide direct evidence of ADCC, cell culture medium conditioned by Pluck-NK cells were used in the experiment. The conditioned medium only contained the “supernatant” and the cell secretion, if any. The cells cultured in the medium were removed.

FIG. 12 shows cell killing assay results. The cells cultured in the medium were removed. E:T ratio is the ratio of effector cells to target cells.

FIG. 13 shows CD16 blocking assay results. To confirm that Pluck-NKs can eliminate tumor cells through CD16-mediated ADCC, pluck NK cells (“NK”) were pre-incubated with a CD16 blocking antibody (clone: B73.1; BioLegend Cat #: 360702) before performing the killing assay against PD-L1+ES-2 cell line.

FIG. 14A shows growth curves of Pluck-NK cells. CB-NK cells are NK cells derived from the cord blood without being engineered by transgenes (“NT NK”), and were used as control. Pluck-NK carried Recast-IL15.

FIG. 14B shows growth curves of CAR-NKs. This figure is directly copied from the publication of our benchmark technology platform (Liu, E., et al. “Cord blood NK cells engineered to express IL-15 and a CD19-targeted CAR show long-term persistence and potent antitumor activity.” Leukemia 32.2 (2018): 520-531; or “Doi: 10.1038/leu.2017.226”), where NT-NKs are non-transduced NK cells, and CAR-NKs are transduced NK cells carrying a wildtype self-secreting IL15 transgene.

FIG. 15 shows the anti-PD-L1 antibody production at 24 h, 48 h and 72 h after seeding. The concentration of the secreted antibody was measured by ELISA.

FIG. 16A shows representative flow cytometry results of fresh NK cells and cryopreserved NK cells. Cryopreservation-induced apoptosis (Annexin-V⁺ 7-AAD⁻ population) was observed in a fraction of pluck-NK cells.

FIG. 16B shows the percentage of apoptotic cells in fresh and cryopreserved pluck-NK cells. Collective data (n=3) were used to calculate the average and error bars.

FIG. 16C shows the percentage of IL15RA⁻ and IL15RA⁺ in the apoptotic NK cell population. IL-15RA⁻ indicate NK cells without transgenes. IL-15RA⁺ indicate NK cells with transgenes.

FIG. 16D shows the 24-hour recovery rate of all NK cells (left) and IL-15RA⁺ NK cells (right). NK cells from three different donors (NK1, NK2, and NK3) were used.

FIGS. 17A-17B show in vivo drug efficacy and safety of Pluck-NK cells. (A) In vivo efficacy of Pluck-NK measured by tumor growth. (B) In vivo safety of Pluck-NK monitored by mouse body weight measurements. Each group had 5 mice.

FIGS. 18A-18B show peptibody-NK activity in vitro. To assess peptibody-NK's efficacy in vitro, the cytotoxicity assay was performed. (A) The transduction efficacy of peptibody-NK. CB-NK (“CBNK”) indicates un-transduced NK cells. IL13-Fc NK indicates peptibody-NK cells. (B) Cytotoxicity against IL13Rα2⁺ glioblastoma cell line U251 at different effector: target ratios.

FIG. 19A shows cellular binding assay results of anti-PD-L1 antibodies.

FIG. 19B shows on-plate ELISA binding assay results of anti-PD-L1 antibodies.

FIG. 19C shows cellular blocking assay results of anti-PD-L1 antibodies.

FIG. 20A shows screening results of anti-PD-L1 TCRC-L8 using a Membrane Proteome Array.

FIG. 20B shows validation results of anti-PD-L1 TCRC-L8 by titration experiments.

FIG. 21 lists Kabat CDR sequences for anti-PD-L1 antibodies.

FIG. 22 lists North/Aho CDR sequences for anti-PD-L1 antibodies.

### DETAILED DESCRIPTION

The present disclosure has two aims. The first is to develop an off-the-shelf and/or allogeneic NK cell drug platform that is optimized for autonomous growth in vitro and prolonged persistence in vivo. The second is to establish an NK cell drug platform, which can fully utilize NK cells' capacity in fighting cancer, and can be customized for treating a wide variety of cancer types.

### Autonomously Growing NK Cell Drug Platform

Interleukin-15 (IL-15 or IL15) is 14-15 kDa glycoprotein encoded by the 34 kb region of chromosome 4q31 in humans, and at the central region of chromosome 8 in mice. Although IL-15 mRNA can be found in many cells and tissues including mast cells, cancer cells or fibroblasts, this cytokine is produced as a mature protein mainly by dendritic cells, monocytes and macrophages.

IL15 is a 4-a-helix bundle cytokine playing a pivotal role in stimulation of both innate and adaptive immune cells. IL15 induces the activation, the proliferation, and the survival of T cells and contributes to generation and maintenance of high-avidity, antigen-specific CD8+ memory T cells in the long term. In addition, IL15 is involved in the development, the persistence, and the activation of NK and NKT as well as γ/δ T cells.

The IL15 receptor (IL15R) is composed of three different molecules, better known as the α (CD215; unique to the IL15R), the β (CD122), and the γ (CD132) chains. In particular, CD122 is also a component of the IL2R, whereas CD132, also known as the common γ chain (γ_{c}), is shared with different cytokines, including IL2, IL4, IL7, IL9, and IL21. While the IL15Rβγ complex is present on target cells, IL15Rα can be expressed as a membrane-bound complex with IL15 on the surface of many cell types, including activated monocytes, dendritic cells (DC), and endothelial cells. Such a heterodimer is presented in trans to neighboring α/β, γ/δ T or NK cells. Alternatively, it can be shed and released as a soluble factor. It was indicated that virtually all circulating IL15 in human and mouse serum is complexed with IL15Rα. Triggering of the receptor activates downstream signaling pathways that include JAK1 and JAK3 as well as STAT3 and STAT5, followed by the recruitment of the PI3K/AKT/mTOR and RAS/RAF/MAPK-ERK cascades. By inducing FOS/JUN, MYC, NF-κB, and BCL2 genes expression and by decreasing the expression of BIM and PUMA, IL15 has a stimulating effect on T-cell proliferation and survival.

Because sharing the β and γ components of the receptor, IL2 and IL15 exert similar functions on T cells. Indeed, both stimulate the proliferation of T cells, facilitate the differentiation of cytotoxic T lymphocytes (CTL), and induce the generation and maintenance of NK cells. Nevertheless, mice deficient in IL2 or IL15 have different phenotypes, and administration of IL2 and IL15 to mice, primates, or humans leads to distinct effects on cells of the immune system. As regards to antigen-activated effector cells, while IL2 promotes terminal differentiation and, eventually, their elimination by activation-induced cell death (AICD), IL15 inhibits AICD and promotes the generation of long-lived memory T cells as well as their maintenance by homeostatic proliferation.

IL15 and its IL15Rα chain are coexpressed by monocytes/macrophages and dendritic cells and subsequently displayed as a cell surface IL15: IL15Rα complex, which is trans-presented to neighboring immune cells expressing IL2Rβγc. Therefore, IL15 does not support maintenance of Tregs. Rather than inducing apoptosis of activated CD8+ T cells, IL15 provides anti-apoptotic signals. IL15 also has non-redundant roles in the development, proliferation, and activation of NK cells. IL15 does not induce significant capillary leak syndrome in mice or nonhuman primates (NHP), suggesting that IL15-based therapies may provide the immunostimulatory benefits of IL2 with fewer adverse effects.

Interleukin 15 receptor, alpha subunit (CD215), also known as IL15RA or IL15Rα, is a subunit of the interleukin 15 receptor that in humans is encoded by the IL15RA gene. The IL-15 receptor is composed of three subunits: IL-15R alpha, CD122, and CD132. Two of these subunits, CD122 and CD132, are shared with the receptor for IL-2, but IL-2 receptor has an additional subunit (CD25). The shared subunits contain the cytoplasmic motifs required for signal transduction, and this forms the basis of many overlapping biological activities of IL15 and IL2, although in vivo the two cytokines have separate biological effects. IL-15R alpha specifically binds IL15 with very high affinity, and is capable of binding IL-15 independently of other subunits. It is suggested that this property allows IL-15 to be produced by one cell, endocytosed by another cell, and then presented to a third party cell. This receptor is reported to enhance cell proliferation and expression of apoptosis inhibitor BCL2L1/BCL2-XL and BCL2. Multiple alternatively spliced transcript variants of this gene have been reported.

IL-15Rα can be expressed on the surface of T or NK cells, forming an IL-15Rα/IL-2Rβ/γc trimeric receptor. However, IL-15Rα appears to be mainly expressed by antigen-presenting cells. It binds IL-15 with a high affinity, allowing a producing cell to present IL-15 in trans via IL-15Rα to a neighboring cell that expresses the IL-2Rβ/γc complex. This original mechanism of action is called IL-15 trans-presentation. Thus, IL-15 can act both in cis, like IL-2, but also in trans. In addition, a soluble(s) form of IL-15Rα (sIL-15Rα) can act either as an antagonist of IL-15 action, competing with membrane-bound IL-15Rα for the binding of IL-15 or, as an agonist, forming an IL-15Rα/IL-15 complex activating the IL-2Rβ/γc dimeric receptor more efficiently than IL-15 alone. Thus, numerous laboratories turned the latter observation into therapeutic applications to mimic trans-presentation of soluble IL-15Rα/IL-15 (sIL-15Rα/IL-15, also referred to as ‘trans-signaling’ of IL-15Rα/IL-15).

A detailed description of IL15, IL15RA, and their functions can be found, e.g., in Pilipow K., et al. “IL15 and T-cell Stemness in T-cell-Based Cancer Immunotherapy.” Cancer Research 75.24 (2015): 5187-5193; Rhode P. R., et al. “Comparison of the superagonist complex, ALT-803, to IL15 as cancer immunotherapeutics in animal models.” Cancer Immunology Research 4.1 (2016): 49-60; Mishra, A., et al. “Molecular pathways: interleukin-15 signaling in health and in cancer.” Clinical Cancer Research 20.8 (2014): 2044-2050; and Quéméner, A., et al. “IL-15Rα membrane anchorage in either cis or trans is required for stabilization of IL-15 and optimal signaling.” Journal of Cell Science 133.5 (2020): jcs236802; each of which is incorporated by reference in its entirety.

In one aspect, in order to fulfill the aforementioned first aim, the present disclosure provides engineered NK cells capable of autonomous growth and having prolonged persistence in vitro, enabling features that confer the engineered NK cells to serve as an ideal off-the-shelf and/or allogeneic NK cell drug platform.

As used herein, the term “autonomous growth” is referred to as when a population of cells are cultured in vitro, the total cell number thereof can remain increased or remain substantially unchanged after at least 7 days (e.g., at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, or at least 35 days) as compared to initial cell number on Day 0), of culture without any supporting cytokines. The term “prolonged persistence” can be regarded to have a similar definition as “autonomous growth.” Exemplary supporting cytokines for NK cells include, e.g., IL-2, IL-6, IL-7, IL-12, IL-15, IL-18, IL-21, FMS-like Tyrosine Kinase 3 Ligand (Flt-3L), Human Stem Cell Factor (SCF), Thrombopoietin (TPO), and type I interferons. In some embodiments, the supporting cytokines described herein do not include those contained in a serum replacement (e.g., Gibco, Cat #: A2506101). Details of supporting cytokines can be found, e.g., in Zwirner, N. W., et al. “Cytokine regulation of natural killer cell effector functions.” Biofactors 36.4 (2010): 274-288; and Wu, S. Y., et al. “Natural killer cells in cancer biology and therapy.” Molecular Cancer 19 (2020): 1-26, each of which is incorporated herein by reference in its entirety.

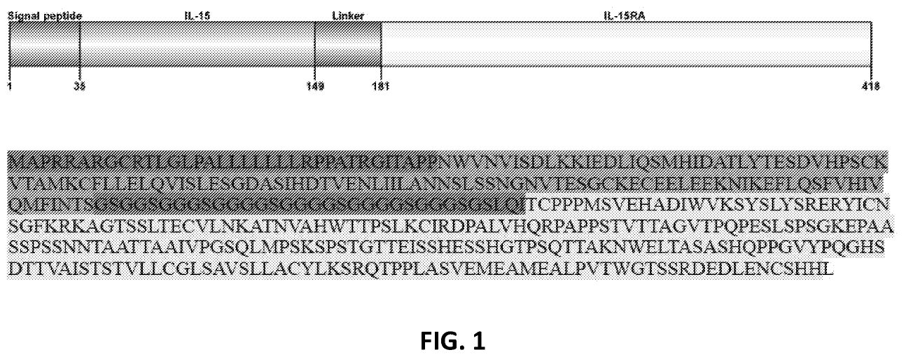

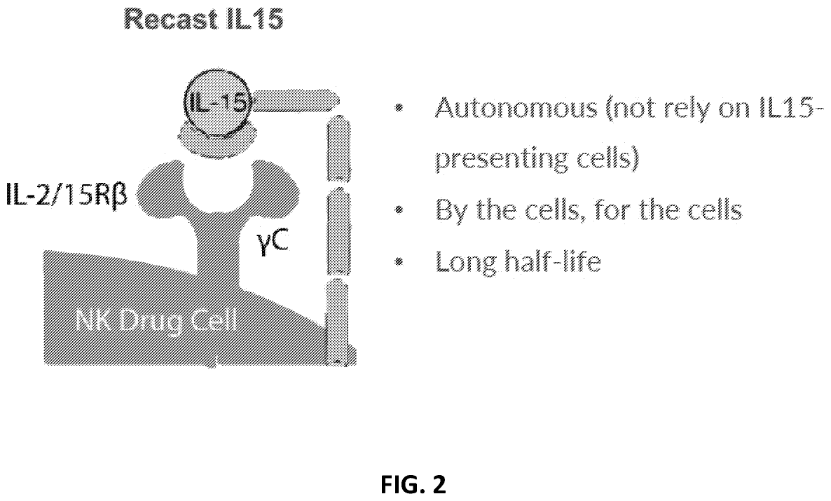

Specifically, the NK cells isolated from a particular source (e.g., cord blood) are engineered to express a transgene encoding an IL15 receptor alpha subunit (IL15RA) connected IL15 (i.e., “Recast-IL15”). Recast-IL15 is substantially a chimeric protein of interleukin 15 (i.e., IL-15 or IL15) and the alpha subunit of its receptor (i.e., IL15Rα), which are connected by an engineered linker region. The domains and sequences of the transgene construct are shown in FIG. 1 and Table 1 below. The construct can also include a signal peptide, which is connected to the N-terminus of the Recast-IL15 and is configured to ensure the proper localization of the Recast-IL15 on the cell surface of NK cells due to the transmembrane domain of IL15Rα. Upon intracellular expression in the NK cells, the signal peptide can be cleaved. As a result, Recast-IL15 can properly express on the cell surface of the NK cells and remains membrane-bound. The working mechanism for the Recast-IL15 to enable the NK cells for autonomous growth and to increase the NK cell persistency in vivo is shown in FIG. 2.

TABLE 1
Domain and sequence information of Recast-IL15.

| Domain | Amino Acid Sequence (SEQ ID NO) |
|---|---|
| Signal peptide | MAPRRARGCRTLGLPALLLLLLLRPPATRGITAPP (SEQ ID NO: 1) |
| IL-15 domain | NWVNVISDLKKIEDLIQSMHIDATLYTESDVHPSCKVTAMKCFLLELQVISLESGD |
| | ASIHDTVENLIILANNSLSSNGNVTESGCKECEELEEKNIKEFLQSFVHIVQMFINT |
| | S (SEQ ID NO: 2; wildtype IL15) |
| | NWVNVISDLKKIEDLIQSMHIDATLYTESDVHPSCKVTAMKCFLLELQVISLESGD |
| | ASIHDTVENLIILANDSLSSNGNVTESGCKECEELEEKNIKEFLQSFVHIVQMFINT |
| | S (SEQ ID NO: 3; mutant IL15, with the N72D mutation shown in bold |
| | and underline) |
| Linker region | GSGGSGGGSGGGGSGGGGSGGGGSGGGSGSLQI (SEQ ID NO: 4) |
| IL-15Rα (IL-15RA) | TCPPPMSVEHADIWVKSYSLYSRERYICNSGFKRKAGTSSLTECVLNKATNVAHW |
| domain | TTPSLKCIRDPALVHQRPAPPSTVTTAGVTPQPESLSPSGKEPAASSPSSNNTAAT |
| | TAAIVPGSQLMPSKSPSTGTTEISSHESSHGTPSQTTAKNWELTASASHQPPGVY |
| | PQGHSDTTVAISTSTVLLCGLSAVSLLACYLKSRQTPPLASVEMEAMEALPVTWG |
| | TSSRDEDLENCSHHL (SEQ ID NO: 5) |
| Recast-IL15 (full- | MAPRRARGCRTLGLPALLLLLLLRPPATRGITAPPNWVNVISDLKKIEDLIQSMHI |
| length) | DATLYTESDVHPSCKVTAMKCFLLELQVISLESGDASIHDTVENLIILANNSLSSNG |
| | NVTESGCKECEELEEKNIKEFLQSFVHIVQMFINTSGSGGSGGGSGGGGSGGGG |
| | SGGGGSGGGSGSLQITCPPPMSVEHADIWVKSYSLYSRERYICNSGFKRKAGTSS |
| | LTECVLNKATNVAHWTTPSLKCIRDPALVHQRPAPPSTVTTAGVTPQPESLSPSG |
| | KEPAASSPSSNNTAATTAAIVPGSQLMPSKSPSTGTTEISSHESSHGTPSQTTAKN |
| | WELTASASHQPPGVYPQGHSDTTVAISTSTVLLCGLSAVSLLACYLKSRQTPPLAS |
| | VEMEAMEALPVTWGTSSRDEDLENCSHHL (SEQ ID NO: 6; version 1, |
| | containing wildtype IL15) |
| | NWVNVISDLKKIEDLIQSMHIDATLYTESDVHPSCKVTAMKCFLLELQVISLESGD |
| | ASIHDTVENLIILANNSLSSNGNVTESGCKECEELEEKNIKEFLQSFVHIVQMFINT |
| | SGSGGSGGGSGGGGSGGGGGGGGSGGGSGSLQITCPPPMSVEHADIWVKSY |
| | SLYSRERYICNSGFKRKAGTSSLTECVLNKATNVAHWTTPSLKCIRDPALVHQRPA |
| | PPSTVTTAGVTPQPESLSPSGKEPAASSPSSNNTAATTAAIVPGSQLMPSKSPSTG |
| | TTEISSHESSHGTPSQTTAKNWELTASASHQPPGVYPQGHSDTTVAISTSTVLLC |
| | GLSAVSLLACYLKSRQTPPLASVEMEAMEALPVTWGTSSRDEDLENCSHHL |
| | (SEQ ID NO: 7; chimeric polypeptide w/o signal peptide) |

As a comparison, the working mechanism of wildtype IL15 is discussed in Waldmann, T. A. “The shared and contrasting roles of IL2 and IL15 in the life and death of normal and neoplastic lymphocytes: implications for cancer therapy.” Cancer Immunology Research 3.3 (2015): 219-227, which is incorporated herein by reference in its entirety. Wildtype IL15 can increase NK drug cells' persistency in vitro and in vivo. The NK cells typically express membrane-bound IL15Rβ and IL15Rγ subunits, and upon binding of the IL-15 engaged IL15Rα (which forms IL15-IL15Rα complex) on the IL15-presenting cells such as monocyte dendritic cells, the intracellular signaling of the IL15Rβ and IL15Rγ is activated, thereby sustaining the growth of NK cells.

As further demonstrated in the examples provided herein, such Recast-IL15-expressing NK cells can realize optimized autonomous growth for as long as approximately a month in culture without any supporting cytokines.

The Recast-IL15 design can overcome several shortfalls that are commonly associated with the use of wildtype cytokine IL15 in sustaining the growth of target NK cells in culture. Firstly, since Recast-IL15 is membrane-bound on, and constantly expressed by, the NK cells, the half-life of Recase-IL15 is no longer a concern compared with the use of wildtype cytokine IL15. Secondly, the engineered linker region allows Recast-IL15 to directly interact with IL2/15Rβ and IL15Rγ (i.e., γC) to trigger the downstream signaling pathway. Therefore, Recast-IL15's signaling mechanism is autonomous and no longer depends on an indirect signal presentation mechanism.

It should be noted that in Recast-IL15, each of the signal peptide, the IL-15 domain, the linker region, and the IL-15Rα domain may have different embodiments, as long as the engineered cytokine can be stably expressed on the cell surface of NK cells and can sustain the autonomous growth of NK cells without any supporting cytokines for at least 18 days. For example, according to some embodiments, the IL-15 domain may comprise an IL15N72D mutation (SEQ ID NO: 3, also seen in Table 1), which has been shown to cause the IL-15 variant to exhibit super agonist activity. Details can be found in WO2008143794A1, which is incorporated herein by reference in its entirety.

Because of the above mentioned advantages of Recast-IL15-expressing NK cells, it substantially provides an optimized yet general NK cell drug platform, on the basis of which a series of specific NK cell drugs directed to different purposes can be developed.

The following are to be noted. (1) In addition to NK cells, other immune cells such as lymphocytes, T cells, and tumor-infiltrating lymphocyte (TILs), etc., can also be engineered to express Recast-IL15, which may also equip these engineered immune cells with a better autonomous growth potential or with a better sustainability when in vivo or being cultured in vitro. (2) Each of the domains/regions as listed in Table 1 may be altered as long as the Recast-IL15 chimeric protein expressed in the NK cells can confer the NK cells the autonomous growth capability as defined. For example, a different signal peptide or a different linker region can be used. A different version of the IL-15 domain or of the IL-15Rα (IL-15RA) domain, such as a functional fragment or a sequence variant of the IL-15 and/or the IL-15Rα may be employed. (3) In addition to the chimeric Recast-IL15 as disclosed herein, it is possible to employ a membrane-bound IL-15 protein (i.e., the IL15 is fused with a cell-membrane-anchored molecule other than the IL-15Rα) as long as the expression of such membrane-bound IL-15 protein results in the autonomous growth of the NK cells.

In some embodiments, the cell expansion rate of NK cells expressing the Recast-IL15 (e.g., any of the Recast-IL15 described herein) is at least 10%, 20%, 30%, 40%, 50%, 60%, 70%, 80%, 90%, 1-fold, 2-fold, 3-fold, 4-fold, 5-fold, 6-fold, 7-fold, 8-fold, 9-fold, 10-fold, 15-fold, 20-fold, 50-fold, 100-fold, 200-fold, 500-fold, 1000-fold, 2000-fold, 3000-fold, 4000-fold, 5000-fold, or 10000-fold as compared to reference NK cells (e.g., NK cells not expressing the Recast-IL15).

In some embodiments, the NK cells expressing the Recast-IL15 (e.g., any of the Recast-IL15 described herein) can maintain the total cell number in a medium (e.g., without any supporting cytokines) for at least 7 days, at least 8 days, at least 9 days, at least 10 days, at least 11 days, at least 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 32 days, 33 days, 34 days, or 35 days. Herein, the term “maintain the total cell number” means the total cell number remains higher or substantially unchanged compared with the reference Day 0 of culture.

In one aspect, the disclosure is related to an engineered immune cell, expressing a fusion polypeptide comprising, optionally from N-terminus to C-terminus: a first moiety comprising all or a portion of interleukin 15 (IL15), and a second moiety comprising all or a portion of interleukin 15 receptor, alpha subunit (IL15Rα). In some embodiments, the fusion polypeptide can form a functional complex with interleukin 15 receptor, beta subunit (IL15Rβ) and interleukin 15 receptor, gamma subunit (IL15Rγ) to activate IL-15 signaling in the engineered immune cell. In some embodiments, the immune cell is a natural killer (NK) cell (e.g., a NK cell obtained from cord blood), a T cell, or a tumor-infiltrating cell (TIL).

In some embodiments, the first moiety comprises or consists of a sequence corresponding to amino acids 49-162 of human IL-15 protein (NCBI reference number: NP_000576.1). In some embodiments, the first moiety does not include the signal peptide of human IL-15 protein. In some embodiments, the first moiety comprises an amino acid sequence that is at least 80%, 85%, 90%, 95%, 96%, 97%, 98%, 99% or 100% identical to SEQ ID NO: 2 or 3. In some embodiments, the second moiety comprises or consists of a sequence corresponding to amino acids 32-267 of human IL-15Rα protein (NCBI reference number: NP_002180.1). In some embodiments, the second moiety comprises or consists of the extracellular region, the transmembrane region, and/or the cytoplasmic region of human IL-15Rα protein. In some embodiments, the second moiety does not include the signal peptide of human IL-15Rα. In some embodiments, the second moiety comprises an amino acid sequence that is at least 80%, 85%, 90%, 95%, 96%, 97%, 98%, 99% or 100% identical to SEQ ID NO: 5. In some embodiments, the first moiety and the second moiety are fused via a linker peptide (e.g., a flexible linker). In some embodiments, the linker peptide comprises an amino acid sequence that is at least 80%, 85%, 90%, 95%, 96%, 97%, 98%, 99% or 100% identical to SEQ ID NO: 4. In some embodiments, the fusion polypeptide further comprises an N-terminal signal peptide. In some embodiments, the signal peptide comprises or consists of a sequence corresponding to amino acids 1-35 of human IL-15Rα protein (NCBI reference number: NP_002180.1), optionally the signal peptide comprises an Ala residue at a position corresponding to Cys33 of human IL-15Rα protein. In some embodiments, the signal peptide comprises the signal peptide of human IL-15Rα protein. In some embodiments, the signal peptide comprises an amino acid sequence that is at least 80%, 85%, 90%, 95%, 96%, 97%, 98%, 99% or 100% identical to SEQ ID NO: 1. In some embodiments, the fusion polypeptide comprises an amino acid sequence that is at least 80%, 85%, 90%, 95%, 96%, 97%, 98%, 99% or 100% identical to SEQ ID NO: 6 or 7. In some embodiments, the fusion polypeptide comprises, from N-terminus to C-terminus, an IL15-Ra signal peptide, human IL-15 (without signal peptide of IL15), a flexible linker, and the extracellular region, transmembrane region, and cytoplasmic region of human IL-15Rα. In some embodiments, when a population of the immune cell is cultured in vitro, the total cell number thereof can remain increased or substantially unchanged after at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, or at least 35 days of culture in the absence of supporting cytokines (e.g., any of the supporting cytokines described herein). In some embodiments, the engineered immune cell can further express and/or secret one or more polypeptides, each comprising a target-binding domain and an Fc domain, wherein the target-binding domain can specifically bind to a target molecule on the cell surface of a target cell, and/or the Fc domain is capable of mediating antibody-dependent cellular cytotoxicity (ADCC) for the engineered immune cell. In some embodiments, the one or more polypeptides comprises an antibody or antigen-binding fragment thereof that binds to one or more antigens expressed on the surface of cancer cells (e.g., PD-L1 or any of the antigens in Table 2), and/or a peptibody that binds to one or more receptors expressed on the surface of cancer cells (e.g., IL13Rα2 or any of the receptors in Table 4).

### Ab-NK Cell Drug Platform

In a second aspect, in order to fulfill the aforementioned second aim, the disclosure further provides an engineered NK cell drug platform, termed “Ab-NK cells,” which are substantially engineered to express and secrete one or more Fc-containing antibodies and/or peptibodies that can selectively recognize and bind to corresponding cell-surface target molecules (i.e., cell-surface antigens corresponding to the Fc-containing antibodies, and/or cell-surface receptors corresponding to the Fc-containing peptibodies) that are specifically expressed on one or more target cell populations.

Upon administration of these engineered Ab-NK cells in a subject, the one or more antibodies and/or peptibodies thus secreted therefrom can specifically recognize and substantially label the one or more target cells that express the corresponding cell-surface target molecules, in turn triggering downstream cytotoxicity activities against these labelled target cells, thereby additionally materializing the cytotoxic potential for these NK cell drugs and the endogenous cells from the patient's immune system. These cytotoxicity activities may include one or any combination of the following:
(a) antibody-dependent cell-mediated cytotoxicity (ADCC) activity (e.g., for the Fc-containing antibodies) or alike (e.g., for the Fc-containing peptibodies), mediated by effector immune cells existing in the subject, such as NK cells (including exogenous and endogenous NK cells), macrophages, resident monocytes, neutrophils, and eosinophils, etc., that are recruited by these target cell surface-covering antibodies and/or peptibodies;
(b) antibody-dependent cellular phagocytosis (ADCP) activity (e.g., for the Fc-containing antibodies) or alike (e.g., for the Fc-containing peptibodies), also mediated by the above mentioned effector immune cells; and/or
(c) complement-dependent cytotoxicity (CDC) activity, mediated by the endogenous complement system in the subject.

Therefore, when used in combination, the secreted antibody/antibodies and the NK cells can substantially work in a synergic manner to mutually enhance each other's therapeutic effects.

Specifically, NK cells isolated from a particular source (e.g., cord blood) can be engineered to express one or more target transgenes that encode one or more secretable antibodies and/or one or more secretable peptibodies. Each of the antibody or peptibody can be a single-polypeptide protein or a protein complex formed by multiple polypeptides that, when secreted outside the NK cells, is capable of (1) selectively recognizing and binding to a target molecule that expresses specifically on the cell surface of a target cell, and is also capable of (2) mediating cytotoxic activity (e.g., at least one of the ADCC, ADCP, and CDC activities) against the target cell. To realize the above two functionalities, the encoded antibody or peptibody can comprise at least one target-binding domain each configured to selectively bind to a cell surface-residing target molecule corresponding thereto, and additionally comprise a fragment crystallizable (Fc) domain that is capable for mediating one or more cytotoxic activities. In some embodiments, the target-binding domain described herein is an antigen-binding domain for an antibody or antigen-binding fragment thereof described herein. In some embodiments, the target-binding domain described herein is a ligand polypeptide for a peptibody described herein.

The following descriptions are provided for the antibody theme and for the peptibody theme separately, depending on different embodiments of the disclosure.

### (1) The “Antibody” Theme

According to some embodiments, the engineered Ab-NK cells are configured to express and secrete one or more antibodies. As used herein, the term “antibody” (or “antibodies” in plural forms) is referred to as a single-polypeptide protein or a protein complex formed by multiple polypeptides that can specifically recognize a corresponding cell surface antigen of a target cell, and depending on different embodiments, can be of different forms or types, such as a single-polypeptide antibody, a multiple-polypeptide antibody (i.e., immunoglobulin (Ig)) unit (e.g., a Y-shaped antibody unit comprising two light chains and two heavy chains), or other forms.

### (A) “Single-Polypeptide” Antibody

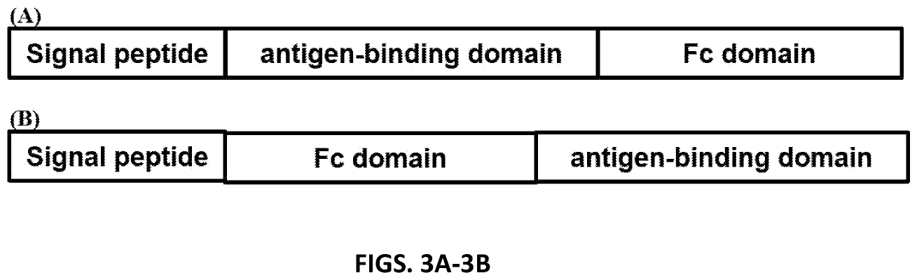

According to some embodiments, the antibody may comprise a single-polypeptide protein, comprising an antigen-binding domain and an Fc domain. In order to ensure that the translated antibody polypeptide to be properly secreted outside the NK cells, a signal peptide is additionally fused to the N-terminal of the antigen-binding domain of the antibody construct (FIG. 3A), or alternatively to the N-terminal of the Fc domain of the antibody construct (FIG. 3B). Every two neighboring domains may be connected by a flexible linker or directly connected without a linker.

In some embodiments, the antigen-binding domain is configured to specifically recognize, thereby allowing the antibody to specifically bind to, a corresponding cell-surface antigen on a target cell. There can be different embodiments for the antigen-binding domain in the antibody expressed in the NK cells. According to some embodiments, the antigen-binding domain may comprise a single-chain variable fragment (scFv) domain that can specifically bind to a corresponding cell-surface antigen on the target cell. Herein, the scFv domain is interpreted as a fusion polypeptide fused between two variable domains, from the light chain and from the heavy chain of a regular antibody respectively. According to some other embodiments, the antigen-binding domain may comprise a single monomeric variable antibody domain. Other embodiments may also be possible for the antigen-binding domain, and there is no limitation herein.

In some embodiments, the Fc domain is configured to be cytotoxicity-capable, allowing the antibody polypeptide that is expressed and secreted from the Ab-NK cells to be able to mediate at least one of antibody-dependent cell-mediated cytotoxicity (ADCC) activity, antibody-dependent cellular phagocytosis (ADCP) activity, and/or complement-dependent cytotoxicity (CDC) activity, against the target cell. There can be different embodiments for the Fc domain in each such polypeptide.

Optionally, the Fc domain may comprise a sequence of IgG, IgA, IgD, IgM and IgE. The Fc domain may further comprise one or more (e.g., 1, 2, 3, 4, 5, 6, 7, 8, 9, or 10) of the following mutations of IgG1 Fc region: Ser239Asp, Ala330Leu, Ile332Glu, Phe243Leu, Arg292Pro, Tyr300Leu, Val305Ile, Pro396Leu, Met428Leu, and Asn434Ser according to EU numbering. An antibody with an Fc domain containing certain combinations of these mutations have been shown to result in enhanced ADCC activity, enhanced ADCP activity, enhanced CDC activity, and/or prolonged half-life of the antibody polypeptide (see e.g., U.S. Pat. No. 7,317,091B2, U.S. Pat. No. 8,039,592B2, US20040132101A1, U.S. Pat. No. 7,786,270B2, WO2009086320A1, WO2006053301A2, U.S. Pat. No. 8,088,376B2, and U.S. Pat. No. 8,394,925B2, whose disclosures are hereby incorporated by reference in their entireties). Details of the EU numbering system can be found, e.g., in Lobner, E., et al. “Engineered IgG1-Fc-one fragment to bind them all.” Immunological Reviews 270.1 (2016): 113-131, and Kabat, E. A., et al. “Sequences of Proteins of Immunological Interest, 5th edit.” National Institutes of Health, Bethesda, MD (1991); each of which is incorporated herein by reference in its entirety.

According to some embodiments, the IgG1 Fc domain may comprise a combination of Ser239Asp and Ile332Glu, and/or Ala330Leu according to EU numbering to confer an enhanced ADCC and ADCP to the antibody polypeptide containing such modified Fc domain.

According to some embodiments, the IgG1 Fc domain may comprise a combination of Phe243Leu, Arg292Pro, Tyr300Leu, Val305Ile and/or Pro396Leu according to EU numbering to confer an enhanced ADCC to the antibody polypeptide containing such modified Fc domain.

According to some embodiments, the IgG1 Fc domain may comprise a combination of Met428Leu and/or Asn434Ser according to EU numbering to confer a prolonged half-life to the antibody polypeptide containing such modified Fc domain.

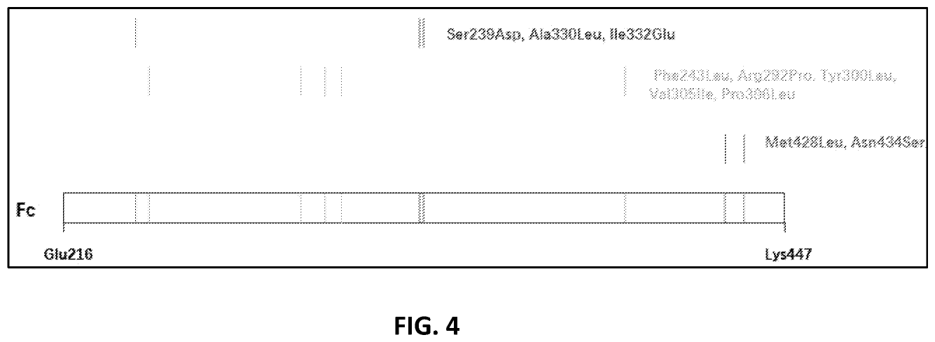

According to some preferred embodiments, the Fc domain for an antibody polypeptide carried by the Ab-NK cells may comprise the combination of all of these above 10 mutations (FIG. 4)

It is noted that there can be other mutations/modifications of the Fc domain that allow the antibody or antibody-like polypeptide containing such engineered Fc domain to have optimized ADCC activity, ADCP activity, complement-dependent cytotoxicity (CDC) activity, and/or half-life. These mutations may include Cys221Asp, Asp222Cys, Leu234Tyr, Gly236Ala, Gly236Trp, Ser267Glu, His268Phe, Ser298Ala, Ser324Thr, Lys325Trp, Lys326Ala, Lys326Met, Glu333Ala, Glu333Ser, Lys334Ala, and/or Glu345Arg according to EU numbering.

Because of the presence of the functional Fc domain in each of the one or more antibody polypeptides, when the one or more antibodies are secreted out of the Ab-NK cells, they can mediate the antibody-dependent cytotoxicity (ADCC) of the NK cells against target cells that express the corresponding antigens. Meanwhile, the antibody triggers multiple layers of immune responses, such as ADCC, ADCP and CDC. Furthermore, the antibody may provide pharmaceutical functions other than target recognition, for example, neutralizing, blocking and/or agonizing certain signaling pathways.

### (B) “Multiple-Polypeptide” Antibody

According to some embodiments, the antibody may comprise a multiple-polypeptide protein complex. Such protein complex may optionally be in a form of a traditional immunoglobulin (Ig) comprising two light chains and two heavy chains connected by disulfide bonds and having a Y-shape. Such multiple-polypeptide antibody substantially includes two antigen-binding domains, each comprising the three complementarity-determining regions (CDRs) from each of the light chain and heavy chain. Such multiple-polypeptide antibody further includes one or more Fc domains, each comprising the constant domain from one heavy chain. The multiple-polypeptide antibody may optionally be in a different form, such as comprising only one light chain and one heavy chain, comprising a partial version of an immunoglobulin (Ig), or others.

In any embodiments of the multiple-polypeptide antibody as described herein, the Fc domain contained therein can be engineered to comprise one or more mutations as described above to have an improved ADCC/ADCP/CDC activities and/or half-life.

Optionally, the single-polypeptide antibody or the multiple-peptide antibody that is expressed and secreted by the Ab-NK cells may comprise an antigen-binding domain as disclosed in one of the references listed in Table 2, or a modification thereof. The contents of these cited references are incorporated in this present disclosure in their entirety.

TABLE 2
List of antigens that are targetable by currently developed antibodies.

| Name | Full-name, alias, or other note | References |
|---|---|---|
| BCMA | B cell maturation antigen | U.S. Pat. No. 9,273,141B2, |
| | | WO2014068079A1, |
| | | WO2014122144A1 |
| Caudin18.2 | | US20150252103A1, |
| | | U.S. Pat. No. 10,053,512B2, |
| | | WO2016165762A1 |
| CCR4 | C-C Motif Chemokine Receptor 4 | U.S. Pat. No. 10,023,649B2, |
| | | U.S. Pat. No. 9,675,625B2 |
| CD10 | | JP2007129903A, |
| | | CN103880960A |
| CD123 | the α chain of the interleukin | WO2016201065A1, |
| | 3 receptor | U.S. Pat. No. 9,969,807B2 |
| CD147 | basigin | US20170037129A1, |
| | | US20170101469A1 |
| CD171 | neural cell adhesion molecule L1 | U.S. Pat. No. 10,400,037B2, |
| | | U.S. Pat. No. 9,777,060B2, |
| | | U.S. Pat. No. 8,138,313B2 |
| CD19 | | U.S. Pat. No. 7,902,338B2, |
| | | U.S. Pat. No. 8,097,703B2, |
| | | U.S. Pat. No. 9,260,530B2, |
| | | U.S. Pat. No. 7,109,304B2 |
| CD20 | | U.S. Pat. No. 8,101,179B2, |
| | | U.S. Pat. No. 7,682,612B1 |
| CD22 | | U.S. Pat. No. 9,139,649B2, |
| | | U.S. Pat. No. 8,591,889B2, |
| | | CN103214578B, |
| | | US20160229911A1 |
| CD276 | B7 Homolog 3 (B7-H3) | CA2680111A1, |
| | | EP3193933A1, |
| | | U.S. Pat. No. 10,604,583B2 |
| CD319 | SLAMF7 | EP2068874B1, |
| | | U.S. Pat. No. 8,603,477B2 |
| CD33 | | U.S. Pat. No. 11,136,390B2, |
| | | U.S. Pat. No. 10,787,514B2, |
| | | U.S. Pat. No. 8,759,494B2 |
| CD38 | cyclic ADP ribose hydrolase | U.S. Pat. No. 8,153,765B2, |
| | | U.S. Pat. No. 8,926,969B2, |
| | | U.S. Pat. No. 10,766,965B2 |
| CD70 | a ligand of CD27 | U.S. Pat. No. 8,663,642B2, |
| | | U.S. Pat. No. 7,491,390B2, |
| | | US20190338039A1 |
| CLL-1 | C-type lectin-like molecule-1 | US20130295118A1, |
| | | U.S. Pat. No. 10,501,545B2, |
| | | U.S. Pat. No. 8,536,310B2, |
| | | U.S. Pat. No. 11,084,877B2 |
| DLL3 | Delta-like ligand 3 | US20120328624A1, |
| | | U.S. Pat. No. 9,107,961B2, |
| | | US20210380715A1 |
| EGFR | epidermal growth factor receptor | U.S. Pat. No. 10,759,860B2, |
| | | U.S. Pat. No. 7,598,350B2, |
| EGFRvIII | an active mutant of EGFR | U.S. Pat. No. 9,676,858B2, |
| | | US20170327585A1 |
| EpCAM | Epithelial Cell Adhesion Molecule | U.S. Pat. No. 8,637,017B2; |
| | | CN101970497B |
| FLT3 | FLT3 encodes a class III receptor | U.S. Pat. No. 8,071,099B2; |
| | tyrosine kinase, is an important | CN102770453B |
| | cytokine receptor involved in | |
| | normal hematopoiesis | |
| FRα | a membrane protein, which binds | CN107011439B; |
| | folicacid | U.S. Pat. No. 9,624,297B2 |
| GD2 | GD2 is a disialoganglioside expressed | U.S. Pat. No. 10,287,365B2; |
| | on tumors of neuroectodermal origin | AU2014228772B2 |
| GPC3 | Glypican 3 | JP6579640B2; |
| | | U.S. Pat. No. 9,926,377B2 |
| HER2 | human epidermal growth factor | CN107074962B; |
| | receptor 2 | U.S. Pat. No. 9,518,118B2 |
| HGFR | hepatocyte growth factor receptor | EP1641828B1; |
| | | U.S. Pat. No. 6,207,152B1 |
| IL13Ralpha2 | IL13 receptor alpha2 | KR101763499B1; |
| | | WO2008146911A9 |
| Mesothelin | | U.S. Pat. No. 10,022,452B2; |
| | | JP5788443B2 |
| MG7 | a glycosylated protein sequence from | CN110684107B |
| | carcinoembryonic antigen (CEA) | |
| MUC1 | Mucin 1 | U.S. Pat. No. 8,722,856B2; |
| | | JP6895890B2 |
| MUC16 | Mucin 16 | U.S. Pat. No. 11,066,480B2 |
| Nectin4 | Nectin Cell Adhesion Molecule 4 | U.S. Pat. No. 11,292,837B2; |
| | | U.S. Pat. No. 11,274,160B2 |
| PSCA | Prostate Stem Cell Antigen | U.S. Pat. No. 6,790,939B2; |
| | | U.S. Pat. No. 8,013,128B2 |
| ROR1 | Receptor Tyrosine Kinase Like | U.S. Pat. No. 11,155,615B2; |
| | Orphan Receptor 1 | CN103429618B |
| ROR2 | Receptor Tyrosine Kinase Like | US20180127503A1; |
| | Orphan Receptor 2 | US20210347892A1 |
| TACI | Transmembrane activator and CAML | US20050043516A1 |
| | interactor (TACI) is transmembrane | |
| | protein of the TNF receptor | |
| | superfamily found predominantly | |
| | on the surface of B cells | |
| TRBC1 | T Cell Receptor Beta Constant 1 | US20200140549A1 |
| TSLPR | thymic stromal lymphopoietin | U.S. Pat. No. 8,344,110B2 |
| VEGFR | vascular endothelial growth | U.S. Pat. No. 10,138,300B2; |
| | factor receptor | U.S. Pat. No. 8,128,932B2 |

Optionally, the single-polypeptide antibody or the multiple-peptide antibody that is expressed and secreted by the Ab-NK cells may be configured to be in a prodrug form, which remains inactive until certain condition is met when the inactive prodrug is activated to become the functional drug form.

In one scenario, an antibody in its prodrug form may remain inactive until reaching target tissues when the antibody is converted from the inactive prodrug form to an active drug form. As such, the unwanted off-target effects of the Ab-NK cells towards the cells in normal tissues, which may also express a low level of the target antigens, can be ameliorated, reduced, or even totally avoided. Examples of such target antigens can include EGFR and Her2.

According to some embodiments, the antibody expressed and secreted from the Ab-NK cells further comprises a masking peptide, and the masking peptide and the rest of the polypeptide are fused with each other via a flexible linker that is degradable by a proteolytic enzyme that is specifically or enrichingly expressed in the diseased tissue where the target cells are located (e.g., the diseased tissue is tumor, and the proteolytic enzyme is substantially a tumor-associated enzyme). In one example, the flexible linker may contain one or more matrix metalloproteinase 9 (MMP9) degradable/cleavage sites, i.e., VPLSLYS (SEQ ID NO: 26) or SPLGLA (SEQ ID NO: 27). Other proteolytic enzyme may include MMP1, MMP2, MMP3, MMP7, MMP14, and their specific cleavage sites are disclosed in WO2002038796A2, which is incorporated herein by reference in its entirety. Each of the cleavage sites may be used as the disease-specific cleavage site for the flexible linker. Alternatively, the flexible linker may contain a cleavage site that is specific for an exogenously supplied enzyme. In some embodiments, the masking peptide described herein can block the Fc domain of an antibody or antigen-binding fragment thereof (e.g., any of the antibodies or antigen-binding fragments thereof described herein) or a peptibody (e.g., any of the peptibodies described herein). In some embodiments, the masking peptide can block the interaction between the Fc domain and an Fc receptor (e.g., CD16), thereby reducing or silencing one or more Fc effector activities (e.g., ADCC). In some embodiments, the masking peptide described herein can block the target-binding domain (e.g., an antigen-binding domain) of an antibody or antigen-binding fragment thereof (e.g., any of the antibodies or antigen-binding fragments thereof described herein) or a peptibody (e.g., any of the peptibodies described herein). In some embodiments, the masking peptide can block the target-binding domain (e.g., an antigen-binding domain) binding to its target molecule (e.g., an antigen).

Specifically, after secretion from the Ab-NK cells, the prodrug-form antibody remains inactive in circulation or in tissues other than the diseased tissues due to (1) the occlusion of the antigen recognition sites (e.g., the complement determination regions (CDRs)) in the antigen-binding domain, and/or to (2) the occlusion of the Fc domain, of the antibody polypeptide by the masking peptide. However, when the antibody is in the target diseased tissue, the masking peptide is removed through the degradation of the flexible linker by the corresponding proteolytic enzyme, causing the previously blocked antigen-binding domain or the previously blocked Fc domain to be exposed and accessible, thereby activating the antibody from the original prodrug form to the activated drug form. Depending on different manners, thus activated drug-form antibody may:
(1) selectively recognize and bind to the corresponding target antigens that are specifically expressed on the cell surface of the target cells because the antigen-binding domain of the antibody polypeptide is exposed by removing the original occlusion by the masking peptide, or
(2) allow the now exposed Fc domain of the antibodies that have accumulated on the cell-surface of the target cells to be able to recruit effector immune cells (e.g., exogenous Ab-NK cells, or endogenous NK cells macrophages, resident monocytes, neutrophils, and eosinophils, etc., by binding to the Fc receptors (FcγRs) expressed thereon) or activate the complement system, together leading to the activation of the ADCC/ADCP/CDC activities against the target cells.

Herein, according to a first embodiment, the masking peptide is configured to block the antigen recognition sites in the antigen-binding domain of the antibody polypeptide, and as such, the masking peptide can be arranged between the signal peptide and the antigen-binding domain or the C-terminal of the Fc domain, and the degradable flexible linker can connect the masking peptide and the antigen-binding domain. Such a design has been successfully applied in the EGFR and Her2 monoclonal antibodies as described in U.S. Pat. No. 8,895,702B2 and U.S. Pat. No. 11,186,642B2, whose contents, including the sequence information for the masking peptide and the flexible and MMP9-degradable linker, are hereby incorporated by reference in their entirety.

According to a second embodiment, the masking peptide is configured to block the Fc domain of the antibody polypeptide, and as such, the masking peptide can be arranged to the C-terminal of the Fc domain or the N-terminal of the antigen-binding domain, and the degradable flexible linker is arranged to connect the Fc domain and the masking peptide. Such a design is described in Elter, A., et al. “Protease-activation of fc-masked therapeutic antibodies to alleviate off-tumor cytotoxicity.” Frontiers in Immunology 12 (2021): 715719, whose contents, including the sequence information for the masking peptide and the flexible and MMP9-degradable linker, are hereby incorporated by reference in their entirety.

In another scenario, an antibody in its prodrug form may remain inactive until being activated by a controlled supply of certain activating agent such as an activating enzyme that specifically degrades a degradable linker between a masking moiety and the antibody (just like the above examples). Other scenarios may be possible as well.

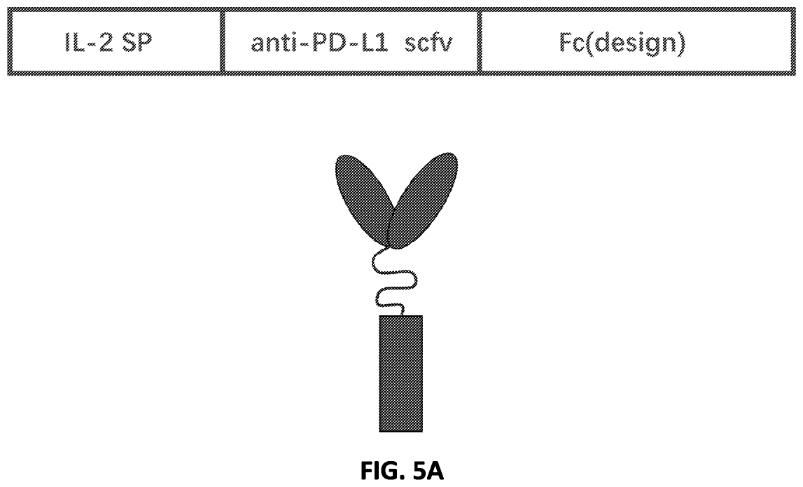

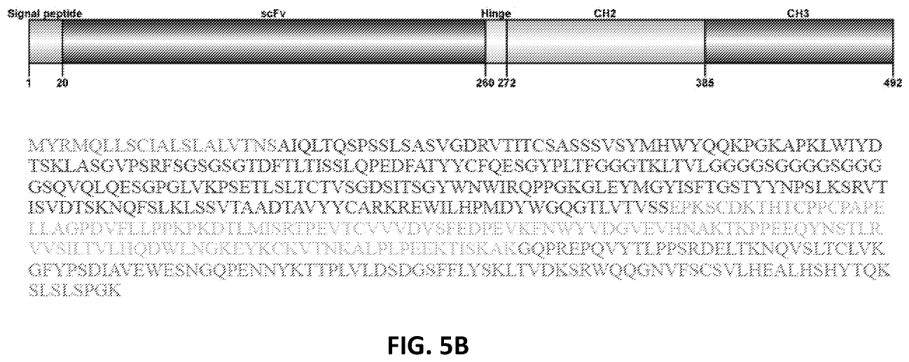

In particular, it is noted that according to some embodiments, the Ab-NK cells are engineered to express and secrete an anti-PD-L1 antibody (also called PD-L1 antibody), which is substantially a “single-polypeptide” antibody (see FIGS. 5A-5B) and such Ab-NK cells can be termed as “PD-L1-guide and cytolysis-linked NK (short as “Pluck-NK” hereinafter)” cells, which are designed as an off-the-shelf and universal cell drug that can be applied for a broad spectrum of cancer types, including, e.g., lung cancer, bladder cancer, TN breast cancer, liver cancer and liver metastasis, etc.

Herein, the anti-PD-L1 antibody may, according to some embodiments, have a structure and a sequence as illustrated in FIGS. 5A-5B and Table 3 below.

TABLE 3
Domain and sequence information of the anti-PD-L1 antibody.

| Domain | Amino Acid Sequence (SEQ ID NO) |
|---|---|
| signal peptide | MYRMQLLSCIALSLALVTNS (SEQ ID NO: 8) |
| scFv domain | Version 1: Humanized (L8 clone) |
| | VL domain |
| | AIQLTQSPSSLSASVGDRVTITCSASSSVSYMHWYQQKPGKAPKLWIYDTSKLASGVP |
| | SRFSGSGSGTDFTLTISSLQPEDFATYYCFQESGYPLTFGGGTKLTVL (SEQ ID NO: 9) |
| | VH domain |
| | QVQLQESGPGLVKPSETLSLTCTVSGDSITSGYWNWIRQPPGKGLEYMGYISFTGSTY |
| | YNPSLKSRVTISVDTSKNQFSLKLSSVTAADTAVYYCARKREWILHPMDYWGQGTLV |
| | TVSS (SEQ ID NO: 10) |
| | flexible linker |
| | GGGGSGGGGSGGGGS (SEQ ID NO: 11) |
| | full scFv domain |
| | AIQLTQSPSSLSASVGDRVTITCSASSSVSYMHWYQQKPGKAPKLWIYDTSKLASGVP |
| | SRFSGSGSGTDFTLTISSLQPEDFATYYCFQESGYPLTFGGGTKLTVLGGGGSGGGGS |
| | GGGGSQVQLQESGPGLVKPSETLSLTCTVSGDSITSGYWNWIRQPPGKGLEYMGYIS |
| | FTGSTYYNPSLKSRVTISVDTSKNQFSLKLSSVTAADTAVYYCARKREWILHPMDYWG |
| | QGTLVTVSS (SEQ ID NO: 12) |
| | Version 2: Murine (L2 clone) |
| | VL domain |
| | ENVLTQSPAIMSASPGEKVTMTCSASSSVSYMHWYQQKSLTSPKLWIYDTSKLASG |
| | VPGRFSGSGSGNFYSLTISSMEAEDVATYYCFQESGYPLTFGAGTKLELK (SEQ ID |
| | NO: 13) |
| | VH domain |
| | EVQLQESGPSLVKPSQTLSLTCSVTGDSITSGYWNWIRKFPENKLEYMGYISFTGSTY |
| | YNPSLKSRISITRDTSKNQYYLQLNSVTTEDTATYYCARKREWILHPMDYWGQGTSV |
| | TVSS (SEQ ID NO: 14) |
| | full scFv domain |
| | ENVLTQSPAIMSASPGEKVTMTCSASSSVSYMHWYQQKSLTSPKLWIYDTSKLASGV |
| | PGRFSGSGSGNFYSLTISSMEAEDVATYYCFQESGYPLTFGAGTKLELKGGGGSGGGG |
| | SGGGGSEVQLQESGPSLVKPSQTLSLTCSVTGDSITSGYWNWIRKFPENKLEYMGYIS |
| | FTGSTYYNPSLKSRISITRDTSKNQYYLQLNSVTTEDTATYYCARKREWILHPMDYWG |
| | QGTSVTVSS (SEQ ID NO: 15) |
| Hinge region | EPKSCDKTHTCP (SEQ ID NO: 16) |
| Fc-CH2 domain | PCPAPELLAGPDVFLLPPKPKDTLMISRTPEVTCVVVDVSFEDPEVKFNWYVDGVEV |
| | HNAKTKPPEEQYNSTLRVVSILTVLHQDWLNGKEYKCKVTNKALPLPEEKTISKAK |
| | (SEQ ID NO: 17) |
| Fc-CH3 domain | GQPREPQVYTLPPSRDELTKNQVSLTCLVKGFYPSDIAVEWESNGQPENNYKTTPLVL |
| | DSDGSFFLYSKLTVDKSRWQQGNVFSCSVLHEALHSHYTQKSLSLSPGK (SEQ ID |
| | NO: 18) |
| anti-PD-L1 | MYRMQLLSCIALSLALVTNSAIQLTQSPSSLSASVGDRVTITCSASSSVSYMHWYQQK |
| (full-length) | PGKAPKLWIYDTSKLASGVPSRFSGSGSGTDFTLTISSLQPEDFATYYCFQESGYPLTFG |
| | GGTKLTVLGGGGSGGGGSGGGGSQVQLQESGPGLVKPSETLSLTCTVSGDSITSGY |
| | WNWIRQPPGKGLEYMGYISFTGSTYYNPSLKSRVTISVDTSKNQFSLKLSSVTAADTA |
| | VYYCARKREWILHPMDYWGQGTLVTVSSEPKSCDKTHTCPPCPAPELLAGPDVFLLP |
| | PKPKDTLMISRTPEVTCVVVDVSFEDPEVKFNWYVDGVEVHNAKTKPPEEQYNSTL |
| | RVVSILTVLHQDWLNGKEYKCKVTNKALPLPEEKTISKAKGQPREPQVYTLPPSRDEL |
| | TKNQVSLTCLVKGFYPSDIAVEWESNGQPENNYKTTPLVLDSDGSFFLYSKLTVDKSR |
| | WQQGNVFSCSVLHEALHSHYTQKSLSLSPGK (SEQ ID NO: 19) |
| | MYRMQLLSCIALSLALVTNSENVLTQSPAIMSASPGEKVTMTCSASSSVSYMHWYQ |
| | QKSLTSPKLWIYDTSKLASGVPGRFSGSGSGNFYSLTISSMEAEDVATYYCFQESGYPL |
| | TFGAGTKLELKGGGGSGGGGSGGGGSEVQLQESGPSLVKPSQTLSLTCSVTGDSITS |
| | GYWNWIRKFPENKLEYMGYISFTGSTYYNPSLKSRISITRDTSKNQYYLQLNSVTTEDT |
| | ATYYCARKREWILHPMDYWGQGTSVTVSSEPKSCDKTHTCPPCPAPELLAGPDVFLL |
| | PPKPKDTLMISRTPEVTCVVVDVSFEDPEVKFNWYVDGVEVHNAKTKPPEEQYNST |
| | LRVVSILTVLHQDWLNGKEYKCKVTNKALPLPEEKTISKAKGQPREPQVYTLPPSRDEL |
| | TKNQVSLTCLVKGFYPSDIAVEWESNGQPENNYKTTPLVLDSDGSFFLYSKLTVDKSR |
| | WQQGNVFSCSVLHEALHSHYTQKSLSLSPGK (SEQ ID NO: 20) |

The mechanism of action (MOA) for the Pluck-NK cell drug is detailed as follows. Blockade of the PD-1/PD-L1 immune checkpoint has shown apparent and durable clinical efficacy. The PD-1/PD-L1 immune checkpoint can be blocked by antibodies against either PD-1 or PD-L1. Pluck-NK cells are equipped with a self-secreting full-length anti-PD-L1 antibody or an antigen-binding fragment thereof (e.g., scFv). PD-L1 was selected as the target, instead of PD-1, because PD-L1 is located on the tumor cell surface, therefore, is a tumor target. To be used in combination with NK cells, the anti-PD-L1 antibody can not only serve as an immune checkpoint inhibitor, but also guide NK cells to the target cancer cell via ADCC. This design can make better use of both the NK cells and the anti-PD-L1 antibody. In ADCC, the Fc receptor (CD16) on NK cell surface recognizes the Fc region of the antibody. Therefore, Pluck-NK cells are equipped with a full-length anti-PD-L1 antibody or an antigen-binding fragment thereof (e.g., scFv). The Fc region of the antibody can be configured to carry the three clusters of engineered amino acid alterations as described above and illustrated in FIG. 4. These features are designed (1) to elongate the antibody's half-life; (2) to enhance ADCC; (3) to enhance antibody-dependent cellular phagocytosis (ADCP, a macrophage-mediated immune response); (4) to enhance complement-dependent cytotoxicity (CDC, an immune response mediated by the complement cascade). These designs are aimed to maximize the therapeutic capacity of both the NK cells and the anti-PD-L1 antibody by triggering multiple layers of immune responses to target the tumor cells.

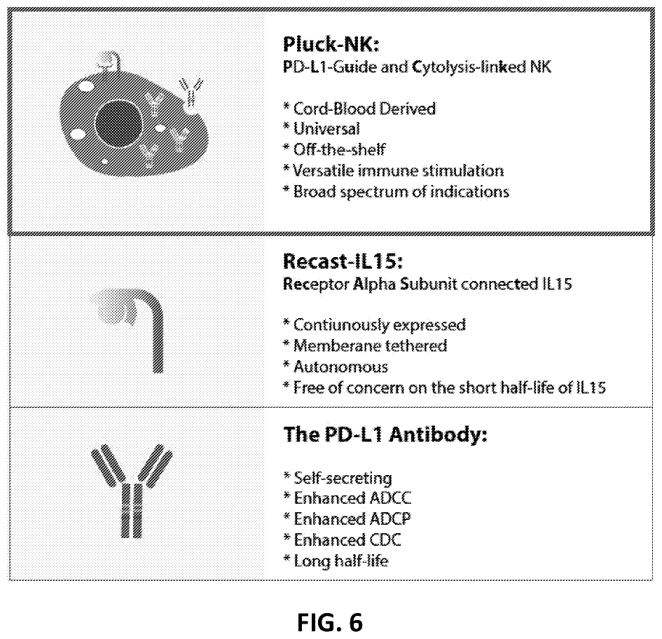

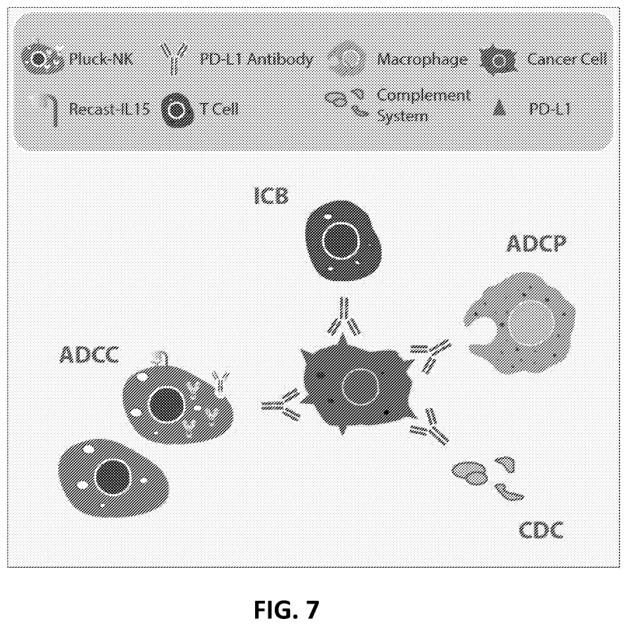

Herein, it is further noted that according to some embodiments, the Pluck-NK cells can be preferably further combined with the aforementioned autonomously growing NK cell drug platform, i.e., NK cells engineered to simultaneously express “Recast IL15” to thereby be able to realize an optimized autonomous growth, and to express the “anti-PD-L1” antibody to thereby obtain the above mentioned multiple functionalities of immune checkpoint block (ICB), and cytotoxicity related to the ADCC/ADCP/CDC. Such engineered NK cells are termed as “Pluck-NK” cells, and the key features and the mechanism of action (MOA) of the Pluck-NK cells are summarized and illustrated in FIG. 6 and FIG. 7, respectively.

In one aspect, the disclosure is related to an engineered immune cell, expressing and/or secreting a fusion polypeptide comprising, optionally from N-terminus to C-terminus: a first moiety comprising a single-chain variable fragment (scFv) that binds to an antigen expressed on the surface of cancer cells (e.g., PD-L1 or any of the antigens in Table 2), and a second moiety comprising an fragment crystallizable (Fc) region. In some embodiments, the immune cell expresses an Fc receptor (e.g., CD16) on the cell surface that can recognize the Fc region of the fusion polypeptide and mediate antibody-dependent cellular cytotoxicity (ADCC) for the engineered immune cell. In some embodiments, the immune cell is a natural killer (NK) cell (e.g., a NK cell obtained from cord blood), a T cell, or a tumor-infiltrating cell (TIL). In some embodiments, the scFv comprises, optionally from N-terminus to C-terminus, a light chain variable region (VL) having an amino acid sequence that is at least 80%, 85%, 90%, 95%, 96%, 97%, 98%, 99% or 100% identical to SEQ ID NO: 9 or 13, and a heavy chain variable region (VH) having an amino acid sequence that is at least 80%, 85%, 90%, 95%, 96%, 97%, 98%, 99% or 100% identical to SEQ ID NO: 10 or 14. In some embodiments, the VL and the VH are connected via a linker peptide (e.g., a flexible linker). In some embodiments, the linker peptide comprises an amino acid sequence that is at least 80%, 85%, 90%, 95%, 96%, 97%, 98%, 99% or 100% identical to SEQ ID NO: 11. In some embodiments, the scFv comprises an amino acid sequence that is at least 80%, 85%, 90%, 95%, 96%, 97%, 98%, 99% or 100% identical to SEQ ID NO: 12 or 15. In some embodiments, the Fc region comprises a hinge region, a CH2 domain and a CH3 domain of human IgG (e.g., IgG1). In some embodiments, the hinge region comprises an amino acid sequence that is at least 80%, 85%, 90%, 95%, 96%, 97%, 98%, 99% or 100% identical to SEQ ID NO: 16, the CH2 domain comprises an amino acid sequence that is at least 80%, 85%, 90%, 95%, 96%, 97%, 98%, 99% or 100% identical to SEQ ID NO: 17, and/or the CH3 domain comprises an amino acid sequence that is at least 80%, 85%, 90%, 95%, 96%, 97%, 98%, 99% or 100% identical to SEQ ID NO: 18. In some embodiments, the Fc region includes any of the substitutions or mutations described herein (e.g., any of the mutations in FIG. 4). In some embodiments, the fusion polypeptide further comprises an N-terminal signal peptide. In some embodiments, the signal peptide comprises or consists of a sequence corresponding to amino acids 1-20 of human IL-2 protein (NCBI reference number: NP_000577.2). In some embodiments, the signal peptide is the signal peptide of human IL-2. In some embodiments, the signal peptide comprises an amino acid sequence that is at least 80%, 85%, 90%, 95%, 96%, 97%, 98%, 99% or 100% identical to SEQ ID NO: 8. In some embodiments, the fusion polypeptide comprises an amino acid sequence that is at least 80%, 85%, 90%, 95%, 96%, 97%, 98%, 99% or 100% identical to SEQ ID NO: 19 or 20. In some embodiments, the engineered immune cell described herein also expresses another fusion polypeptide comprising, optionally from N-terminus to C-terminus: a first moiety comprising all or a portion of interleukin 15 (IL15), and a second moiety comprising all or a portion of interleukin 15 receptor, alpha subunit (IL15Rα), in some embodiments, the fusion polypeptide can form a functional complex with IL15Rβ and IL15Rγ to activate IL-15 signaling in the engineered immune cell.

### (2) The “Peptibody” Theme:

In addition to expressing and secreting one or more antibodies as described above in Theme (A), according to some embodiments, the engineered Ab-NK cells are configured to alternatively or combinatorially express and secrete one or more peptibodies. As used herein, the term “peptibody” (or “peptibodies” in plural forms) is referred to as a polypeptide or protein complex, that comprises a biologically active polypeptide domain fused with an Fc domain: the biologically active polypeptide domain (i.e., target-binding domain) is configured to selectively recognize and bind to a target molecule on the cell surface of a target cell, and the Fc domain is similarly configured to be cytotoxicity-capable.

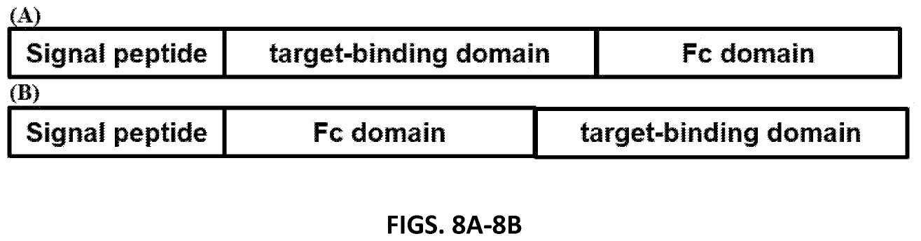

The target-binding domain and the Fc domain of the peptibody substantially allows the peptibody that is expressed and secreted from the Ab-NK cells to be able to recognize, bind to and label the target cells, and then to recruit the effector immune cells (including the Ab-NK cells themselves) and the complement system for the ADCC-like cytotoxicity activity, ADCP-like phagocytosis activity, and/or complement-dependent cytotoxicity (CDC) activity, against the target cell. In addition, the peptibody polypeptide may be additionally fused to a signal peptide, in a manner similar to the antibody construct shown above. The construct structures for the different domains of the peptibody is illustrated in FIGS. 8A-8B.

According to some embodiments of such Ab-NK secreting peptibody, the target binding domain comprises a sequence that is derived from a ligand polypeptide which can selectively recognize and bind to one or more target molecules (e.g., target receptors) specifically or enrichingly expressing on the cell surface of the target cells. Herein, such a ligand polypeptide may optionally be a natural ligand for the target receptor, or a functional variant or fragment thereof, or alternatively may be a ligand-mimicking peptide or an artificial peptide that has been screened to display binding activities to the target receptor.

Examples of ligands, receptors, or the ligand-receptor pairs known to be implicated in certain human diseases (e.g., tumor) are listed in Table 4. Based on the ligand information directly (i.e., the ligand information is provided) or indirectly (i.e., only the receptor information is provided, but the ligand information can be obtained from the receptor information) from the table, the target-binding domain of the peptibody to be expressed and secreted by the Ab-NK cells can be designed.

TABLE 4
Examples of ligands, receptors, or ligand-receptor pairs
that are implicated in human diseases (e.g., tumor)

| | |
|---|---|
| Ligand-Receptor pairs or the receptors implicated | References |

Single receptor proteins that can be targeted by peptides
based on references and are expressed on the cell surface.

| Protein | Gene symbol | Protein | Gene symbol | |
|---|---|---|---|---|
| APN/CD13 | ANPEP | GPC3 | GPC3 | Ahmadi S et al. Nature |
| APP | APP | IL-10RA | IL10RA | Communications. |
| PD-L1 | CD274 | IL-11Rα | IL11RA | volume 13, Article |
| CD44 | CD44 | IL-13Rα2 | IL13RA2 | number: 1613 (2022) |
| P32/gC1qR | CD93 | IL-6Rα | IL6R | Pastan, I., Willingham, |
| E-cadherin | CDH1 | GP130 | IL6ST | M. C. & FitzGerald, D. J. |
| N-cadherin | CDH2 | VEGFR2 | KDR | P. Immunotoxins. Cell |
| CD21 | CR2 | MUC18 | MCAM | 47, 641-648 (1986). |
| EGFR | EGFR | Met | MET | Gray, B. P. & Brown, K. |
| Epha2 | EPHA2 | MMP9 | MMP9 | C. Combinatorial |
| EphB4 | EPHB4 | Thomsen- | MUC1 | peptide libraries: |
| | | Friedenreich | | mining for cell-binding |
| | | carbohydrate | | peptides. Chem. Rev. |
| | | antigen | | 114, 1020-1081 |
| HER2 | ERBB2 | NRP-1 | NRP1 | (2014). |
| FGFR1 | FGFR1 | PDGFRβ | PDGFRB | Liu, R., Li, X. Xiao, W. & |
| FGFR2 | FGFR2 | CD133 | PROM1 | Lam, K. S. Tumor- |
| FGFR3 | FGFR3 | PTPRJ | PTPRJ | targeting peptides |
| FGFR4 | FGFR4 | HSPG | SDC2 | from combinatorial |
| VEGFR1 | FLT1 | E-selectin | SELE | libraries. Adv. Drug |
| VEGFR3 | FLT4 | Tie2 | TEK | Deliv. Rev. 110-111, |
| PSMA | FOLH1 | VPAC1 | VIPR1 | 13-37 (2017). |
| | | | | Bausch-Fluck, D. et al. |
| | | | | The in silico human |
| | | | | surfaceome. Proc. Natl |
| | | | | Acad. Sci. USA 115, |
| | | | | E10988-E10997 |
| | | | | (2018) |
| Other single receptor proteins that can be targeted by ligand- | | | | |
| mimicking peptides | | | | |
| Protein | Gene symbol | | | |
| ActRIIB | ACVR2B | | | |
| CD163 | CD163 | | | |
| CXCR4 | CXCR4 | | | |
| Ephrin A4 | EPHA4 | | | |
| Ephrin B1 | EPHB1 | | | |
| Ephrin B2 | EPHB2 | | | |
| Ephrin B3 | EPHB3 | | | |
| Gonadotrophin releasing | GNRHR | | | |
| hormone receptor | | | | |
| G Protein-coupled receptor 55 | GPR55 | | | |
| Bombesin receptor 2 | GRPR | | | |
| IL4 receptor | IL4R | | | |
| Low-density lipoprotein | LDLR | | | |
| receptor | | | | |
| Leptin receptor | LEPR | | | |
| LRP1 | LRP1 | | | |
| Melanocortin 1 receptor | MC1R | | | |
| Melanocortin 4 receptor | MC4R | | | |
| CD206 | MRC1 | | | |
| Urokinase plasminogen | PLAUR | | | |
| activator receptor | | | | |
| Neurokinin-1 receptor | TACR1 | | | |
| VPAC2 | VIPR2 | | | |
| Ligand-receptor repairs upregulated in lung adenocarcinoma cells | | | | |
| ligand | receptor | | | |
| LAMB1 | ITGB1 | | | Chen Z et al. Int J Biol |
| CD70 | CD27 | | | Sci. 2020; 16(12): |
| LTBP3 | ITGB5 | | | 2205-2219. |
| NID1 | ITGB1 | | | |
| COL3A1 | ITGB1 | | | |
| LAMB1 | ITGA1 | | | |
| TGM2 | ITGB1 | | | |
| CD70 | CD27 | | | |
| VCAN | ITGB1 | | | |
| COL1A1 | ITGB1 | | | |
| SERPINE2 | LRP1 | | | |
| GDF11 | ACVR2B | | | |
| VCAM1 | ITGB1 | | | |
| NID1 | COL13A1 | | | |
| JAG1 | NOTCH3 | | | |
| LTBP1 | ITGB5 | | | |
| FBLN1 | ITGB1 | | | |

| Receptor tyrosine kinases (RTKs) currently being therapeutically | |
|---|---|
| targeted in breast cancer: | |
| EGFR, VEGFR1, VEGFR2, VEGFR3, PDGFR, HER2, ErbB3, ErbB4, RET, | Butti, R et al. Mol |
| FGFR102 | Cancer 17, 34 (2018). |

In biological terms, “ligand” refers to a signaling molecule, whereas “receptor” refers to a cell membrane protein which can bind to a corresponding ligand to relay the signal downstream into the recipient cell. In the context of the disclosure, any one protein in a ligand/receptor protein pair can serve as the target-binding protein, as far as the other protein in the pair is a valuable therapeutic target. Thus by fusing a functional sequence of the target-binding protein that is directly or indirectly provided in Table 4 with a functional Fc domain, a peptibody can be constructed and engineered to be expressed and secreted in the Ab-NK cells. Upon administration, the peptibody thus secreted can selectively bind with a corresponding cell-surface target on target cells, thereby labelling these target cells, and thus the functional Fc domain of the peptibody that accumulated on the cell surface of the target cells can then recruit effector immune cells and the complement system to thereby activate the ADCC/ADCP/CDC-like cytotoxicity against these target cells.

Herein, one specific embodiment of the peptibody (i.e., anti-IL13Rα2) is provided, whose target binding domain comprises an interleukin IL13 (E13Y) ligand, or a functional fragment or variant thereof, that allows the peptibody secreted from the NK cells to selectively binds to the receptor IL13Rα2 that is found to be restrictedly expressed in the malignant glioma and renal cell carcinoma cells. Details can be found in U.S. Pat. No. 7,514,537B, which is incorporated herein by reference in its entirety. Other IL13 variants that also show strong and selective binding to the tumor-restricted IL13Rα2 receptor may include the certain residue substitutions at E13 (e.g., to Y or R), E92 (e.g., to L), K105 (e.g., to R), R109 (e.g., to K), E110, G111, R112, and F113 (these positions corresponds to the positions 11, 90, 107, 103, and 108-111 of SEQ ID NO: 21, respectively), as reported in WO2016044811A1, WO2021183960A1, and Madhankumar, A. B., et al. “Interleukin 13 mutants of enhanced avidity toward the glioma-associated receptor, IL13Rα2.” Neoplasia 6.1 (2004): 15-22 (the contents of these cited references are incorporated in this present disclosure by reference in their entirety). Thus according to different embodiments of the peptibody, the target binding domain may comprise an IL13 ligand region with one or any combinations of the above substitutions. The domain structure and the sequences of this anti-IL13Rα2 peptibody are provided in Table 5. It is expected the anti-IL13Rα2 peptibody can allow the Ab-NK cell drug that expresses the peptibody to have therapeutical effects in patients with increased IL13Rα2 expression in the tumor tissues such as the glioma or renal carcinoma.

TABLE 5
Domain and sequence information of the anti-IL13Rα2 peptibody.

| Domain | Amino Acid Sequence (SEQ ID NO) |
|---|---|
| signal peptide | MAPRRARGCRTLGLPALLLLLLLRPPATRGITAPP (SEQ ID NO: 1) |
| domain | MYRMQLLSCIALSLALVTNS (SEQ ID NO: 8; picked for the |
| | full-length construct) |
| Target-binding | GPVPPSTALRELIEELVNITQNQKAPLCNGSMVWSINLTAGMYCAALESLINVSG |
| domain | CSAIEKTQRMLSGFCPHKVSAGQFSSLHVRDTKIEVAQFVKDLLLHLKKLFREGR |
| | FN (SEQ ID NO: 21; wildtype IL13, as reference) |
| | GPVPPSTALRYLIEELVNITQNQKAPLCNGSMVWSINLTAGMYCAALESLINVSG |
| | CSAIEKTQRMLSGFCPHKVSAGQFSSLHVRDTKIEVAQFVKDLLLHLKKLFREGR |
| | FN (SEQ ID NO: 22; IL13 (E13Y, corresponding to |
| | position 11 of SEQ ID NO: 21)) |
| Linker/Hinge | GGSAEAAAKEAAAKEAAAKAGSS (SEQ ID NO: 23) |
| region | EPKSCDKTHTCP (SEQ ID NO: 16; picked for the full-length |
| | construct) |
| | GGGSGGGSGGGS (SEQ ID NO: 28) |
| Fc domain | PCPAPELLAGPDVFLLPPKPKDTLMISRTPEVTCVVVDVSFEDPEVKFNWYVDGV |
| | EVHNAKTKPPEEQYNSTLRVVSILTVLHQDWLNGKEYKCKVTNKALPLPEEKTIS |
| | KAKGQPREPQVYTLPPSRDELTKNQVSLTCLVKGFYPSDIAVEWESNGQPENNYK |
| | TTPLVLDSDGSFFLYSKLTVDKSRWQQGNVFSCSVLHEALHSHYTQKSLSLSPGK |
| | (SEQ ID NO: 24; containing the CH2 and CH3 domains, |
| | whose sequences are provided in SEQ ID NOS: 17 and 18 |
| | respectively) |
| anti-IL13Rα2 | MYRMQLLSCIALSLALVTNSGPVPPSTALRYLIEELVNITQNQKAPLCNGSMVWS |
| peptibody (full- | INLTAGMYCAALESLINVSGCSAIEKTQRMLSGFCPHKVSAGQFSSLHVRDTKIE |
| length) | VAQFVKDLLLHLKKLFREGRFNEPKSCDKTHTCPPCPAPELLAGPDVFLLPPKPK |
| | DTLMISRTPEVTCVVVDVSFEDPEVKFNWYVDGVEVHNAKTKPPEEQYNSTLRVV |
| | SILTVLHQDWLNGKEYKCKVINKALPLPEEKTISKAKGQPREPQVYTLPPSRDEL |
| | TKNQVSLTCLVKGFYPSDIAVEWESNGQPENNYKTTPLVLDSDGSFFLYSKLTVD |
| | KSRWQQGNVFSCSVLHEALHSHYTQKSLSLSPGK (SEQ ID NO: 25; |
| | derived from SEQ ID NOS: 8, 22, 16, and 24) |

In addition to the above natural ligands, the peptibody may comprise an artificial peptide sequence, derived from a peptide that has been computer-predicted and experimentally screened (e.g., in a phage display) to display a strong and selective binding activity to a target molecule on the cell surface of a target cell.

Furthermore, in order to reduce the off-target effect, a masking peptide can be similarly fused with the peptibody by means of a degradable linker. Details for such design can be found in the relevant discussions for the masking peptide for the antibody as provided above.

Herein, it is to be noted that in addition to NK cells, other immune cells such as lymphocytes, T cells, and tumor-infiltrating lymphocyte (TILs) etc., can also be engineered to express these above specific antibodies or peptibodies, which thus may also equip these engineered immune cells with an additional ADCC/ADCP/CDC activities.

The following are noted for the above two NK cell drug platforms.
1) In addition to the immune effector functionalities such as ADCC/ADCP/CDC activities or alike, the antibody or the peptibody that is engineered to be expressed and secreted in the Ab-NK cells, the antibody/peptibody can also exert other functionalities including neutralizing certain agents, blocking, and/or agonizing certain signal pathways, etc.
2) According to some embodiments, the antibody/peptibody construct may be engineered as multivalent, comprising at least two target-binding domains (i.e., an antigen-binding domain can also be regarded as a target-binding domain) fused with one single functional Fc domain. Once expressed and secreted, such multivalent target-binding protein can recognize at least two different cell-surface target molecules, thereby one single Ab-NK cell drug obtained thereby may allow the elimination of target cells expressing multiple tumor targets or multiple different types of target cells (one tumor cell and one stromal cells in the tumor issues) in one subject administered with the Ab-NK cells, causing a better therapeutic effect.
3) Optionally, in order to boost the antibody production, the NK cells can optionally be configured to co-express a secreted protein production booster such as SRP14 and other signal recognition particle proteins and protein members of the translocon complex, such as SEC61A, TRAP complex and glycosyltransferase.
4) In any of the NK cell drug platforms as described above, the NK cells can be of any source. According to some preferred embodiments, the NK cells can be obtained from cord blood, which provide cells that are easy to expand and be engineered in vitro. Other sources may include peripheral blood NK cells, iPSC derived NK cells or NK cell lines such as NK-92.
5) In order to establish any or both of the above mentioned two NK cell drug platforms, at least one of the above mentioned transgenes can be transduced into the NK cells obtained from any source described above. Briefly, the transgene(s) may be inserted into the NK cell genome using all the available genetic engineering approaches, such as CRISPR/Cas9, TALEN, zinc finger proteins, recombinase-mediated gene targeting or viral vector. The expression of one or more transgenes are required to obtain the NK cell drugs. If a retroviral vector is used, optionally and preferably, the retroviral vector is modified (e.g., with mutated WPRE) for enhanced clinical safety. If more than one transgene is to be transduced, these transgenes may optionally be in a same vector or in different vectors, and if in the same vector, may optionally be transcribed and translated in different transcripts, or may optionally be transcribed and translated in a single polypeptide, separated by a 2A self-cleaving peptide (“2A”). It is noted that there can be other different embodiments regarding the above mentioned transgene construction and transduction methods.

In one aspect, the disclosure is related to an engineered immune cell, expressing and/or secreting a fusion polypeptide comprising, optionally from N-terminus to C-terminus: a first moiety comprising a ligand polypeptide that is capable of binding to a receptor expressed on the surface of cancer cells (e.g., IL13Rα2 or any of the receptors in Table 4), and a second moiety comprising an fragment crystallizable (Fc) region. In some embodiments, the immune cell expresses an Fc receptor (e.g., CD16) on the cell surface that can recognize the Fc region of the fusion polypeptide and mediate antibody-dependent cellular cytotoxicity (ADCC) for the engineered immune cell. In some embodiments, the immune cell is a natural killer (NK) cell (e.g., a NK cell obtained from cord blood), a T cell, or a tumor-infiltrating cell (TIL). In some embodiments, the ligand polypeptide comprises or consists of a wildtype human IL-13 (SEQ ID NO: 21). In some embodiments, the human IL-13 comprises a Tyr residue at a position corresponding to Glu11 of SEQ ID NO: 21. In some embodiments, the Fc region comprises the CH2 and CH3 domains of human IgG (e.g., IgG1). In some embodiments, the ligand polypeptide comprises an amino acid sequence that is at least 80%, 85%, 90%, 95%, 96%, 97%, 98%, 99% or 100% identical to SEQ ID NO: 21 or 22, and the Fc region comprises an amino acid sequence that is at least 80%, 85%, 90%, 95%, 96%, 97%, 98%, 99% or 100% identical to SEQ ID NO: 17, 18, or 24. In some embodiments, the Fc region includes any of the substitutions or mutations described herein (e.g., any of the mutations in FIG. 4). In some embodiments, the ligand polypeptide and the Fc region are fused via a linker peptide (e.g., a flexible linker) or a hinge region, wherein the linker peptide or the hinge region comprises an amino acid sequence that is at least 80%, 85%, 90%, 95%, 96%, 97%, 98%, 99% or 100% identical to SEQ ID NO: 16, 23, or 28. In some embodiments, the fusion polypeptide further comprises an N-terminal signal peptide. In some embodiments, the signal peptide comprises or consists of a sequence corresponding to amino acids 1-20 of human IL-2 protein (NCBI reference number: NP_000577.2). In some embodiments, the signal peptide is the signal peptide of human IL-2. In some embodiments, the signal peptide comprises or consists of a sequence corresponding to amino acids 1-35 of human IL-15Rα protein (NCBI reference number: NP_002180.1), optionally the signal peptide comprises an Ala residue at a position corresponding to Cys33 of human IL-15Rα protein. In some embodiments, the signal peptide comprises the signal peptide of human IL-15Rα protein. In some embodiments, the signal peptide comprises an amino acid sequence that is at least 80%, 85%, 90%, 95%, 96%, 97%, 98%, 99% or 100% identical to SEQ ID NO: 1 or 8. In some embodiments, the fusion polypeptide comprises an amino acid sequence that is at least 80%, 85%, 90%, 95%, 96%, 97%, 98%, 99% or 100% identical to SEQ ID NO: 25. In some embodiments, the engineered immune cell described herein further expresses another fusion polypeptide comprising, optionally from N-terminus to C-terminus: a first moiety comprising all or a portion of interleukin 15 (IL15), and a second moiety comprising all or a portion of IL15Rα, in some embodiments, the fusion polypeptide can form a functional complex with IL15Rβ and IL15Rγ to activate IL-15 signaling in the engineered immune cell.

### Antibodies and Antigen-Binding Fragments

The disclosure provides antibodies and antigen-binding fragments thereof that specifically bind to PD-L1 (e.g., human PD-L1). The antibodies and antigen-binding fragments described herein are capable of binding to PD-L1. In some embodiments, these antibodies can block the binding of human PD-L1 to PD-L1 ligand (e.g., PD-1). In some embodiments, these antibodies can initiate ADCC, ADCP, and/or CDC activities. In some embodiments, these antibodies bind to cells (e.g., cancer cells) expressing PD-L1.

The disclosure provides, e.g., anti-PD-L1 antibodies L8 and L2, the modified antibodies thereof, including, e.g., chimeric antibodies, humanized antibodies, and human antibodies.

The CDR sequences for L2, and L2-derived antibodies (e.g., humanized antibodies) include CDRs of the heavy chain variable domain, SEQ ID NOs: 31, 32, 33, and CDRs of the light chain variable domain, SEQ ID NOs: 34, 35, 36, as defined by Kabat definition. The CDRs can also be defined by North/Aho system. Under the North/Aho definition, the CDR sequences of the heavy chain variable domain are set forth in SEQ ID NOs: 43, 44, 45, and CDR sequences of the light chain variable domain are set forth in SEQ ID NOs: 46, 47, 48.

The CDR sequences for L8, and L8-derived antibodies (e.g., humanized antibodies) include CDRs of the heavy chain variable domain, SEQ ID NOs: 37, 38, 39, and CDRs of the light chain variable domain, SEQ ID NOs: 40, 41, 42, as defined by Kabat definition. The CDRs can also be defined by North/Aho system. Under the North/Aho definition, the CDR sequences of the heavy chain variable domain are set forth in SEQ ID NOs: 49, 50, 51, and CDR sequences of the light chain variable domain are set forth in SEQ ID NOs: 52, 53, 54.

The amino acid sequence for the heavy chain variable region of L8 antibody is set forth in SEQ ID NO: 10. The amino acid sequence for the light chain variable region of L8 antibody is set forth in SEQ ID NO: 9. The amino acid sequence for the heavy chain variable region of L2 antibody is set forth in SEQ ID NO: 14. The amino acid sequence for the light chain variable region of L2 antibody is set forth in SEQ ID NO: 13.

The amino acid sequences for heavy chain variable regions and light variable regions of the modified antibodies are also provided. In some embodiments, the heavy chain variable region is at least 80%, 85%, 90%, 91%, 92%, 93%, 94%, 95%, 96%, 97%, 98%, or 99% identical to SEQ ID NO: 10 or 14. In some embodiments, the light chain variable region is at least 80%, 85%, 90%, 91%, 92%, 93%, 94%, 95%, 96%, 97%, 98%, or 99% identical to SEQ ID NO: 9 or 13. The heavy chain variable region sequence can be paired with the corresponding light chain variable region sequence, and together they bind to PD-L1.

Furthermore, in some embodiments, the antibodies or antigen-binding fragments thereof described herein can also contain one, two, or three heavy chain variable region CDRs selected from the group of SEQ ID NOs: 31-33, SEQ ID NOs: 37-39, SEQ ID NOs: 43-45, and SEQ ID NOs: 49-51; and/or one, two, or three light chain variable region CDRs selected from the group of SEQ ID NOs: 34-36, SEQ ID NOs: 40-42, SEQ ID NOs: 46-48, and SEQ ID NOs: 52-54.

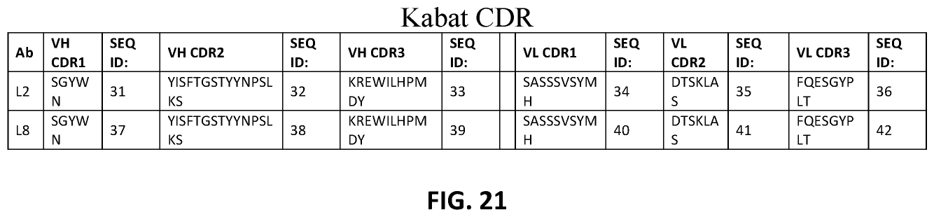

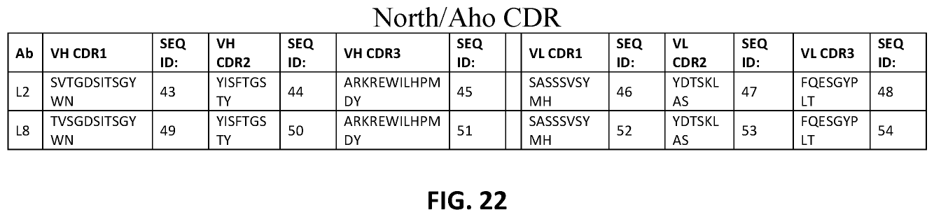

In some embodiments, the antibodies can have a heavy chain variable region (VH) comprising complementarity determining regions (CDRs) 1, 2, 3, wherein the CDR1 region comprises or consists of an amino acid sequence that is at least 80%, 85%, 90%, or 95% identical to a selected VH CDR1 amino acid sequence, the CDR2 region comprises or consists of an amino acid sequence that is at least 80%, 85%, 90%, or 95% identical to a selected VH CDR2 amino acid sequence, and the CDR3 region comprises or consists of an amino acid sequence that is at least 80%, 85%, 90%, or 95% identical to a selected VH CDR3 amino acid sequence. In some embodiments, the antibodies can have a light chain variable region (VL) comprising CDRs 1, 2, 3, wherein the CDR1 region comprises or consists of an amino acid sequence that is at least 80%, 85%, 90%, or 95% identical to a selected VL CDR1 amino acid sequence, the CDR2 region comprises or consists of an amino acid sequence that is at least 80%, 85%, 90%, or 95% identical to a selected VL CDR2 amino acid sequence, and the CDR3 region comprises or consists of an amino acid sequence that is at least 80%, 85%, 90%, or 95% identical to a selected VL CDR3 amino acid sequence. The selected VH CDRs 1, 2, 3 amino acid sequences and the selected VL CDRs, 1, 2, 3 amino acid sequences are shown in FIG. 21 (CDRs under Kabat definition), and FIG. 22 (CDRs under North/Aho definition).

In some embodiments, the antibody or an antigen-binding fragment described herein can contain a heavy chain variable domain containing one, two, or three of the CDRs of SEQ ID NO: 31 with zero, one or two amino acid insertions, deletions, or substitutions; SEQ ID NO: 32 with zero, one or two amino acid insertions, deletions, or substitutions; SEQ ID NO: 33 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 37 with zero, one or two amino acid insertions, deletions, or substitutions; SEQ ID NO: 38 with zero, one or two amino acid insertions, deletions, or substitutions; SEQ ID NO: 39 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 43 with zero, one or two amino acid insertions, deletions, or substitutions; SEQ ID NO: 44 with zero, one or two amino acid insertions, deletions, or substitutions; SEQ ID NO: 45 with zero, one or two amino acid insertions, deletions, or substitutions.

In some embodiments, the antibody or an antigen-binding fragment described herein can contain a heavy chain variable domain containing one, two, or three of the CDRs of SEQ ID NO: 49 with zero, one or two amino acid insertions, deletions, or substitutions; SEQ ID NO: 50 with zero, one or two amino acid insertions, deletions, or substitutions; SEQ ID NO: 51 with zero, one or two amino acid insertions, deletions, or substitutions.

In some embodiments, the antibody or an antigen-binding fragment described herein can contain a light chain variable domain containing one, two, or three of the CDRs of SEQ ID NO: 34 with zero, one or two amino acid insertions, deletions, or substitutions; SEQ ID NO: 35 with zero, one or two amino acid insertions, deletions, or substitutions; SEQ ID NO: 36 with zero, one or two amino acid insertions, deletions, or substitutions.

In some embodiments, the antibody or an antigen-binding fragment described herein can contain a light chain variable domain containing one, two, or three of the CDRs of SEQ ID NO: 40 with zero, one or two amino acid insertions, deletions, or substitutions; SEQ ID NO: 41 with zero, one or two amino acid insertions, deletions, or substitutions; SEQ ID NO: 42 with zero, one or two amino acid insertions, deletions, or substitutions.

In some embodiments, the antibody or an antigen-binding fragment described herein can contain a light chain variable domain containing one, two, or three of the CDRs of SEQ ID NO: 46 with zero, one or two amino acid insertions, deletions, or substitutions; SEQ ID NO: 47 with zero, one or two amino acid insertions, deletions, or substitutions; SEQ ID NO: 48 with zero, one or two amino acid insertions, deletions, or substitutions.

In some embodiments, the antibody or an antigen-binding fragment described herein can contain a light chain variable domain containing one, two, or three of the CDRs of SEQ ID NO: 52 with zero, one or two amino acid insertions, deletions, or substitutions; SEQ ID NO: 53 with zero, one or two amino acid insertions, deletions, or substitutions; SEQ ID NO: 54 with zero, one or two amino acid insertions, deletions, or substitutions.

The insertions, deletions, and substitutions can be within the CDR sequence, or at one or both terminal ends of the CDR sequence. In some embodiments, the CDR is determined based on Kabat definition scheme. In some embodiments, the CDR is determined based on North/Aho definition scheme. In some embodiments, the CDR is determined based on a combination of Kabat and North/Aho definition scheme. In some embodiments, the CDR is determined based on IMGT definition. In some embodiments, the CDR is determined based on contact definition. In some embodiments, the CDR is determined based on Chothia definition. In some embodiments, the CDR is determined using computer tool ABodyBuilder2 (details can be found at https://opig.stats.ox.ac.uk/webapps/sabdab-sabpred/sabpred).

The disclosure also provides antibodies or antigen-binding fragments thereof that bind to PD-L1. The antibodies or antigen-binding fragments thereof contain a heavy chain variable region (VH) comprising or consisting of an amino acid sequence that is at least 80%, 85%, 90%, or 95% identical to a selected VH sequence, and a light chain variable region (VL) comprising or consisting of an amino acid sequence that is at least 80%, 85%, 90%, or 95% identical to a selected VL sequence. In some embodiments, the selected VH sequence is SEQ ID NO: 10, and the selected VL sequence is SEQ ID NO: 9. In some embodiments, the selected VH sequence is SEQ ID NO: 14 and the selected VL sequence is SEQ ID NO: 13.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences. For example, the comparison of sequences and determination of percent identity between two sequences can be accomplished using a Blossum 62 scoring matrix with a gap penalty of 12, a gap extend penalty of 4, and a frameshift gap penalty of 5.

The disclosure also provides nucleic acid comprising a polynucleotide encoding a polypeptide comprising an immunoglobulin heavy chain or an immunoglobulin light chain. The immunoglobulin heavy chain or immunoglobulin light chain comprises CDRs as shown in FIG. 21, or FIG. 22, or VH/VL shown in Table 3. When the polypeptides are paired with corresponding polypeptide (e.g., a corresponding heavy chain variable region or a corresponding light chain variable region), the paired polypeptides bind to PD-L1.

The anti-PD-L1 antibodies and antigen-binding fragments can also be antibody variants (including derivatives and conjugates) of antibodies or antibody fragments and multi-specific (e.g., bi-specific) antibodies or antibody fragments. Additional antibodies provided herein are polyclonal, monoclonal, multimeric, multi-specific (e.g., bi-specific), human antibodies, chimeric antibodies (e.g., human-mouse chimera), single-chain antibodies, intracellularly-made antibodies (i.e., intrabodies), and antigen-binding fragments thereof. The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass. In some embodiments, the antibody or antigen-binding fragment thereof is an IgG antibody or antigen-binding fragment thereof.

Fragments of antibodies are suitable for use in the methods provided so long as they retain the desired affinity and specificity of the full-length antibody. Thus, a fragment of an antibody that binds to PD-L1 will retain an ability to bind to PD-L1. An Fv fragment is an antibody fragment which contains a complete antigen recognition and binding site. This region consists of a dimer of one heavy and one light chain variable domain in tight association, which can be covalent in nature, for example in scFv. It is in this configuration that the three CDRs of each variable domain interact to define an antigen binding site on the surface of the VH-VL dimer. Collectively, the six CDRs or a subset thereof confer antigen binding specificity to the antibody. However, even a single variable domain (or half of an Fv comprising only three CDRs specific for an antigen) can have the ability to recognize and bind antigen, although usually at a lower affinity than the entire binding site. Single-chain Fv or (scFv) antibody fragments comprise the VH and VL domains (or regions) of antibody, wherein these domains are present in a single polypeptide chain. Generally, the scFv polypeptide further comprises a polypeptide linker between the VH and VL domains, which enables the scFv to form the desired structure for antigen binding.

The present disclosure also provides an antibody or antigen-binding fragment thereof that cross-competes with any antibody or antigen-binding fragment as described herein. The cross-competing assay is known in the art, and is described e.g., in Moore et al., “Antibody cross-competition analysis of the human immunodeficiency virus type 1 gp120 exterior envelope glycoprotein.” Journal of Virology 70.3 (1996): 1863-1872, which is incorporated herein reference in its entirety. In one aspect, the present disclosure also provides an antibody or antigen-binding fragment thereof that binds to the same epitope or region as any antibody or antigen-binding fragment as described herein. The epitope binning assay is known in the art, and is described e.g., in Estep et al. “High throughput solution-based measurement of antibody-antigen affinity and epitope binning.” MAbs. Vol. 5. No. 2. Taylor & Francis, 2013, which is incorporated herein reference in its entirety.

The present disclosure provides various antibodies and antigen-binding fragments thereof derived from anti-PD-L1 antibodies described herein. In general, antibodies (also called immunoglobulins) are made up of two classes of polypeptide chains, light chains and heavy chains. A non-limiting examples of antibody of the present disclosure can be an intact, four immunoglobulin chain antibody comprising two heavy chains and two light chains. The heavy chain of the antibody can be of any isotype including IgM, IgG, IgE, IgA, or IgD or sub-isotype including IgG1, IgG2, IgG2a, IgG2b, IgG3, IgG4, IgE1, IgE2, etc. The light chain can be a kappa light chain or a lambda light chain. An antibody can comprise two identical copies of a light chain and two identical copies of a heavy chain. The heavy chains, which each contain one variable domain (or variable region, VH) and multiple constant domains (or constant regions), bind to one another via disulfide bonding within their constant domains to form the “stem” of the antibody. The light chains, which each contain one variable domain (or variable region, VL) and one constant domain (or constant region), each bind to one heavy chain via disulfide binding. The variable region of each light chain is aligned with the variable region of the heavy chain to which it is bound. The variable regions of both the light chains and heavy chains contain three hypervariable regions sandwiched between more conserved framework regions (FR).

These hypervariable regions, known as the complementary determining regions (CDRs), form loops that comprise the antigen binding surface of the antibody. The four framework regions largely adopt a beta-sheet conformation and the CDRs form loops connecting the beta-sheet structure, and in some cases forming part of, the beta-sheet structure. The CDRs in each chain are held in close proximity by the framework regions and, with the CDRs from the other chain, contribute to the formation of the antigen-binding region.

Methods for identifying the CDR regions of an antibody by analyzing the amino acid sequence of the antibody are well known, and a number of definitions of the CDRs are commonly used. The Kabat definition is based on sequence variability, and the Chothia definition is based on the location of the structural loop regions. These methods and definitions are described in, e.g., Martin, “Protein sequence and structure analysis of antibody variable domains,” Antibody engineering, Springer Berlin Heidelberg, 2001. 422-439; Abhinandan, et al. “Analysis and improvements to Kabat and structurally correct numbering of antibody variable domains,” Molecular immunology 45.14 (2008): 3832-3839; Wu, T. T. and Kabat, E. A. (1970) J. Exp. Med. 132:211-250; Martin et al., Methods Enzymol. 203:121-53 (1991); Morea et al., Biophys Chem. 68 (1-3): 9-16 (Oct. 1997); Morea et al., J Mol Biol. 275 (2): 269-94 (Jan. 1998); Chothia et al., Nature 342 (6252): 877-83 (Dec. 1989); Ponomarenko and Bourne, BMC Structural Biology 7:64 (2007); each of which is incorporated herein by reference in its entirety.

The CDRs are important for recognizing an epitope of an antigen. As used herein, an “epitope” is the smallest portion of a target molecule capable of being specifically bound by the antigen binding domain of an antibody. The minimal size of an epitope may be about three, four, five, six, or seven amino acids, but these amino acids need not be in a consecutive linear sequence of the antigen's primary structure, as the epitope may depend on an antigen's three-dimensional configuration based on the antigen's secondary and tertiary structure.

In some embodiments, the antibody is an intact immunoglobulin molecule (e.g., IgG1, IgG2a, IgG2b, IgG3, IgM, IgD, IgE, IgA). The IgG subclasses (IgG1, IgG2, IgG3, and IgG4) are highly conserved, differ in their constant region, particularly in their hinges and upper CH2 domains. The sequences and differences of the IgG subclasses are known in the art, and are described, e.g., in Vidarsson, et al, “IgG subclasses and allotypes: from structure to effector functions.” Frontiers in immunology 5 (2014); Irani, et al. “Molecular properties of human IgG subclasses and their implications for designing therapeutic monoclonal antibodies against infectious diseases.” Molecular immunology 67.2 (2015): 171-182; Shakib, Farouk, ed. The human IgG subclasses: molecular analysis of structure, function and regulation. Elsevier, 2016; each of which is incorporated herein by reference in its entirety.

The antibody can also be an immunoglobulin molecule that is derived from any species (e.g., human, rodent, mouse, camelid). Antibodies disclosed herein also include, but are not limited to, polyclonal, monoclonal, monospecific, polyspecific antibodies, and chimeric antibodies that include an immunoglobulin binding domain fused to another polypeptide. The term “antigen binding domain” or “antigen binding fragment” is a portion of an antibody that retains specific binding activity of the intact antibody, i.e., any portion of an antibody that is capable of specific binding to an epitope on the intact antibody's target molecule. It includes, e.g., Fab, Fab′, F(ab′) 2, and variants of these fragments. Thus, in some embodiments, an antibody or an antigen binding fragment thereof can be, e.g., a scFv, a Fv, a Fd, a dAb, a bispecific antibody, a bispecific scFv, a diabody, a linear antibody, a single-chain antibody molecule, a multi-specific antibody formed from antibody fragments, and any polypeptide that includes a binding domain which is, or is homologous to, an antibody binding domain. Non-limiting examples of antigen binding domains include, e.g., the heavy chain and/or light chain CDRs of an intact antibody, the heavy and/or light chain variable regions of an intact antibody, full length heavy or light chains of an intact antibody, or an individual CDR from either the heavy chain or the light chain of an intact antibody.

Fragments of antibodies are suitable for use in the methods described herein are also provided. The Fab fragment contains a variable and constant domain of the light chain and a variable domain and the first constant domain (CH1) of the heavy chain. F(ab′) 2 antibody fragments comprise a pair of Fab fragments which are generally covalently linked near their carboxy termini by hinge cysteines between them. Other chemical couplings of antibody fragments are also known in the art.

Diabodies are small antibody fragments with two antigen-binding sites, which fragments comprise a VH connected to a VL in the same polypeptide chain (VH and VL). By using a linker that is too short to allow pairing between the two domains on the same chain, the domains are forced to pair with the complementary domains of another chain and create two antigen-binding sites.

Linear antibodies comprise a pair of tandem Fd segments (VH-CH1-VH-CH1) which, together with complementary light chain polypeptides, form a pair of antigen binding regions. Linear antibodies can be bispecific or monospecific.

Antibodies and antibody fragments of the present disclosure can be modified in the Fc region to provide desired effector functions or serum half-life.

Multimerization of antibodies may be accomplished through natural aggregation of antibodies or through chemical or recombinant linking techniques known in the art. For example, some percentage of purified antibody preparations (e.g., purified IgG1 molecules) spontaneously form protein aggregates containing antibody homodimers and other higher-order antibody multimers.

Alternatively, antibody homodimers may be formed through chemical linkage techniques known in the art. For example, heterobifunctional crosslinking agents including, but not limited to SMCC (succinimidyl 4-(maleimidomethyl)cyclohexane-1-carboxylate) and SATA (N-succinimidyl S-acethylthio-acetate) can be used to form antibody multimers. An exemplary protocol for the formation of antibody homodimers is described in Ghetie et al. (Proc. Natl. Acad. Sci. U.S.A. 94:7509-7514, 1997). Antibody homodimers can be converted to Fab′2 homodimers through digestion with pepsin. Another way to form antibody homodimers is through the use of the autophilic T15 peptide described in Zhao et al. (J. Immunol. 25:396-404, 2002).

In some embodiments, the multi-specific antibody is a bi-specific antibody. Bi-specific antibodies can be made by engineering the interface between a pair of antibody molecules to maximize the percentage of heterodimers that are recovered from recombinant cell culture. For example, the interface can contain at least a part of the CH3 domain of an antibody constant domain. In this method, one or more small amino acid side chains from the interface of the first antibody molecule are replaced with larger side chains (e.g., tyrosine or tryptophan). Compensatory “cavities” of identical or similar size to the large side chain(s) are created on the interface of the second antibody molecule by replacing large amino acid side chains with smaller ones (e.g., alanine or threonine). This provides a mechanism for increasing the yield of the heterodimer over other unwanted end-products such as homodimers. This method is described, e.g., in WO 96/27011, which is incorporated by reference in its entirety.

Bi-specific antibodies include cross-linked or “heteroconjugate” antibodies. For example, one of the antibodies in the heteroconjugate can be coupled to avidin and the other to biotin. Heteroconjugate antibodies can also be made using any convenient cross-linking methods. Suitable cross-linking agents and cross-linking techniques are well known in the art and are disclosed in U.S. Pat. No. 4,676,980, which is incorporated herein by reference in its entirety.

Any of the antibodies or antigen-binding fragments described herein may be conjugated to a stabilizing molecule (e.g., a molecule that increases the half-life of the antibody or antigen-binding fragment thereof in a subject or in solution). Non-limiting examples of stabilizing molecules include: a polymer (e.g., a polyethylene glycol) or a protein (e.g., serum albumin, such as human serum albumin). The conjugation of a stabilizing molecule can increase the half-life or extend the biological activity of an antibody or an antigen-binding fragment in vitro (e.g., in tissue culture or when stored as a pharmaceutical composition) or in vivo (e.g., in a human).

In some embodiments, the antibodies or antigen-binding fragments described herein can be conjugated to a therapeutic agent. The antibody-drug conjugate comprising the antibody or antigen-binding fragment thereof can covalently or non-covalently bind to a therapeutic agent. In some embodiments, the therapeutic agent is a cytotoxic or cytostatic agent (e.g., cytochalasin B, gramicidin D, ethidium bromide, emetine, mitomycin, etoposide, teniposide, vincristine, vinblastine, colchicin, doxorubicin, daunorubicin, dihydroxyanthracin, maytansinoids such as DM-1 and DM-4, dione, mitoxantrone, mithramycin, actinomycin D, 1-dehydrotestosterone, glucocorticoids, procaine, tetracaine, lidocaine, propranolol, puromycin, epirubicin, and cyclophosphamide and analogs).

In some embodiments, the antigen binding fragment can form a part of a chimeric antigen receptor (CAR). In some embodiments, the chimeric antigen receptor are fusions of single-chain variable fragments (scFv) as described herein, fused to CD3-zeta transmembrane- and endodomain. In some embodiments, the chimeric antigen receptor also comprises intracellular signaling domains from various costimulatory protein receptors (e.g., CD28, 41BB, ICOS). In some embodiments, the chimeric antigen receptor comprises multiple signaling domains, e.g., CD3z-CD28-41BB or CD3z-CD28-OX40, to increase potency. Thus, in one aspect, the disclosure further provides cells (e.g., T cells) that express the chimeric antigen receptors as described herein.

In some embodiments, the scFv has one heavy chain variable domain, and one light chain variable domain. In some embodiments, the scFv has two heavy chain variable domains, and two light chain variable domains.

In some embodiments, sequences (e.g., CDRs or VH/VL sequences) of the antibody or antigen-binding fragment thereof described herein can be used to generate a bispecific antibody targeting PD-L1 and an addition antigen.

In some embodiments, the anti-PD-L1 antibodies or antigen-binding fragments thereof described herein can bind to cells expressing PD-L1 with a Kd value less than about 0.1 μg/mL, less than about 0.09 μg/mL, less than about 0.08 μg/mL, less than about 0.07 μg/mL, less than about 0.06 μg/mL, less than about 0.05 μg/mL, less than about 0.04 μg/mL, or less than about 0.03 μg/mL. In some embodiments, the anti-PD-L1 antibodies or antigen-binding fragments thereof described herein can bind to cells expressing PD-L1 with a comparable Kd value (less than about 120%, less than about 150%, less than about 180%, less than about 200%, less than about 250%, or less than about 300%) as compared to a reference anti-PD-L1 antibody (e.g., atezolizumab).

In some embodiments, the anti-PD-L1 antibodies or antigen-binding fragments thereof described herein can bind to recombinant human PD-L1 protein with a Kd value less than about 0.1 μg/mL, less than about 0.09 μg/mL, less than about 0.08 μg/mL, less than about 0.07 μg/mL, less than about 0.06 μg/mL, less than about 0.05 μg/mL, less than about 0.04 μg/mL, less than about 0.03 μg/mL, less than about 0.02 μg/mL, or less than about 0.01 μg/mL. In some embodiments, the anti-PD-L1 antibodies or antigen-binding fragments thereof described herein can bind to recombinant human PD-L1 protein with a comparable Kd value (less than about 120%, less than about 150%, less than about 180%, less than about 200%, less than about 250%, less than about 300%, less than about 340%, less than about 400%, less than about 450%, or less than about 500%) as compared to a reference anti-PD-L1 antibody (e.g., atezolizumab).

In some embodiments, the anti-PD-L1 antibodies or antigen-binding fragments thereof described herein can block the interaction between PD-1 and PD-L1 with an EC₅₀ value of less than about 0.1 μg/mL, less than about 0.09 μg/mL, less than about 0.08 μg/mL, less than about 0.07 μg/mL, less than about 0.06 μg/mL, less than about 0.05 μg/mL, less than about 0.04 μg/mL, less than about 0.03 μg/mL, less than about 0.02 μg/mL, or less than about 0.01 μg/mL. In some embodiments, the anti-PD-L1 antibodies or antigen-binding fragments thereof described herein can block the interaction between PD-1 and PD-L1 with a comparable Kd value (less than about 120%, less than about 150%, less than about 180%, less than about 200%, less than about 250%, or less than about 300%) as compared to a reference anti-PD-L1 antibody (e.g., atezolizumab).

In some embodiments, the anti-PD-L1 antibodies or antigen-binding fragments thereof described herein can specifically bind to PD-L1. In some embodiments, the anti-PD-L1 antibodies or antigen-binding fragments thereof described herein does not specifically bind to SSTR4 or FCGR proteins (e.g., FCGR1A, FCGR3B, or FCGR2B).

### Engineered Cells

The present disclosure provides engineered cells (e.g., NK cells) that express a cell membrane-bound IL15 (e.g., any of the membrane-bound IL15 constructs described herein), and/or express/secret an Fc-containing antibodies or antigen-binding fragments thereof and/or peptibodies (e.g., any of the antibody or antigen-binding fragment thereof described herein, or any of the peptibodies described herein). These engineered cells can be used to treat various disorders or disease as described herein (e.g., cancers).

In various embodiments, the cell that is engineered can be obtained from e.g., humans and non-human animals. In various embodiments, the cell that is engineered can be obtained from bacteria, fungi, humans, rats, mice, rabbits, monkeys, pig or any other species. Preferably, the cell is from humans, rats or mice. More preferably, the cell is obtained from humans. In various embodiments, the cell that is engineered is a blood cell. Preferably, the cell is a leukocyte, lymphocyte or any other suitable blood cell type. In some embodiments, the cell is a peripheral blood cell. In some embodiments, the cell is a T cell, a tumor-infiltrating cell (TIL), or an NK cell. In some embodiments, the cell is an immune cell, e.g., a cord blood-derived NK cell.

In some embodiments, the cell is an NK cell. In some embodiments, preparation of the engineered cells includes one or more culture and/or preparation steps. The cells for engineering to express the chimeric polypeptides (or the fusion polypeptide), antibodies or antigen-binding fragments thereof, and/or peptibodies, can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

In some embodiments, the cell is a T cell. In some embodiments, the T cells can express a cell surface receptor that recognizes a specific antigenic moiety on the surface of a target cell. The cell surface receptor can be a wild type or recombinant T cell receptor (TCR), a chimeric antigen receptor (CAR), or any other surface receptor capable of recognizing an antigenic moiety that is associated with the target cell. T cells can be obtained by various methods known in the art, e.g., in vitro culture of T cells (e.g., tumor infiltrating lymphocytes) isolated from patients. TCR gene-modified T cells can be obtained by transducing T cells (e.g., isolated from the peripheral blood of patients), with a viral vector. In some embodiments, the T cell is a TCR gene-modified T cell. In some embodiments, the T cells are CD4+ T cells, CD8+ T cells, or regulatory T cells. In some embodiments, the T cells are T helper type 1 T cells and T helper type 2 T cells. In some embodiments, the T cell expressing this receptor is an αβ-T cell. In alternate embodiments, the T cell expressing this receptor is a γδ-T cell.

In some embodiments, the cells are stem cells, such as multipotent and pluripotent stem cells, including induced pluripotent stem cells (iPSCs). The cells can be primary cells, such as those isolated directly from a subject and/or isolated from a subject and frozen. In some embodiments, the stem cells are cultured with additional differentiation factors to obtain desired cell types (e.g., NK cells).

Different cell types can be obtained from appropriate isolation methods. The isolation methods include the separation of different cell types based on the expression or presence in the cell of one or more specific molecules, such as surface markers, e.g., surface proteins, intracellular markers, or nucleic acid. In some embodiments, any known method for separation based on such markers can be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be particularly useful where no antibody is available that specifically identifies a cell type in a heterogeneous population, such that separation is best carried out based on markers expressed by cells other than the desired population.

Also provided are methods, nucleic acids, compositions, and kits, for expressing the chimeric polypeptides (or the fusion polypeptide), antibodies or antigen-binding fragments thereof, and/or peptibodies described herein, and for producing the genetically engineered immune cells (e.g., NK cells) expressing such molecules. The genetic engineering generally involves introduction of a nucleic acid encoding these molecules into the cell, such as by retroviral transduction, transfection, or transformation. In some embodiments, gene transfer is accomplished by first stimulating the cell, such as by combining it with a stimulus that induces a response such as proliferation, survival, and/or activation, e.g., as measured by expression of a cytokine or activation marker, followed by transduction of the activated cells, and expansion in culture to numbers sufficient for clinical application.

In some embodiments, recombinant nucleic acids are transferred into cells using recombinant infectious virus particles, such as, e.g., vectors derived from simian virus 40 (SV40), adenoviruses, adeno-associated virus (AAV). In some embodiments, recombinant nucleic acids are transferred into T cells using recombinant lentiviral vectors or retroviral vectors, such as gamma-retroviral vectors. In some embodiments, the retroviral vector has a long terminal repeat sequence (LTR), e.g., a retroviral vector derived from the Moloney murine leukemia virus (MoMLV), myeloproliferative sarcoma virus (MPSV), murine embryonic stem cell virus (MESV), murine stem cell virus (MSCV), or spleen focus forming virus (SFFV). Most retroviral vectors are derived from murine retroviruses. In some embodiments, the retroviruses include those derived from any avian or mammalian cell source. The retroviruses typically are amphotropic, meaning that they are capable of infecting host cells of several species, including humans. In some embodiments, the vector is a lentivirus vector. In some embodiments, recombinant nucleic acids are transferred into T cells via electroporation. In some embodiments, recombinant nucleic acids are transferred into T cells via transposition. Other methods of introducing and expressing genetic material in immune cells include calcium phosphate transfection, protoplast fusion, cationic liposome-mediated transfection; tungsten particle-facilitated microparticle bombardment and strontium phosphate DNA co-precipitation. Many of these methods are descried e.g., in WO2019195486, which is incorporated herein by reference in its entirety.

Also provided are populations of engineered cells, compositions containing such cells and/or enriched for such cells, such as in which cells expressing the binding molecule make up at least 15%, 20%, 25%, 30%, 35%, 40%, 50%, 60%, 70%, 80%, 90%, 91%, 92%, 93%, 94%, 95%, 96%, 97%, 98%, 99%, or more percent of the total cells in the composition or cells of a certain type such as NK cells, T cells, or TILs.

### Recombinant Vectors

The present disclosure also provides recombinant vectors (e.g., an expression vectors) that include an isolated polynucleotide disclosed herein (e.g., a polynucleotide that encodes a polypeptide disclosed herein), host cells into which are introduced the recombinant vectors (i.e., such that the host cells contain the polynucleotide and/or a vector comprising the polynucleotide), and the production of recombinant polypeptides or fragments thereof by recombinant techniques.

As used herein, a “vector” is any construct capable of delivering one or more polynucleotide(s) of interest to a host cell when the vector is introduced to the host cell. An “expression vector” is capable of delivering and expressing the one or more polynucleotide(s) of interest as an encoded polypeptide in a host cell into which the expression vector has been introduced. Thus, in an expression vector, the polynucleotide of interest is positioned for expression in the vector by being operably linked with regulatory elements such as a promoter, enhancer, and/or a poly-A tail, either within the vector or in the genome of the host cell at or near or flanking the integration site of the polynucleotide of interest such that the polynucleotide of interest will be translated in the host cell introduced with the expression vector. In some embodiments, the vector described herein can have a schematic structure shown in FIGS. 3A-3B, 5A, and FIGS. 8A-8B.

A vector can be introduced into the host cell by methods known in the art, e.g., electroporation, chemical transfection (e.g., DEAE-dextran), transformation, transfection, and infection and/or transduction (e.g., with recombinant virus). Thus, non-limiting examples of vectors include viral vectors (which can be used to generate recombinant virus), naked DNA or RNA, plasmids, cosmids, phage vectors, and DNA or RNA expression vectors associated with cationic condensing agents.

The present disclosure provides a recombinant vector comprising a nucleic acid construct suitable for genetically modifying a cell, which can be used for treatment of pathological disease or condition.

Any vector or vector type can be used to deliver genetic material to the cell. These vectors include but are not limited to plasmid vectors, viral vectors, bacterial artificial chromosomes (BACs), yeast artificial chromosomes (YACs), and human artificial chromosomes (HACs). Viral vectors can include but are not limited to recombinant retroviral vectors, recombinant lentiviral vectors, recombinant adenoviral vectors, foamy virus vectors, recombinant adeno-associated viral (AAV) vectors, hybrid vectors, and plasmid transposons (e.g., sleeping beauty transposon system, and PiggyBac transposon system) or integrase based vector systems. Other vectors that are known in the art can also be used in connection with the methods described herein.

In some embodiments, the vector is a viral vector. The viral vector can be grown in a culture medium specific for viral vector manufacturing. Any suitable growth media and/or supplements for growing viral vectors can be used in accordance with the embodiments described herein.

In some embodiments, the vector used is a recombinant retroviral vector. A retroviral vector is capable of directing the expression of a nucleic acid molecule of interest. A retrovirus is present in the RNA form in its viral capsule and forms a double-stranded DNA intermediate when it replicates in the host cell. Similarly, retroviral vectors are present in both RNA and double-stranded DNA forms. The retroviral vector also includes the DNA form which contains a recombinant DNA fragment and the RNA form containing a recombinant RNA fragment. The vectors can include at least one transcriptional promoter/enhancer, or other elements which control gene expression. Such vectors can also include a packaging signal, long terminal repeats (LTRs) or portion thereof, and positive and negative strand primer binding sites appropriate to the retrovirus used. Long terminal repeats (LTRs) are identical sequences of DNA that repeat many times (e.g., hundreds or thousands of times) found at either end of retrotransposons or proviral DNA formed by reverse transcription of retroviral RNA. They are used by viruses to insert their genetic material into the host genomes. Optionally, the vectors can also include a signal which directs polyadenylation, selectable markers such as Ampicillin resistance, Neomycin resistance, TK, hygromycin resistance, phleomycin resistance histidinol resistance, or DHFR, as well as one or more restriction sites and a translation termination sequence. For example, such vectors can include a 5′LTR, a leading sequence, a tRNA binding site, a packaging signal, an origin of second strand DNA synthesis, and a 3′ LTR or a portion thereof. Additionally, retroviral vector used herein can also refers to the recombinant vectors created by removal of the retroviral gag, pol, and env genes and replaced with the gene of interest.

In some embodiments, the vector can include an additional nucleic acid encoding an inhibitory protein (e.g., a checkpoint inhibitor). In various embodiments, the cell expresses the genetically engineered antigen receptor and the inhibitory protein. In various embodiments, the inhibitory protein is constitutively expressed.

In some embodiments, the vector or construct can contain a single promoter that drives the expression of one or more nucleic acid molecules. In some embodiments, such promoters can be multicistronic (bicistronic or tricistronic). For example, in some embodiments, transcription units can be engineered as a bicistronic unit containing an IRES (internal ribosome entry site), which allows coexpression of gene products (e.g., encoding an alpha chain and/or beta chain of a TCR) by a message from a single promoter. Alternatively, in some cases, a single promoter may direct expression of an RNA that contains, in a single open reading frame (ORF), two or three genes separated from one another by sequences encoding a self-cleavage peptide (e.g., P2A or T2A) or a protease recognition site (e.g., furin). The ORF thus encodes a single polyprotein, which, either during (in the case of 2A e.g., T2A) or after translation, is cleaved into the individual proteins. In some cases, the peptide, such as T2A, can cause the ribosome to skip (ribosome skipping) synthesis of a peptide bond at the C-terminus of a 2A element, leading to separation between the end of the 2A sequence and the next peptide downstream.

Various cell lines can be used in connection with the vectors as described herein. Exemplary eukaryotic cells that may be used to express polypeptides include, but are not limited to, COS cells, including COS 7 cells; 293 cells, including 293-6E cells; CHO cells, including CHO-S, DG44. Lec13 CHO cells, and FUT8 CHO cells; PER.C6® cells; and NSO cells. In some embodiments, a particular eukaryotic host cell is selected based on its ability to make desired post-translational modifications to the binding molecule. For example, in some embodiments, CHO cells produce polypeptides that have a higher level of sialylation than the same polypeptide produced in 293 cells.

The term “Linker” (L) or “linker peptide” as used herein refer to an oligo- or polypeptide region from about 1 to 100 amino acids in length, which links together any of the domains/regions. Linkers can be composed of flexible residues like glycine and serine so that the adjacent protein domains are free to move relative to one another. Longer linkers can be used when it is desirable to ensure that two adjacent domains do not sterically interfere with one another. Linkers can be cleavable or non-cleavable. Examples of cleavable linkers include 2A linkers (for example P2A, T2A), 2A-like linkers or functional equivalents thereof and combinations thereof. In some embodiments, the linkers include the picornaviral 2A-like linker, CHYSEL sequences of porcine teschovirus (P2A), Thosea asigna virus (T2A) or combinations, variants and functional equivalents thereof. Other linkers will be apparent to those of skill in the art and can be used in the methods described herein.

The successful construction of a recombinant fusion protein often requires two elements: the component proteins and the linkers. The choice of the component proteins is based on the desired functions of the fusion protein product and, in most cases, is relatively straightforward. On the other hand, the selection of a suitable linker to join the protein domains together can be complicated and is often neglected in the design of fusion proteins. Direct fusion of functional domains without a linker may lead to many undesirable outcomes, including misfolding of the fusion proteins, low yield in protein production, or impaired bioactivity. Therefore, the selection or rational design of a linker to join fusion protein domains is an important, yet underexplored, area in recombinant fusion protein technology. Details of linker design, especially the selection between flexible linkers and rigid linkers, can be found, e.g., in Chen, X. et al. “Fusion protein linkers: property, design and functionality.” Advanced Drug Delivery Reviews 65.10 (2013): 1357-1369, which is incorporated herein by reference in its entirety.

In some embodiments, the fusion proteins or fusion polypeptides described herein includes a linker that connects a first moiety and a second moiety. In some embodiments, the linker is a flexible linker, e.g., a linker with an amino acid sequence that is at least 70%, 75%, 80%, 85%, 90%, 95%, 96%, 97%, 98%, 99%, or 100% identical to SEQ ID NO: 4, 11, or 28. In some embodiments, the linker includes an amino acid sequence that is at least 80%, 85%, 90%, 95%, or 100% identical to one or more (e.g., 1, 2, 3, 4, 5, 6, 7, or 8) repeats of GGGGS (SEQ ID NO: 29) or GGGS (SEQ ID NO: 30). In some embodiments, at least 50%, 60%, 70%, 80%, or 90% of the amino acid residues in the flexible linker are glycine residues.

The present disclosure also provides a nucleic acid sequence comprising a nucleotide sequence encoding any of the chimeric polypeptides (or the fusion polypeptide), antibodies or antigen-binding fragments thereof, and/or peptibodies described herein. “Nucleic acid” as used herein can include “polynucleotide,” “oligonucleotide,” and “nucleic acid molecule,” and generally means a polymer of DNA or RNA, which can be single-stranded or double-stranded, synthesized or obtained from natural sources, which can contain natural, non-natural or altered nucleotides. Furthermore, the nucleic acid comprises complementary DNA (cDNA). It is generally preferred that the nucleic acid does not comprise any insertions, deletions, inversions, and/or substitutions. However, it can be suitable in some instances, as discussed herein, for the nucleic acid to comprise one or more insertions, deletions, inversions, and/or substitutions.

The nucleic acids as described herein can be constructed based on chemical synthesis and/or enzymatic ligation reactions using procedures known in the art. For example, a nucleic acid can be chemically synthesized using naturally occurring nucleotides or variously modified nucleotides. In some of any such embodiments, the nucleotide sequence is codon-optimized.

The present disclosure also provides the nucleic acids comprising a nucleotide sequence complementary to the nucleotide sequence of any of the nucleic acids described herein or a nucleotide sequence which hybridizes under stringent conditions to the nucleotide sequence of any of the nucleic acids described herein. In some embodiments, the nucleic acid is synthetic. In some embodiments, the nucleic acid is cDNA.

In some of any such embodiments, the chimeric polypeptides (or the fusion polypeptide), antibodies or antigen-binding fragments thereof, and/or peptibodies described herein are encoded by a nucleotide sequence that has been codon-optimized.

The disclosure also provides a nucleic acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any nucleotide sequence as described herein, and an amino acid sequence that is at least 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 91%, 92%, 93%, 94%, 95%, 96%, 97%, 98%, 99% identical to any amino acid sequence as described herein. In some embodiments, the disclosure relates to nucleotide sequences encoding any peptides that are described herein, or any amino acid sequences that are encoded by any nucleotide sequences as described herein.

In some embodiments, the nucleic acid sequence is at least or about 10, 20, 30, 40, 50, 60, 70, 80, 90, 100, 110, 120, 130, 150, 200, 250, 300, 350, 400, 500, or 600 nucleotides. In some embodiments, the amino acid sequence is at least or about 5, 6, 7, 8, 9, 10, 20, 30, 40, 50, 60, 70, 80, 90, 100, 110, 120, 130, 140, 150, 160, 170, 180, 190, or 200 amino acid residues. In some embodiments, the nucleic acid sequence is less than 10, 20, 30, 40, 50, 60, 70, 80, 90, 100, 110, 120, 130, 150, 200, 250, 300, 350, 400, 500, or 600 nucleotides. In some embodiments, the amino acid sequence is less than 5, 6, 7, 8, 9, 10, 20, 30, 40, 50, 60, 70, 80, 90, 100, 110, 120, 130, 140, 150, 160, 170, 180, 190, or 200 amino acid residues.

To determine the percent identity of two amino acid sequences, or of two nucleic acid sequences, the sequences are aligned for optimal comparison purposes (e.g., gaps can be introduced in one or both of a first and a second amino acid or nucleic acid sequence for optimal alignment and non-homologous sequences can be disregarded for comparison purposes). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

### Method for Preparation of Engineered Cells

The present disclosure provides a method or process for manufacturing and using the engineered cells for treatment of pathological diseases or conditions.

The cells for introduction of the molecule, e.g., the chimeric polypeptides (or the fusion polypeptide), antibodies or antigen-binding fragments thereof, and/or peptibodies described herein, can be isolated from a sample, such as a biological sample, e.g., one obtained from or derived from a subject. In some embodiments, the subject from which the cell is isolated is one having the disease or condition or in need of a cell therapy or to which cell therapy will be administered. The subject in some embodiments is a human in need of a particular therapeutic intervention, such as the adoptive cell therapy for which cells are being isolated, processed, and/or engineered.

Accordingly, the cells in some embodiments are primary cells, e.g., primary human cells. In some embodiments, the cells are cord blood-derived NK cells. The samples include tissue, fluid, and other samples taken directly from the subject, as well as samples resulting from one or more processing steps, such as separation, centrifugation, genetic engineering (e.g., transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines, e.g., NK cell lines. The cells in some embodiments are obtained from a xenogeneic source, for example, from mouse, rat, or non-human primate.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge. In some aspects, a washing step is accomplished by tangential flow filtration (TFF). In some embodiments, the cells are resuspended in a variety of biocompatible buffers after washing, such as, for example, Ca²⁺/Mg²⁺ free PBS. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In some embodiments, the method comprises one or more steps of: e.g., isolating the NK cells from a patient's cord blood; transducing the population of NK cells with a viral vector including the nucleic acid construct encoding the chimeric polypeptides (or the fusion polypeptide), antibodies or antigen-binding fragments thereof, and/or peptibodies described herein; expanding the transduced cells in vitro; and/or infusing the expanded cells into the patient, where the engineered NK cells will seek and destroy tumor cells expressing a target antigen or receptor. In some embodiments, the method further comprises: transfection of NK cells with the viral vector containing the nucleic acid construct.

In some embodiments, the methods involve introducing any vectors described herein into a cell in vitro or ex vivo. In some embodiments, the vector is a viral vector and the introducing is carried out by transduction. In some embodiments, the methods further involve introducing into the cell one or more agents, wherein each of the one or more agents is independently capable of inducing a genetic disruption. In some embodiments, the one or more agent is an inhibitory nucleic acid (e.g., siRNA). In some embodiments, the one or more agent is a fusion protein comprising a DNA-targeting protein and a nuclease or an RNA-guided nuclease (e.g., a clustered regularly interspaced short palindromic nucleic acid (CRISPR)-associated nuclease).

The transfection of immune cells (e.g., NK cells) may be achieved by using any standard method such as calcium phosphate, electroporation, liposomal mediated transfer, microinjection, biolisticparticle delivery system, or any other known methods by skilled artisan. In some embodiments, transfection of immune cells is performed using the calcium phosphate method.

Methods of preparing engineered cells and administering these engineered cells to a subject are known in the art, and are described e.g., in Schmidt, P., et al. “Engineering NK cells for CAR therapy-recent advances in gene transfer methodology.” Frontiers in Immunology 11 (2021): 611163; and Savan, R., et al. “Lentiviral gene transduction in human and mouse NK cell lines.” Natural Killer Cell Protocols: Cellular and Molecular Methods. Totowa, NJ: Humana Press, 2009. 209-221; both of which are incorporated by reference in their entirety.

### Methods of Treatment

The methods disclosed herein can be used for various therapeutic purposes. In one aspect, the disclosure provides methods for treating a cancer in a subject, methods of reducing the rate of the increase of volume of a tumor in a subject over time, methods of reducing the risk of developing a metastasis, or methods of reducing the risk of developing an additional metastasis in a subject. In some embodiments, the treatment can halt, slow, retard, or inhibit progression of a cancer. In some embodiments, the treatment can result in the reduction of in the number, severity, and/or duration of one or more symptoms of the cancer in a subject.

In one aspect, the disclosure features methods that include administering a therapeutically effective amount (e.g., number) of engineered cells (e.g., any of the engineered immune cells described herein) to a subject in need thereof (e.g., a subject having, or identified or diagnosed as having, a cancer).

The invention provides methods for treating cancer using the engineered immune cells (e.g., NK cells) described herein and/or a pharmaceutical composition described herein. The methods may be used to treat a variety of cancers, including a solid tumor, a lymphoma, and a leukemia. The type of cancer to be treated is desirably matched with the type of cancer cell to which the antibody or antigen-binding fragment thereof or peptibody (e.g., any of the antibodies or antigen-binding fragment thereof, or any of the peptibodies described herein) binds. For example, treatment of a cancer expressing PD-L1 and/or IL13Rα2, is desirably treated using the engineered immune cells (e.g., NK cells) described herein that express and/or secret an anti-PD-L1 antibody or antigen-binding fragment thereof and/or anti-IL13Rα2 peptibody. Additional aspects and embodiments of the therapeutic methods are described below.

Accordingly, one aspect of the disclosure provides a method of treating cancer in a patient, wherein the method comprises administering to a patient in need thereof a therapeutically effective number of the engineered immune cells (e.g., NK cells) described herein to treat the cancer. Exemplary cancers for treatment include a solid tumor, leukemia, and lymphoma.

The therapeutic method can be characterized according to the cancer to be treated. For example, in certain embodiments, the cancer is a solid tumor. In certain other embodiments, the cancer is brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, or uterine cancer. In yet other embodiments, the cancer is a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, bilary tract cancer, thyroid cancer, acral lentiginous melanoma, actinic keratoses, acute lymphocytic leukemia, acute myeloid leukemia, adenoid cycstic carcinoma, adenomas, adenosarcoma, adenosquamous carcinoma, anal canal cancer, anal cancer, anorectum cancer, astrocytic tumor, bartholin gland carcinoma, basal cell carcinoma, biliary cancer, bone cancer, bone marrow cancer, bronchial cancer, bronchial gland carcinoma, carcinoid, cholangiocarcinoma, chondosarcoma, choriod plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangiblastomas, hemangioendothelioma, hemangiomas, hepatic adenoma, hepatic adenomatosis, hepatobiliary cancer, hepatocellular carcinoma, Hodgkin's disease, ileum cancer, insulinoma, intaepithelial neoplasia, interepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor.

In certain other embodiments, the cancer is non-Hodgkin's lymphoma, such as a B-cell lymphoma or a T-cell lymphoma. In certain embodiments, the non-Hodgkin's lymphoma is a B-cell lymphoma, such as a diffuse large B-cell lymphoma, primary mediastinal B-cell lymphoma, follicular lymphoma, small lymphocytic lymphoma, mantle cell lymphoma, marginal zone B-cell lymphoma, extranodal marginal zone B-cell lymphoma, nodal marginal zone B-cell lymphoma, splenic marginal zone B-cell lymphoma, Burkitt lymphoma, lymphoplasmacytic lymphoma, hairy cell leukemia, or primary central nervous system (CNS) lymphoma. In certain other embodiments, the non-Hodgkin's lymphoma is a T-cell lymphoma, such as a precursor T-lymphoblastic lymphoma, peripheral T-cell lymphoma, cutaneous T-cell lymphoma, angioimmunoblastic T-cell lymphoma, extranodal natural killer/T-cell lymphoma, enteropathy type T-cell lymphoma, subcutaneous panniculitis-like T-cell lymphoma, anaplastic large cell lymphoma, or peripheral T-cell lymphoma.

The cancer to be treated can be characterized according to the presence of a particular antigen expressed on the surface of the cancer cell. In certain embodiments, the cancer cell expresses one or more of the following: PD-L1, BCMA, Caudin18.2, CCR4, CD10, CD123, CD147, CD171, CD19, CD20, CD22, CD276, CD319, CD33, CD38, CD70, CLL-1, DLL3, EGFR, EGFRVIII, EpCAM, FLT3, FRα, GD2, GPC3, HER2, HGFR, IL13Ralpha2, Mesothelin, MG7, MUC1, MUC16, Nectin4, PSCA, ROR1, ROR2, TACI, TRBC1, TSLPR, and VEGFR. In some embodiments, the cancer cell expresses one or more of the following: IL13Rα2, APN/CD13, APP, PD-L1, CD44, P32/gC1qR, E-cadherin, N-cadherin, CD21, EGFR, Epha2, EphB4, HER2, FGFR1, FGFR2, FGFR3, FGFR4, VEGFR1, VEGFR3, PSMA, GPC3, IL-10RA, IL-11Rα, IL-6Rα, GP130, VEGFR2, MUC18, Met, MMP9, Thomsen-Friedenreich carbohydrate antigen, NRP-1, PDGFRβ, CD133, PTPRJ, HSPG, E-selectin, Tie2, VPAC1, ActRIIB, CD163, CXCR4, Ephrin A4, Ephrin B1, Ephrin B2, Ephrin B3, Gonadotrophin releasing hormone receptor, G Protein-coupled receptor 55, Bombesin receptor 2, IL4 receptor, Low-density lipoprotein receptor, Leptin receptor, LRP1, Melanocortin 1 receptor, Melanocortin 4 receptor, CD206, Urokinase plasminogen activator receptor, Neurokinin-1 receptor, VPAC2, ITGB1, CD27, ITGB5, ITGA1, CD27, LRP1, ACVR2B, COL13A1, NOTCH3, EGFR, VEGFR2, VEGFR3, PDGFR, HER2, ErbB3, ErbB4, RET, and FGFR102. In some embodiments, the cancer cell expresses one or more of the antigens in Table 2 and/or one or more of the receptors in Table 4.

In some embodiments, the compositions and methods disclosed herein can be used for treatment of patients at risk for a cancer. Patients with cancer can be identified with various methods known in the art.

As used herein, by an “effective amount” is meant an amount or dosage sufficient to effect beneficial or desired results including halting, slowing, retarding, or inhibiting progression of a disease, e.g., a cancer. An effective amount will vary depending upon, e.g., an age and a body weight of a subject to which the therapeutic agent and/or therapeutic compositions is to be administered, a severity of symptoms and a route of administration, and thus administration can be determined on an individual basis.

An effective amount can be administered in one or more administrations. By way of example, an effective amount of a composition is an amount sufficient to ameliorate, stop, stabilize, reverse, inhibit, slow and/or delay progression of a cancer in a patient or is an amount sufficient to ameliorate, stop, stabilize, reverse, slow and/or delay proliferation of a cell (e.g., a biopsied cell, any of the cancer cells described herein, or cell line (e.g., a cancer cell line)) in vitro. As is understood in the art, an effective may vary, depending on, inter alia, patient history as well as other factors such as the type (and/or dosage) of compositions used.

Effective amounts and schedules for administrations may be determined empirically, and making such determinations is within the skill in the art. Those skilled in the art will understand that the dosage that must be administered will vary depending on, for example, the mammal that will receive the treatment, the route of administration, the particular type of therapeutic agents and other drugs being administered to the mammal. Guidance in selecting appropriate doses can be found in the literature. In addition, a treatment does not necessarily result in the 100% or complete treatment or prevention of a disease or a condition. There are multiple treatment/prevention methods available with a varying degree of therapeutic effect which one of ordinary skill in the art recognizes as a potentially advantageous therapeutic mean.

In any of the methods described herein, the engineered cells and, and/or at least one additional therapeutic agent can be administered to the subject at least once a week (e.g., once a week, twice a week, three times a week, four times a week, once a day, twice a day, or three times a day). In some embodiments, at least two different engineered cells (e.g., cells express different binding molecules) are administered in the same composition (e.g., a liquid composition). In some embodiments, engineered cells and at least one additional therapeutic agent are administered in the same composition (e.g., a liquid composition). In some embodiments, engineered cells and the at least one additional therapeutic agent are administered in two different compositions. In some embodiments, the at least one additional therapeutic agent is administered as a pill, tablet, or capsule. In some embodiments, the at least one additional therapeutic agent is administered in a sustained-release oral formulation.

Exemplary therapeutic agents that may be used as part of a combination therapy in treating cancer, include, for example, radiation, mitomycin, tretinoin, ribomustin, gemcitabine, vincristine, etoposide, cladribine, mitobronitol, methotrexate, doxorubicin, carboquone, pentostatin, nitracrine, zinostatin, cetrorelix, letrozole, raltitrexed, daunorubicin, fadrozole, fotemustine, thymalfasin, sobuzoxane, nedaplatin, cytarabine, bicalutamide, vinorelbine, vesnarinone, aminoglutethimide, amsacrine, proglumide, elliptinium acetate, ketanserin, doxifluridine, etretinate, isotretinoin, streptozocin, nimustine, vindesine, flutamide, drogenil, butocin, carmofur, razoxane, sizofilan, carboplatin, mitolactol, tegafur, ifosfamide, prednimustine, picibanil, levamisole, teniposide, improsulfan, enocitabine, lisuride, oxymetholone, tamoxifen, progesterone, mepitiostane, epitiostanol, formestane, interferon-alpha, interferon-2 alpha, interferon-beta, interferon-gamma, colony stimulating factor-1, colony stimulating factor-2, denileukin diftitox, interleukin-2, luteinizing hormone releasing factor and variations of the aforementioned agents that may exhibit differential binding to its cognate receptor, and increased or decreased serum half-life.

The number of the engineered immune cells (e.g., NK cells) described herein and additional therapeutic agent and the relative timing of administration may be selected in order to achieve a desired combined therapeutic effect. For example, when administering a combination therapy to a patient in need of such administration, the therapeutic agents in the combination, or a pharmaceutical composition or compositions comprising the therapeutic agents, may be administered in any order such as, for example, sequentially, concurrently, together, simultaneously and the like. Further, for example, a protein described herein may be administered during a time when the additional therapeutic agent(s) exerts its prophylactic or therapeutic effect, or vice versa.

In some embodiments, one or more additional therapeutic agents can be administered to the subject. The additional therapeutic agent can be a checkpoint inhibitor (CPI). In some embodiments, the checkpoint inhibitor is an inhibitory protein, e.g., an antibody or antigen binding fragment thereof. The checkpoint inhibitor can inhibit or block one or more immune checkpoints, including e.g., PD-1, PD-L1, PD-L2, 2B4 (CD244), 4-1BB, A2aR, B7.1, B7.2, B7-H2, B7-H3, B7-H4, B7-H6, BTLA, butyrophilins, CD160, CD48, CTLA4, GITR, gp49B, HHLA2, HVEM, ICOS, ILT-2, ILT-4, KIR family receptors, LAG-3, OX-40, PIR-B, SIRPalpha (CD47), TFM-4, TIGIT, TIM-1, TIM-3, TIM-4, VISTA and combinations thereof. In some embodiments, the inhibitory protein blocks PD-1 or PD-L1. In various embodiments, the inhibitory protein comprises an anti-PD-1 scFv. The inhibitory protein is capable of leading to reduced expression of PD-1 or PD-L1 and/or inhibiting upregulation of PD-1 or PD-L1 in T cells in the population and/or physically obstructing the formation of the PD-1/PD-L1 complex and subsequent signal transduction. In some embodiments, the inhibitory protein blocks PD-1. In some embodiments, the additional therapeutic agent is an anti-OX40 antibody, an anti-PD-L1 antibody, an anti-PD-L2 antibody, an anti-LAG-3 antibody, an anti-TIGIT antibody, an anti-BTLA antibody, an anti-CTLA-4 antibody, or an anti-GITR antibody. In some embodiments, the additional therapeutic agent is an anti-CTLA4 antibody (e.g., ipilimumab), an anti-CD20 antibody (e.g., rituximab), an anti-EGFR antibody (e.g., cetuximab), an anti-CD319 antibody (e.g., elotuzumab), or an anti-PD1 antibody (e.g., nivolumab).

Yet other agents that may be used as part of a combination therapy in treating cancer are monoclonal antibody agents that target non-checkpoint targets (e.g., herceptin) and non-cytotoxic agents (e.g., tyrosine-kinase inhibitors).

In one some embodiments, the additional therapeutic agent can comprise one or more inhibitors selected from the group consisting of an inhibitor of B-Raf, an EGFR inhibitor, an inhibitor of a MEK, an inhibitor of ERK, an inhibitor of K-Ras, an inhibitor of c-Met, an inhibitor of anaplastic lymphoma kinase (ALK), an inhibitor of a phosphatidylinositol 3-kinase (PI3K), an inhibitor of an Akt, an inhibitor of mTOR, a dual PI3K/mTOR inhibitor, an inhibitor of Bruton's tyrosine kinase (BTK), and an inhibitor of Isocitrate dehydrogenase 1 (IDH1) and/or Isocitrate dehydrogenase 2 (IDH2). In some embodiments, the additional therapeutic agent is an inhibitor of indoleamine 2,3-dioxygenase-1) (IDO1) (e.g., epacadostat). In some embodiments, the additional therapeutic agent can comprise one or more inhibitors selected from the group consisting of an inhibitor of HER3, an inhibitor of LSD1, an inhibitor of MDM2, an inhibitor of BCL2, an inhibitor of CHK1, an inhibitor of activated hedgehog signaling pathway, and an agent that selectively degrades the estrogen receptor. Yet other categories of anti-cancer agents include, for example: (i) an inhibitor selected from an ALK Inhibitor, an ATR Inhibitor, an A2A Antagonist, a Base Excision Repair Inhibitor, a Ber-Abl Tyrosine Kinase Inhibitor, a Bruton's Tyrosine Kinase Inhibitor, a CDC7 Inhibitor, a CHK1 Inhibitor, a Cyclin-Dependent Kinase Inhibitor, a DNA-PK Inhibitor, an Inhibitor of both DNA-PK and mTOR, a DNMT1 Inhibitor, a DNMT1 Inhibitor plus 2-chloro-deoxyadenosine, an HDAC Inhibitor, a Hedgehog Signaling Pathway Inhibitor, an IDO Inhibitor, a JAK Inhibitor, a mTOR Inhibitor, a MEK Inhibitor, a MELK Inhibitor, a MTH1 Inhibitor, a PARP Inhibitor, a Phosphoinositide 3-Kinase Inhibitor, an Inhibitor of both PARP1 and DHODH, a Proteasome Inhibitor, a Topoisomerase-II Inhibitor, a Tyrosine Kinase Inhibitor, a VEGFR Inhibitor, and a WEE1 Inhibitor; (ii) an agonist of OX40, CD137, CD40, GITR, CD27, HVEM, TNFRSF25, or ICOS; and (iii) a cytokine selected from IL-12, IL-15, GM-CSF, and G-CSF.

In some embodiments, the additional therapeutic agent can comprise one or more therapeutic agents selected from the group consisting of Trabectedin, nab-paclitaxel, Trebananib, Pazopanib, Cediranib, Palbociclib, everolimus, fluoropyrimidine, IFL, regorafenib, Reolysin, Alimta, Zykadia, Sutent, temsirolimus, axitinib, everolimus, sorafenib, Votrient, Pazopanib, IMA-901, AGS-003, cabozantinib, Vinflunine, an Hsp90 inhibitor, Ad-GM-CSF, Temazolomide, IL-2, IFNa, vinblastine, Thalomid, dacarbazine, cyclophosphamide, lenalidomide, azacytidine, lenalidomide, bortezomid, amrubicine, carfilzomib, pralatrexate, and enzastaurin.

In some embodiments, the additional therapeutic agent can comprise one or more therapeutic agents selected from the group consisting of an adjuvant, a TLR agonist, tumor necrosis factor (TNF) alpha, IL-1, HMGB1, an IL-10 antagonist, an IL-4 antagonist, an IL-13 antagonist, an IL-17 antagonist, an HVEM antagonist, an ICOS agonist, a treatment targeting CX3CL1, a treatment targeting CXCL9, a treatment targeting CXCL10, a treatment targeting CCL5, an LFA-1 agonist, an ICAM1 agonist, and a Selectin agonist.

In some embodiments, carboplatin, nab-paclitaxel, paclitaxel, cisplatin, pemetrexed, gemcitabine, FOLFOX, or FOLFIRI are administered to the subject. In some embodiments, the additional therapeutic agent is selected from asparaginase, busulfan, carboplatin, cisplatin, daunorubicin, doxorubicin, fluorouracil, gemcitabine, hydroxyurea, methotrexate, paclitaxel, rituximab, vinblastine, vincristine and/or combinations thereof.

### Compositions and Formulations

The present disclosure provides compositions (including pharmaceutical and therapeutic compositions) containing the engineered cells and populations thereof, produced by the methods disclosed herein. Also provided are methods, e.g., therapeutic methods for administrating the engineered immune cells (e.g., NK cells) and compositions thereof to subjects, e.g., patients.

Compositions including the engineered immune cells for administration, including pharmaceutical compositions and formulations, such as unit dose form compositions including the number of cells for administration in a given dose or fraction thereof are provided. The pharmaceutical compositions and formulations can include one or more optional pharmaceutically acceptable carrier or excipient. In some embodiments, the composition includes at least one additional therapeutic agent.

A pharmaceutically acceptable carrier refers to an ingredient in a pharmaceutical composition, other than an active ingredient. The pharmaceutically acceptable carrier does not interfere with the active ingredient and is nontoxic to a subject. A pharmaceutically acceptable carrier can include, but is not limited to, a buffer, excipient, stabilizer, or preservative. The pharmaceutical formulation refers to process in which different substances and/or agents are combined to produce a final medicinal product. The formulation studies involve developing a preparation of drug acceptable for patient. Additionally, a preparation which is in such form as to permit the biological activity of an active ingredient contained therein to be effective, and which contains no additional components which are unacceptably toxic to a subject to which the formulation would be administered.

In some embodiments, the choice of carrier is determined in part by the particular cell (e.g., T cell or NK cell) and/or by the method of administration. A variety of suitable formulations are available. For example, the pharmaceutical composition can contain preservatives. Suitable preservatives can include, for example, methylparaben, propylparaben, sodium benzoate, and benzalkonium chloride. In some embodiments, a mixture of two or more preservatives is used. The preservative or mixtures thereof are typically present in an amount of about 0.0001% to about 2% by weight of the total composition. Carriers are described, e.g., by Remington's Pharmaceutical Sciences 16th edition, Osol, A. Ed. (1980). Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

Suitable buffering agents include, for example, citric acid, sodium citrate, phosphoric acid, potassium phosphate, and various other acids and salts. In some embodiments, a mixture of two or more buffering agents is used. The buffering agent or mixtures thereof are typically present in an amount of about 0.001% to about 4% by weight of the total composition. Methods for preparing administrable pharmaceutical compositions are known. Exemplary methods are described in more detail in, for example, Remington: The Science and Practice of Pharmacy, Lippincott Williams & Wilkins; 21st ed. (May 1, 2005).

The formulations can include aqueous solutions. The formulation or composition can also contain more than one active ingredient useful for a particular indication, disease, or condition being treated with the engineered cells, preferably those with activities complementary to the cells, where the respective activities do not adversely affect one another. Such active ingredients are suitably present in combination in amounts that are effective for the purpose intended. Thus, in some embodiments, the pharmaceutical composition can further include other pharmaceutically active agents or drugs, such as checkpoint inhibitors, fusion proteins, chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, and/or vincristine.

The pharmaceutical composition in some embodiments contains the cells in amounts effective to treat or prevent the disease or condition, such as a therapeutically effective or prophylactically effective amount. Therapeutic or prophylactic efficacy in some embodiments is monitored by periodic assessment of treated subjects. The desired dosage can be delivered by a single bolus administration of the cells, by multiple bolus administrations of the cells, or by continuous infusion administration of the cells.

The cells and compositions can be administered using standard administration techniques, formulations, and/or devices. Administration of the cells can be autologous or heterologous. For example, immunoresponsive T cells or progenitors can be obtained from one subject, and administered to the same subject or a different, compatible subject after genetically modifying them in accordance with various embodiments described herein. Peripheral blood derived immunoresponsive T cells or their progeny (e.g., in vivo, ex vivo or in vitro derived) can be administered via localized injection, including catheter administration, systemic injection, localized injection, intravenous injection, or parenteral administration. Usually, when administering a therapeutic composition (e.g., a pharmaceutical composition containing a genetically modified immunoresponsive cell), it is generally formulated in a unit dosage injectable form (solution, suspension, emulsion).

Formulations disclosed herein include those for oral, intravenous, intraperitoneal, subcutaneous, pulmonary, transdermal, intramuscular, intranasal, buccal, sublingual, or suppository administration. In some embodiments, the cell populations are administered parenterally. The term “parenteral,” as used herein, includes intravenous, intramuscular, subcutaneous, rectal, vaginal, and intraperitoneal administration. In some embodiments, the cells are administered to the subject using peripheral systemic delivery by intravenous, intraperitoneal, or subcutaneous injection.

The compositions in some embodiments are provided as sterile liquid preparations, e.g., isotonic aqueous solutions, suspensions, emulsions, dispersions, or viscous compositions, which can in some aspects be buffered to a selected pH. Liquid preparations are normally easier to prepare than gels, other viscous compositions, and solid compositions. Additionally, liquid compositions are somewhat more convenient to administer, especially by injection. Viscous compositions, on the other hand, can be formulated within the appropriate viscosity range to provide longer contact periods with specific tissues. Liquid or viscous compositions can comprise carriers, which can be a solvent or dispersing medium containing, for example, water, saline, phosphate buffered saline, polyol (for example, glycerol, propylene glycol, liquid polyethylene glycol) and suitable mixtures thereof.

Sterile injectable solutions can be prepared by incorporating the cells in a solvent, such as in admixture with a suitable carrier, diluent, or excipient such as sterile water, physiological saline, glucose, dextrose, or the like. The compositions can contain auxiliary substances such as wetting, dispersing, or emulsifying agents (e.g., methylcellulose), pH buffering agents, gelling or viscosity enhancing additives, preservatives, flavoring agents, and/or colors, depending upon the route of administration and the preparation desired. Standard texts can in some aspects be consulted to prepare suitable preparations.

Various additives which enhance the stability and sterility of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, and sorbic acid. Prolonged absorption of the injectable pharmaceutical form can be brought about by the use of agents delaying absorption, for example, aluminum monostearate and gelatin.

The formulations to be used for in vivo administration are generally sterile. Sterility can be readily accomplished, e.g., by filtration through sterile filtrationmembranes.

The compositions or pharmaceutical compositions as described herein can be included in a container, pack, or dispenser together with instructions for administration.

### Methods of Administration

Provided are also methods of administering the cells, populations, and compositions, and uses of such cells, populations, and compositions to treat or prevent diseases, conditions, and disorders, including cancers. In some embodiments, the methods described herein can reduce the risk of the developing diseases, conditions, and disorders as described herein.

In some embodiments, the cells, populations, and compositions, described herein are administered to a subject or patient having a particular disease or condition to be treated, e.g., via adoptive cell therapy, such as adoptive NK cell therapy. In some embodiments, cells and compositions prepared by the provided methods, such as engineered compositions and end-of-production compositions following incubation and/or other processing steps, are administered to a subject, such as a subject having or at risk for the disease or condition. In some aspects, the methods thereby treat, e.g., ameliorate one or more symptom of, the disease or condition, such as by lessening tumor burden in cancer expressing an antigen recognized by the engineered T cells.

Methods for administration of cells for adoptive cell therapy are known and can be used in connection with the provided methods and compositions. For example, adoptive cell therapy methods are described, e.g., in U.S. 2003/0170238; U.S. Pat. No. 4,690,915; Rosenberg, “Cell transfer immunotherapy for metastatic solid cancer-what clinicians need to know.” Nature reviews Clinical oncology 8.10 (2011): 577; Themeli et al. “Generation of tumor-targeted human T lymphocytes from induced pluripotent stem cells for cancer therapy.” Nature biotechnology 31.10 (2013): 928; Tsukahara et al. “CD19 target-engineered T-cells accumulate at tumor lesions in human B-cell lymphoma xenograft mouse models.” Biochemical and biophysical research communications 438.1 (2013): 84-89; Davila et al. “CD19 CAR-targeted T cells induce long-term remission and B Cell Aplasia in an immunocompetent mouse model of B cell acute lymphoblastic leukemia.” PloS one 8.4 (2013); each of which is incorporated herein by reference in its entirety.

In some embodiments, the cell therapy, e.g., adoptive cell therapy, is carried out by autologous transfer, in which the T cells are isolated and/or otherwise prepared from the subject who is to receive the cell therapy, or from a sample derived from such a subject. Thus, in some aspects, the cells are derived from a subject, e.g., patient, in need of a treatment and the cells, following isolation and processing are administered to the same subject.

In some embodiments, the cell therapy, e.g., adoptive cell therapy, is carried out by allogeneic transfer, in which the T cells are isolated and/or otherwise prepared from a subject other than a subject who is to receive or who ultimately receives the cell therapy, e.g., a first subject. In such embodiments, the cells then are administered to a different subject, e.g., a second subject, of the same species. In some embodiments, the first and second subjects are genetically identical. In some embodiments, the first and second subjects are genetically similar. In some embodiments, the second subject expresses the same HLA class or supertype as the first subject.

In some embodiments, the HLA class or HLA supertype of the subject is identified. In some embodiments, the subject is treated with a cell therapy that can recognize the antigen in the context of the HLA class or HLA supertype.

In some embodiments, the subject has been treated with a therapeutic agent targeting the disease or condition, e.g., the tumor, prior to administration of the cells or composition containing the cells. In some aspects, the subject is refractory or non-responsive to the other therapeutic agent. In some embodiments, the subject has persistent or relapsed disease, e.g., following treatment with another therapeutic intervention, including chemotherapy, radiation, and/or hematopoietic stem cell transplantation (HSCT), e.g., allogenic HSCT. In some embodiments, the administration effectively treats the subject despite the subject having become resistant to another therapy.

In some embodiments, the subject is responsive to the other therapeutic agent, and treatment with the therapeutic agent reduces disease burden. In some aspects, the subject is initially responsive to the therapeutic agent, but exhibits a relapse of the disease or condition over time. In some embodiments, the subject has not relapsed. In some such embodiments, the subject is determined to be at risk for relapse, such as at high risk of relapse, and thus the cells are administered prophylactically, e.g., to reduce the likelihood of or prevent relapse. In some embodiments, the subject has not received prior treatment with another therapeutic agent.

In some embodiments, the cells are administered at a desired dosage, which in some aspects includes a desired dose or number of cells or cell type(s) and/or a desired ratio of cell types. Thus, the dosage of cells in some embodiments is based on a total number of cells (or number per kg body weight). In some embodiments, the dosage of cells is based on a desired total number (or number per kg of body weight) of cells in the individual populations or of individual cell types. In some embodiments, the dosage is based on a combination of such features, such as a desired number of total cells, desired ratio, and desired total number of cells in the individual populations.

In some embodiments, the populations of NK cells, such as CD56+ cells, are administered at or within a tolerated difference of a desired dose of total cells. In some embodiments, the desired dose is a desired number of cells or a desired number of cells per unit of body weight of the subject to whom the cells are administered, e.g., cells/kg. In some embodiments, the desired dose is at or above a minimum number of cells or minimum number of cells per unit of body weight.

In some embodiments, the cells are administered at or within a tolerated difference of a desired dose of one or more of the individual populations or sub-types of cells, such as a desired dose of CD56+ cells. In some embodiments, the desired dose is a desired number of cells of the sub-type or population, or a desired number of such cells per unit of body weight of the subject to whom the cells are administered, e.g., cells/kg. In some embodiments, the desired dose is at or above a minimum number of cells of the population or sub-type, or minimum number of cells of the population or sub-type per unit of body weight.

Thus, in some embodiments, the dosage is based on a desired fixed dose of total cells, and/or based on a desired fixed dose of one or more, e.g., each, of the individual sub-types or sub-populations. Thus, in some embodiments, the dosage is based on a desired fixed or minimum dose of NK cells, and/or is based on a desired fixed or minimum dose of CD56+ cells.

In certain embodiments, the cells or individual populations of sub-types of cells, are administered to the subject at a range of about one million to about 100 billion cells, such as, e.g., 1 million to about 50 billion cells (e.g., about 5 million cells, about 25 million cells, about 500 million cells, about 1 billion cells, about 5 billion cells, about 20 billion cells, about 30 billion cells, about 40 billion cells, or a range defined by any two of the foregoing values), such as about 10 million to about 100 billion cells (e.g., about 20 million cells, about 30 million cells, about 40 million cells, about 60 million cells, about 70 million cells, about 80 million cells, about 90 million cells, about 10 billion cells, about 25 billion cells, about 50 billion cells, about 75 billion cells, about 90 billion cells, or a range defined by any two of the foregoing values), and in some cases about 100 million cells to about 50 billion cells (e.g., about 120 million cells, about 250 million cells, about 350 million cells, about 450 million cells, about 650 million cells, about 800 million cells, about 900 million cells, about 3 billion cells, about 30 billion cells, about 45 billion cells) or any value in between these ranges.

In some embodiments, the dose of total cells and/or dose of individual sub-populations of cells is within a range of between at or about 10⁴ and at or about 10⁹ cells/kilograms (kg) body weight, such as between 10⁵ and 10⁶ cells/kg body weight, for example, at least or at least about or at or about 1×10⁵ cells/kg, 1.5×10⁵ cells/kg, 2×10⁵ cells/kg, or 1×10⁶ cells/kg body weight. For example, in some embodiments, the cells are administered at, or within a certain range of error of, between at or about 10⁴ and at or about 10⁹ NK cells/kilograms (kg) body weight, such as between 10⁵ and 10⁶ NK cells/kg body weight, for example, at least or at least about or at or about 1×10⁵ NK cells/kg, 1.5×10⁵ NK cells/kg, 2×10⁵ NK cells/kg, or 1×10⁶ NK cells/kg body weight. In some embodiments, the residual CD3+ cells are administered with less than 2×10⁵ cells/kg, 1×10⁵ cells/kg, 5×10⁴ cells/kg, or 1×10⁴ cells/kg body weight.

In some embodiments, the cells are administered at or within a certain range of error of, greater than, and/or at least about 1×10⁶, about 1×10⁷, about 1×10⁸, about 1×10⁹, or about 1×10¹⁰ CD56+ cells, and/or no greater than about 1×10⁶, about 1×10⁷, about 1×10⁸, about 1×10⁹, or about 1×10¹⁰ CD3+ cells. In some embodiments, the cells are administered at or within a certain range of error of between about 10⁸ and 10¹² or between about 10¹⁰ and 10¹¹ NK cells, between about 10⁸ and 10¹² or between about 10¹⁰ and 10¹¹ CD56+ cells, and/or between about 10⁶ and 10¹⁰ or between about 10⁸ and 10⁹ CD3+ cells.

In some embodiments, the cells are administered at or within a tolerated range of a desired output ratio of multiple cell populations or sub-types, such as CD56+ and CD3+ cells or sub-types. In some aspects, the desired ratio can be a specific ratio or can be a range of ratios. for example, in some embodiments, the desired ratio (e.g., ratio of CD56+ to CD3+ cells) is between at or about 500:1 and at or about 20:1, e.g., about 500:1, about 400:1, about 300:1, about 200:1, about 100:1, about 90:1, about 80:1, about 70:1, about 60:1, about 50:1, about 40:1, about 30:1, or about 20:1. In some aspects, the tolerated difference is within about 1%, about 2%, about 3%, about 4% about 5%, about 10%, about 15%, about 20%, about 25%, about 30%, about 35%, about 40%, about 45%, about 50% of the desired ratio, including any value in between these ranges.

Optimal response to therapy can depend on the ability of the engineered recombinant receptors such as the chimeric polypeptides (or the fusion polypeptide), antibodies or antigen-binding fragments thereof, and/or peptibodies described herein, to be consistently and reliably expressed on the surface of the cells and/or bind the target antigen. For example, in some cases, properties of certain chimeric polypeptides (or the fusion polypeptide), antibodies or antigen-binding fragments thereof, and/or peptibodies, can affect the expression and/or activity of the polypeptide or protein, in some cases when expressed in a cell, such as a human NK cell, used in cell therapy. In some contexts, the level of expression of particular polypeptide or protein, can be low, and activity of the engineered cells, such as human NK cells, expressing such polypeptides or proteins, may be limited due to poor expression or poor signaling activity. In some cases, consistency and/or efficiency of expression of the polypeptide or protein, and activity of the polypeptide or protein is limited in certain cells or certain cell populations of available therapeutic approaches. In some cases, a large number of engineered NK cells (a high effector to target (E:T) ratio) is required to exhibit functional activity. In some embodiments, the desired ratio (E:T ratio) is between at or about 1:10 and at or about 10:1 (or greater than about 1:10 and less than about 10:1), or between at or about 1:1 and at or about 10:1 (or greater than about 1:1 and less than about 5:1), such as between at or about 2:1 and at or about 10:1. In some embodiments, the E:T ratio is greater than or about 1:1, 2:1, 3:1, 4:1, 5:1, 6:1, 7:1, 8:1, 9:1, or 10:1.

For the prevention or treatment of disease, the appropriate dosage may depend on the type of disease to be treated, the type of cells or recombinant receptors, the severity and course of the disease, whether the cells are administered for preventive or therapeutic purposes, previous therapy, the subject's clinical history and response to the cells, and the discretion of the attending physician. The compositions and cells are in some embodiments suitably administered to the subject at one time or over a series of treatments.

The cells described herein can be administered by any suitable means, for example, by bolus infusion, by injection, e.g., intravenous or subcutaneous injections, intraocular injection, periocular injection, subretinal injection, intravitreal injection, trans-septal injection, subscleral injection, intrachoroidal injection, intracameral injection, subconjunctival injection, sub-Tenon's injection, retrobulbar injection, peribulbar injection, or posterior juxtascleral delivery. In some embodiments, they are administered by parenteral, intrapulmonary, and intranasal, and, if desired for local treatment, intralesional administration. Parenteral infusions include intramuscular, intravenous, intraarterial, intraperitoneal, or subcutaneous administration. In some embodiments, a given dose is administered by a single bolus administration of the cells. In some embodiments, it is administered by multiple bolus administrations of the cells, for example, over a period of no more than 3 days, or by continuous infusion administration of the cells.

In some embodiments, the cells are administered as part of a combination treatment, such as simultaneously with or sequentially with, in any order, another therapeutic intervention, such as an antibody or engineered cell or receptor or agent, such as a cytotoxic or therapeutic agent. The cells in some embodiments are co-administered with one or more additional therapeutic agents or in connection with another therapeutic intervention, either simultaneously or sequentially in any order. In some contexts, the cells are co-administered with another therapy sufficiently close in time such that the cell populations enhance the effect of one or more additional therapeutic agents, or vice versa. In some embodiments, the cells are administered prior to the one or more additional therapeutic agents. In some embodiments, the cells are administered after the one or more additional therapeutic agents. In some embodiments, the one or more additional agents includes a cytokine, such as IL-2, for example, to enhance persistence. In some embodiments, the methods comprise administration of a chemotherapeutic agent.

Following administration of the cells, the biological activity of the engineered cell populations in some embodiments is measured, e.g., by any of a number of known methods. Parameters to assess include specific binding of engineered T cells to the antigen, in vivo, e.g., by imaging, or ex vivo, e.g., by ELISA or flow cytometry. In certain embodiments, the ability of the engineered cells to destroy target cells can be measured using any suitable method known in the art, such as cytotoxicity assays described in, for example, Kochenderfer et al. “Construction and pre-clinical evaluation of an anti-CD19 chimeric antigen receptor.” Journal of immunotherapy (Hagerstown, Md.: 1997) 32.7 (2009): 689 and Hermans et al. “The VITAL assay: a versatile fluorometric technique for assessing CTL- and NKT-mediated cytotoxicity against multiple targets in vitro and in vivo.” Journal of immunological methods 285.1 (2004): 25-40. In certain embodiments, the biological activity of the cells is measured by assaying expression and/or secretion of one or more cytokines, such as CD107a, IFNγ, IL-2, and TNF. In some aspects the biological activity is measured by assessing clinical outcome, such as reduction in tumor burden or load.

### EXAMPLES

The invention is further described in the following examples, which do not limit the scope of the invention described in the claims.

### Example 1: Generation of Pluck-NK Cells

In the following Examples, the Pluck-NK cells according to the embodiments as described above and illustrated in FIG. 6 and FIG. 7, i.e., cord-blood derived NK cells equipped with Recast-IL15 and the self-secreting full-length anti-PD-L1 antibody carrying an engineered Fc region, were substantially established and tested.

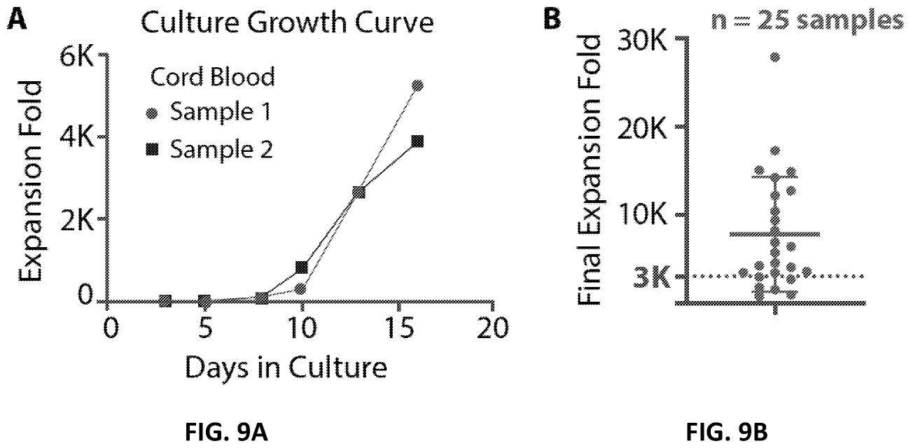

Mononuclear cells from cord blood were cultured with support of an engineered feeder cell line on Day 0. Transduction was performed at a selected time point during the 2-week cell culture. The matured product cells were harvested on Day 14. FIGS. 9A-9B show the cell growth curves of two representative cord blood samples (FIG. 9A) and cell expansion fold of 25 cord blood samples on Day 14 (FIG. 9B). On average, cord blood-derived NK cells, after virus transduction, can be expanded more than 3,000 folds in vitro on Day 14. This in vitro expansion capacity is comparable to, if not better than, the benchmark report (Liu E et al. Use of CAR-Transduced Natural Killer Cells in CD19-Positive Lymphoid Tumors. N Engl J Med 2020; 382:545-553; or “Liu et al. 2020”).

### Example 2: Cell Purity

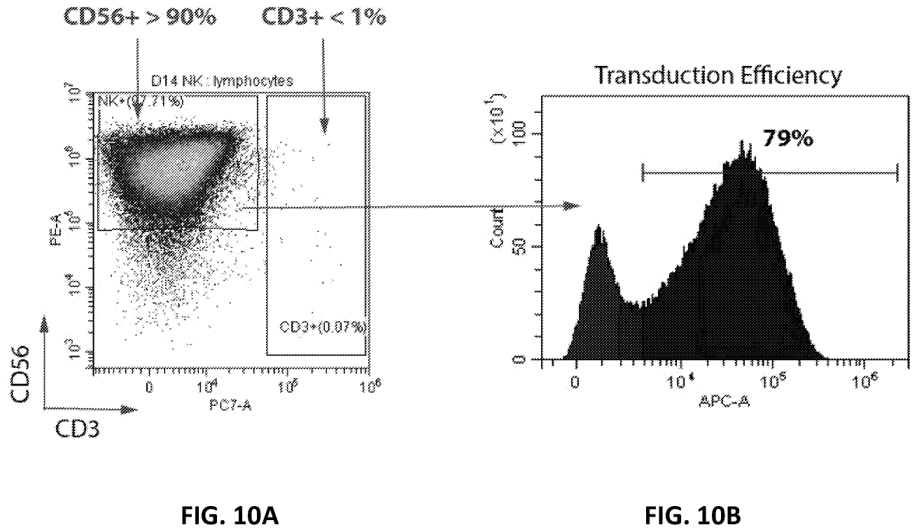

The product cells included more than 90% of NK cells (CD56⁺) and less than 1% of residual T cells (CD3⁺) (FIG. 10A). These parameters are comparable to our benchmark (Liu et al. 2020). The residual CD3⁺ cells is also one determining factor of the drug's infusion dosage. CD3⁺ cells are not compatible for allogeneic usage. The safety standard for allogeneic CD3+ cell infusion is less than 2× 105 per kg body weight (Liu et al. 2020). For a safe estimation on the infusion dosage of Pluck-NK, if the body weight of a patient is 50 kg and 1% residual CD3⁺ cells are in the final product, the maximum infusion dosage for the patient is 1×10⁹ Pluck-NK cells. In fact, 1×10⁹ cells per patient fall within the high dosage range in immune cell therapy. Thus, for higher infusion dosage, one can consider multiple rounds of infusion as an option.

### Example 3: Transduction Efficiency

Pluck-NK cells were transduced by a retroviral vector. Transduction efficiency ranged from 70% to 90% when fresh virus was used (FIG. 10B). The transduction efficiency may drop when frozen virus produced by a stable producer cell line was used. We set the product release criteria (or transduction efficiency indicating the percentage of cells being successfully transduced by the retroviral vector; details can be found in Liu et al. 2020) at 20%. In the benchmark report, the transduction efficiency for the CAR-NK products ranged from 22% to 67% with the product release criteria of 15% (Liu et al. 2020).

### Example 4: Productivity of Pluck-NK Cells

One cord blood sample was about 50 ml carrying about 1× 108 white blood cells. 5-30% of the white blood cells were NK cells. Considering the cell expansion capacity, transduction efficiency, cell purity and cell viability after cryopreservation of the current NK cell platform, it is estimated that 10 billion drug cells (1010) can be produced from each cord blood sample.

### Example 5: Functionality Assessment of Pluck-NK

To assess the functionality of Pluck-NK cells, we studied Pluck-NK's cytokine profile upon NK cell activation, performed target-cell killing assay, monitored long-term Pluck-NK growth in culture without supporting cytokines, monitored the anti-PD-L1 antibody production, and tested the cryopreservability. Each experiment is described as follows.

### Cytokine Profiles Upon NK Cell Activation

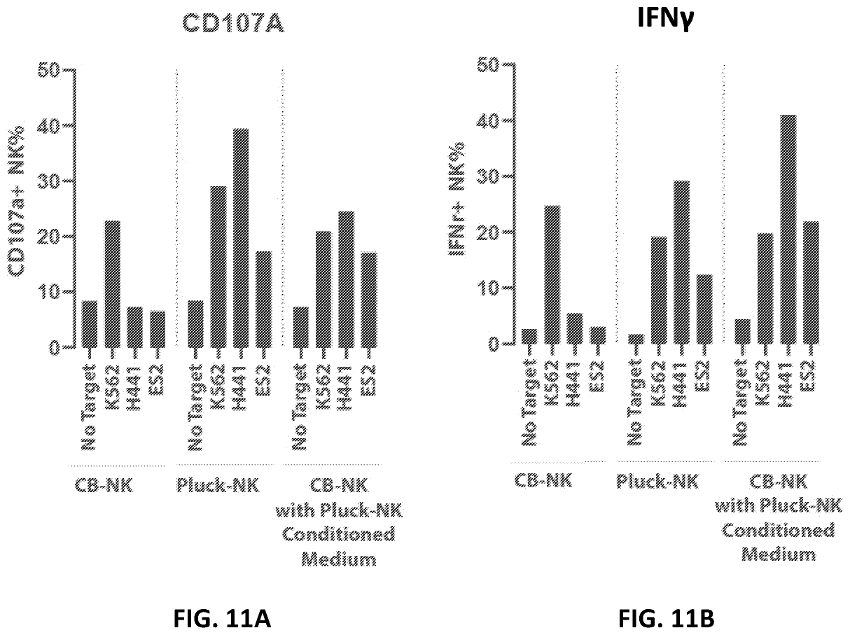

To assess Pluck-NK's functionality, intracellular staining of IFNγ and CD107A, two cytokines related to NK cell activation, was performed, followed by fluorescence-activated cell sorting (FACS). CD107a and INFγ are two important cytokines produced by NK cells upon activation, whose production is tightly linked to NK cell activation. Three target cell lines were used in the experiments shown in FIGS. 11A-11B. K562 cells are cancer cells not expressing the MHC class I antigen. Therefore, K562 cells can be recognized and killed by all mature NK cells with a miss-self killing capacity, including untransduced NK cells from the cord blood (CB-NK cells) and Pluck-NK cells. ES-2 (ovarian cancer) and H441 (lung cancer) are two cancer cell lines expressing PD-L1 at the cell surface. Therefore, Pluck-NK cells should be able to recognize and kill H441 and ES-2 cells by anti-PD-L1 ADCC. To further investigate and provide direct evidence on anti-PD-L1 ADCC, we used the medium conditioned by Pluck-NK to treat untransduced CB-NK cells. The conditioned medium contained no cells but the cell secretion products including the anti-PD-L1 antibody. Through ADCC, the conditioned medium can grant CB-NK cells the ability to recognize and kill H441 and ES-2 cells. As shown in FIGS. 11A-11B, the results show that Pluck-NK cells can recognize and kill all three target cell lines. Moreover, the conditioned medium, containing the anti-PD-L1 antibody secreted by Pluck-NK cells, enabled CB-NK cells to recognize and kill H441 and ES-2 cells, providing the direct evidence of anti-PD-L1 ADCC of Pluck-NK cells.

### Cell Killing Assay

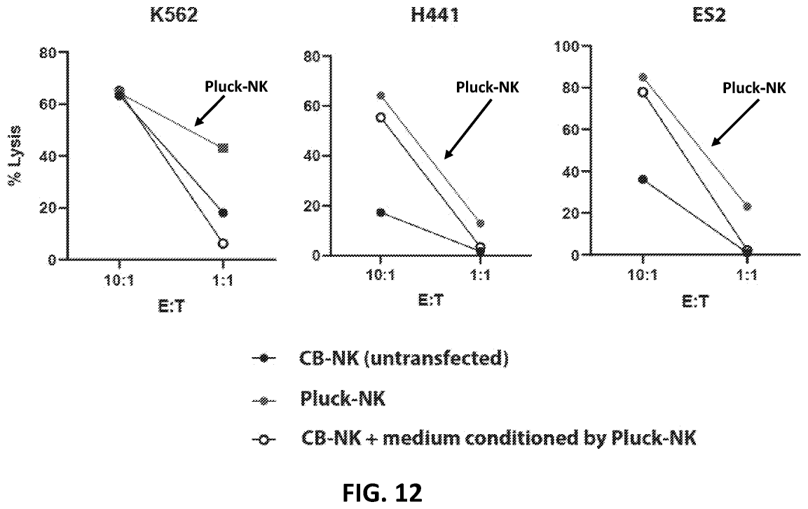

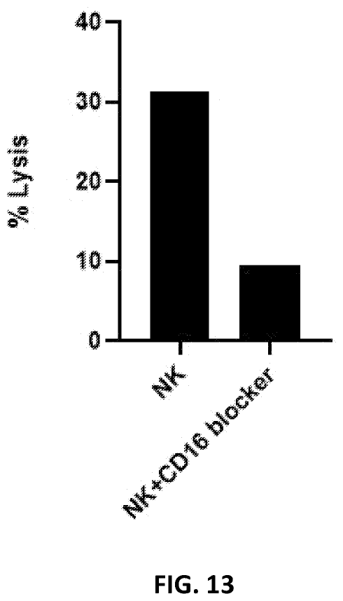

To assess Pluck-NK's functionality, cell killing assays were performed. The same three target cell lines described above were used in the cell killing assay to directly monitor target cell killing. The conditioned medium was also used in the cell killing assay to collect direct evidence of anti-PD-L1 ADCC. As shown in FIG. 12, the results confirmed that Pluck-NK cells can recognize and kill all three target cell lines. Moreover, the killing of H441 and ES-2 were mediated by anti-PD-L1 ADCC, as evidenced by the effect of the conditioned medium (FIG. 12) and the Fc blocker (FIG. 13).

### Autonomous Growth of Pluck-NK in Culture

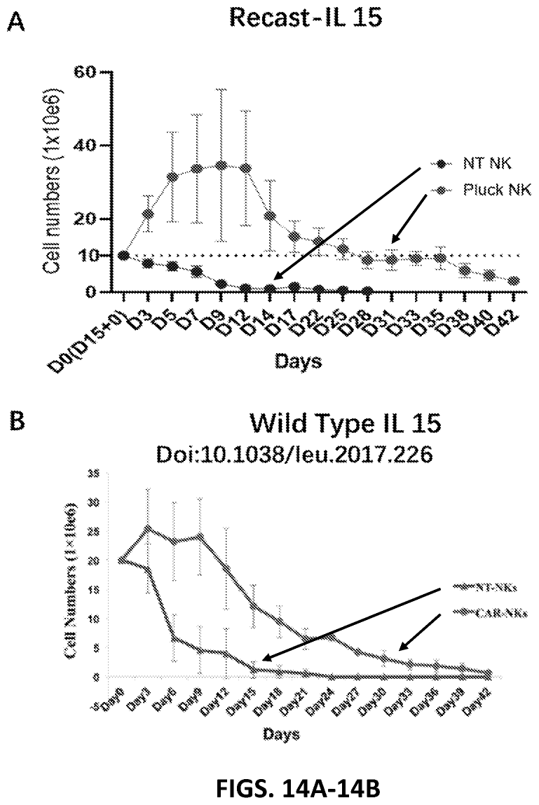

Previously, our data (Liu, E., et al. 2020) showed that the secretion of wildtype IL15 cytokine confers some level of autonomous growth capability to the NK cells cultured in vitro in the absence of any cytokine supplements. As shown in FIG. 14B, the wildtype IL15-secreting CAR-NK cells exhibited relatively better persistence than the control NT-NK cells (i.e., non-transduced NK cells) when these two populations of cells are cultured in vitro without any cytokine supplements. Specifically, the total cell number of the IL15-secreting CAR-NK cells remained higher than that on Day 0 for about 9 days, then starting on Day 12 the total cell number became lower than that on Day 0, and after almost one month (i.e., 27-30 days) of culture, only about 25% of the CAR-NK cells left in the culture compared with Day 0. In contrast, NK cells not equipped with the self-secreting IL15 that were cultured under the same condition did not show any signs of persistency or cell expansion starting from Day 0, and the cell population drastically reduced over time and vanished after about two weeks in culture (FIG. 14B).

The autonomous growth capability that is conferred by the Recast-IL15 to the NK cells cultured in vitro without any supporting cytokines or feeder cells was further evaluated. CB-NK, the untransduced NK cells, were also monitored under the same experimental conditions as a control. In this experiment, the starting populations of Pluck-NK and CB-NK were both matured cell products harvested from the Day 14 culture. Interestingly and unexpectedly, compared with the secreted wildtype IL15 (data shown in FIG. 14B), the Recast-IL15 can support much longer term of autonomous growth of the Pluck-NK cells when cultured in vitro without any supporting cytokines, as shown in FIG. 14A. More specifically, after the cytokine supplement-free in vitro culture started, the total cell number of the Pluck-NK cells increased starting from Day 0 and almost tripled on Day 9, and subsequently, the total cell number remained consistently higher than that on Day 0 even after approximately 25 days of culture, and then remained substantially unchanged (i.e., the total cell number is no less than 90%) compared with Day 0 after approximately 28-35 days of culture (FIG. 14A). By contrast, the control CB-NK cells that were cultured in the same condition started reducing in cell number even on Day 0, and almost died out after 9 days (FIG. 14A). This results indicate the apparent function of Recast-IL15 in enhancing NK cells' persistence.

Thus in terms of the effects of the Recast-IL15 and the secreted wildtype IL15 in sustaining NK cells' persistence, although they are expected to mechanistically function on the same signaling pathway, the membrane-bound Recast-IL15 confers the NK cells an unexpectedly better capability of autonomous growth when cultured in vitro in the absence of any cytokine supplements (e.g., IL-2, IL-15, IL-21, etc.), compared with the secreted wildtype IL15. While the secreted wildtype IL15 can only support the autonomous growth of NK cells for approximately 10 days (i.e., the total cell number of IL15-secreting NK cells remains no less than that on Day 0 of culture), the Recast-IL15 can unexpectedly support the more favorable long-term autonomous growth of NK cells cultured in an in vitro and cytokine supplement-free condition, with their total cell number remaining no less than that on Day 0 within almost one month of culture (i.e., the total cell number of the Recast-IL15 expressing NK cells remained more than, or substantially equal (i.e., no less than 90%) to, the total cell number of the cells on Day 0).

### Anti-PD-L1 Antibody Production

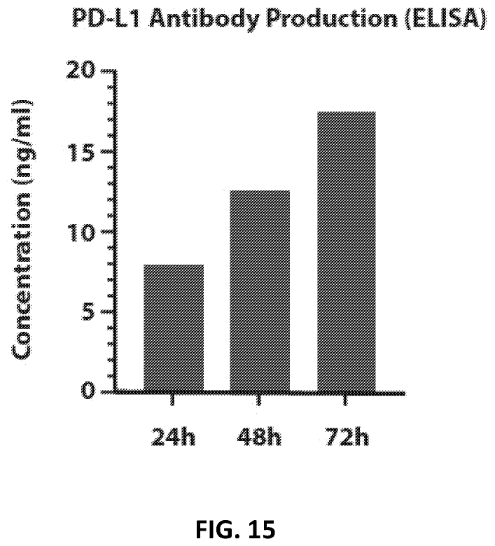

The production of the anti-PD-L1 antibody by Pluck-NK cells was tested by ELISA, and the results are shown in FIG. 15. Briefly, 1×10⁶ drug cells were seeded in each well of a 24-well plate with 2 ml culture medium. Anti-PD-L1 antibody secretion was detected by ELISA using the cell culture medium at three selected time points (24 hours, 48 hours, and 72 hours after seeding).

### Resistance of Pluck-NK Cells to Cryopreservation-Induced Apoptosis

Cryopreservation can induce apoptosis of immune cells, and this phenomenon may attenuate the therapeutic efficacy of adoptive cell therapy. To investigate whether cryopreservation can induce apoptosis on Pluck-NK cells, the apoptosis assay was performed on fresh and cryopreserved Pluck-NK cells. Specifically, Pluck-NK cells were recovered from cryopreservation and incubated in a cell culture medium for 2 hours. 7-AAD (7-Aminoactinomycin D) is a dye that binds to DNA but can be efficiently excluded by intact cells. Annexin-V is commonly used for staining apoptotic cells. Thus, Annexin-V⁺ and AAD⁻ cells are those with early onset of apoptosis and good cell membrane integrity.

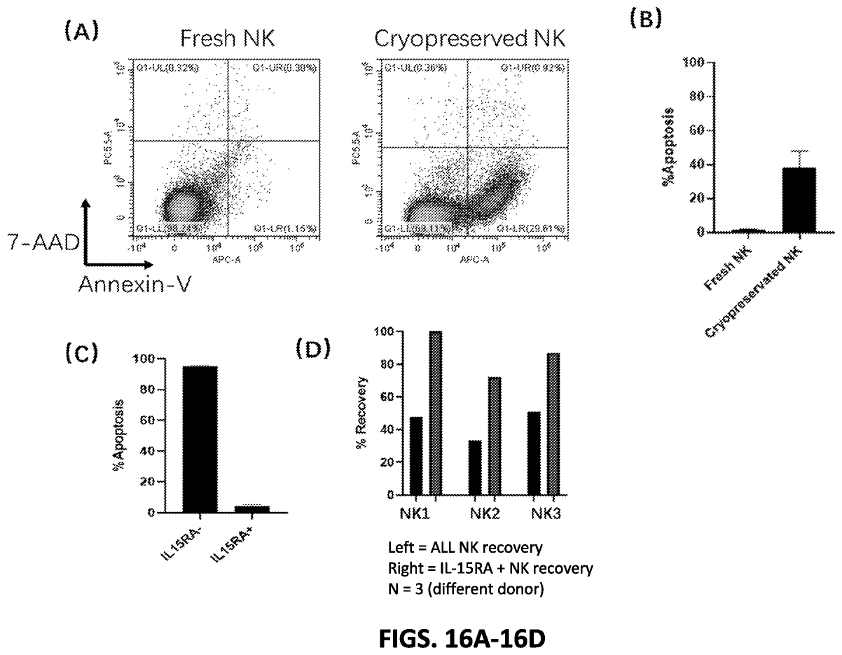

Results showed that cryopreservation caused apoptosis (Annexin-V⁺ 7-AAD⁻) in about 35% Pluck-NK cells (FIGS. 16A-16B). Further analysis revealed that within the apoptotic NK cell population, majority of cells (>95%) didn't carry transgene (IL15RA⁻), and therefore were not Pluck-NK cells (FIG. 16C). Only about 5% of apoptotic NK cells were Pluck-NK cells (IL15RA⁺), which also showed a higher recovery rate as compared to all the apoptotic NK cells (FIG. 16D). The results indicate that although cryopreservation-induced apoptosis was observed in normal NK cells (NK cells not carrying transgene), Pluck-NK cells with Recast-IL15 resisted to this apoptotic process.

### Example 6: In Vivo Safety and Efficacy

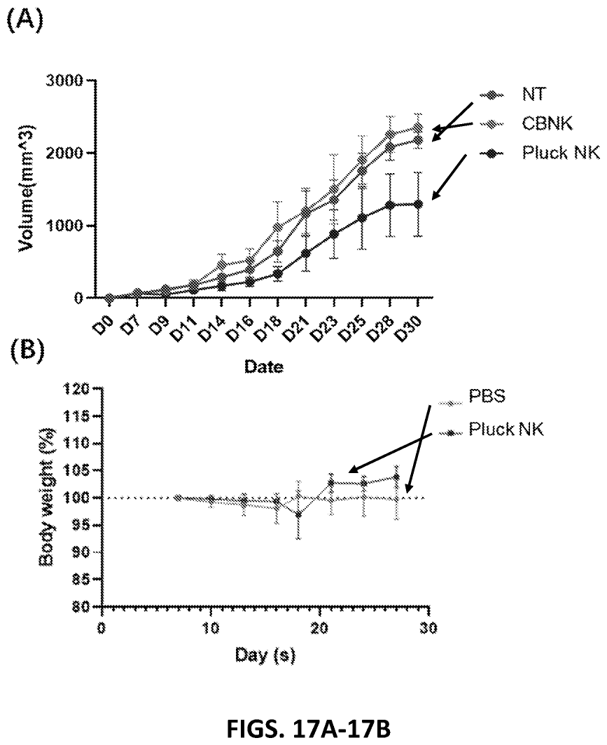

Animal experiments were performed to study the in vivo efficacy and safety of cryopreserved Pluck-NK cells using NCG (NOD/ShiLtJGpt-Prkdc^{em26Cd52}Il2rg^{em26Cd22}/Gpt) mice (Gempharmatech strain NO.T001475). Tumorgenesis was performed by subcutaneous injection of 1×10⁷ CaSki cells. Seven days after tumorgenesis, drug cell infusion was performed by intravenous (IV) injection of 1×10⁷ Pluck-NK cells into the lateral tail veins of adult mice. Tumor sizes were monitored to assess in vivo efficacy (FIG. 17A). For in vivo safety, the body weight of mice injected with Pluck-NK cells was monitored and compared with mice injected with PBS (FIG. 17B). The results confirmed that the in vivo efficacy and safety of Pluck-NK. By design, Pluck-NK cells can trigger multiple layers of immune responses, including miss-self killing, ADCP, CDC and immune checkpoint blockade (ICB). Only ADCC was assessed in these animal experiments.

### Example 7: Preparation of Peptibody-NK Cells

To prove the concept that the engineered therapeutic NK cells can be specifically directed to target cancer cells by engineered ADCC, an exemplary peptibody-NK design is illustrated here. A peptibody is a type of protein that includes a biologically active peptide fused to an Fc region of an antibody, and the Fc region is responsible for the long half-life and high affinity of antibodies, which makes peptibodies attractive for therapeutic applications. In this specific example, the target binding domain of the anti-IL13Rα2 peptibody comprises an interleukin IL13 (E13Y) ligand or a functional fragment or variant thereof, that allows the peptibody secreted from the NK cells to selectively binds to the receptor IL13Rα2 that is found to be restrictedly expressed in the malignant glioma and renal cell carcinoma cells.

Specifically, the IL13 (E13Y) ligand (SEQ ID NO: 22) was fused with the Fc region (SEQ ID NO: 24) to generate a secreted fusion protein (i.e., “IL13-Fc” hereinafter). The sequence set forth in SEQ ID NO: 25 provides the full-length fusion protein with a signal peptide SEQ ID NO: 8). The IL13 (E13Y) ligand provides the targeting mechanism for the IL13Rα2⁻ target cells. IL13-Fc NK cells were derived from cord blood and were equipped with Recast-IL15 and the IL13-Fc peptibody (Table 5).

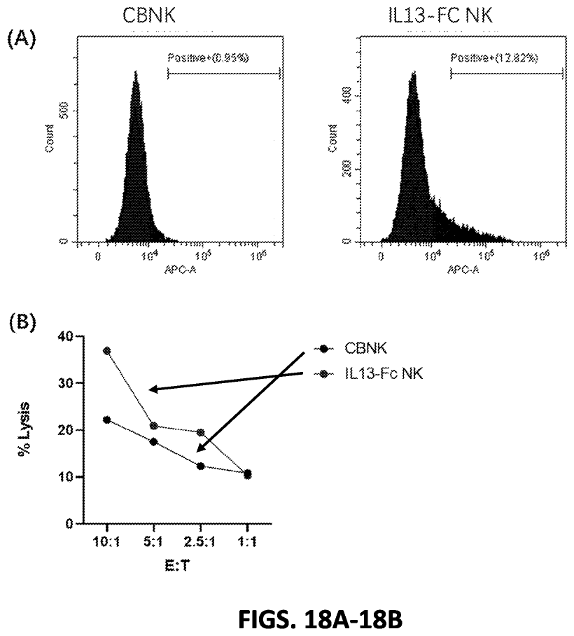

The IL13-Fc transgenes were successfully inserted into cord blood-derived NK cells (FIG. 18A). To validate the therapeutic efficacy of IL13-Fc NK, CB-NK (untransduced NK cells) and IL13-Fc NK cells were co-cultured with IL13Rα2⁺ glioblastoma cell line U251 at different effector: target ratios (10:1, 5:1, 2.5:1, or 1:1), respectively. Results showed that the cytotoxicity of IL13-Fc NK was stronger than CB-NK, particularly at the 10:1 ET ratio, demonstrating that self-secreting anti-IL13Rα2 peptibody enhanced NK's activity against glioblastoma cell line U251 (FIG. 18B). However, with the current transduction efficiency, clear results were obtained to prove the concept that the secreted IL13-Fc peptibody guided the NK cells to kill tumor cells (FIG. 18B).

### Example 8: Characterization of Anti-PD-L1 Antibodies

### Cellular Binding Assay

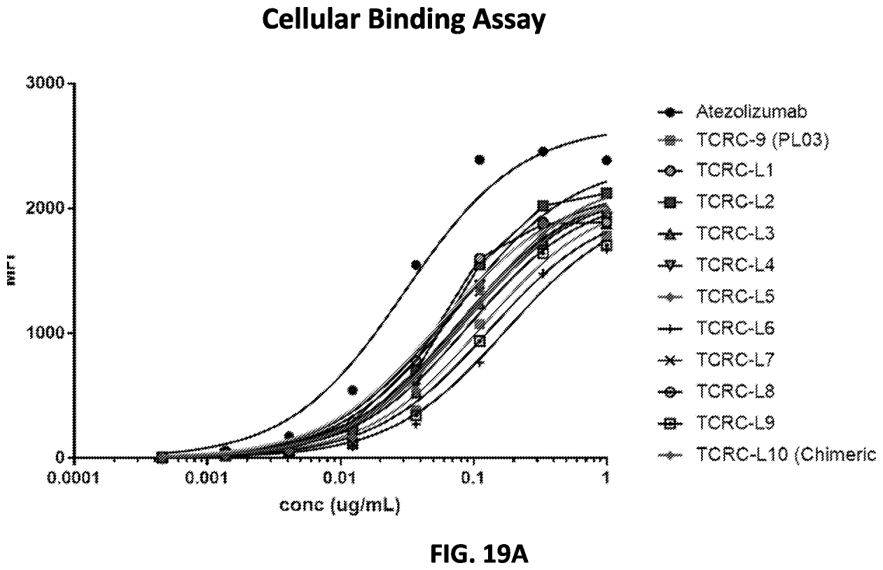

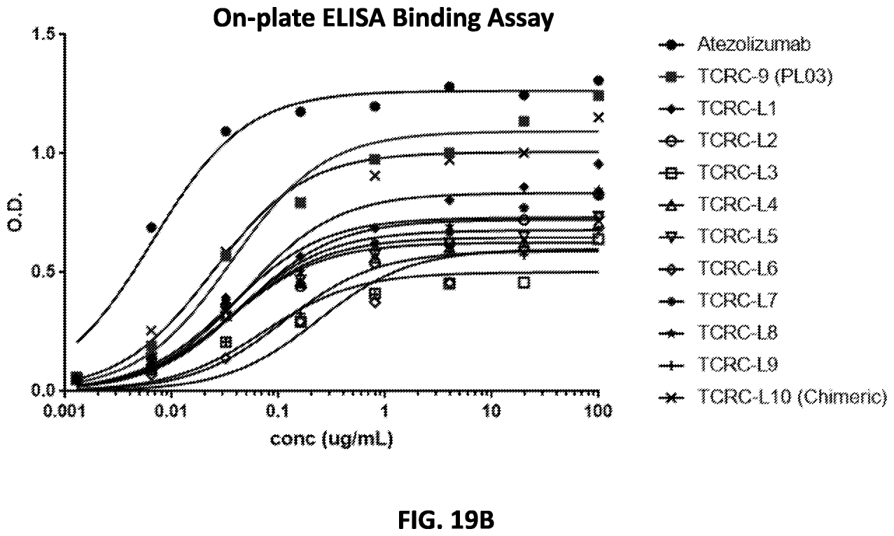

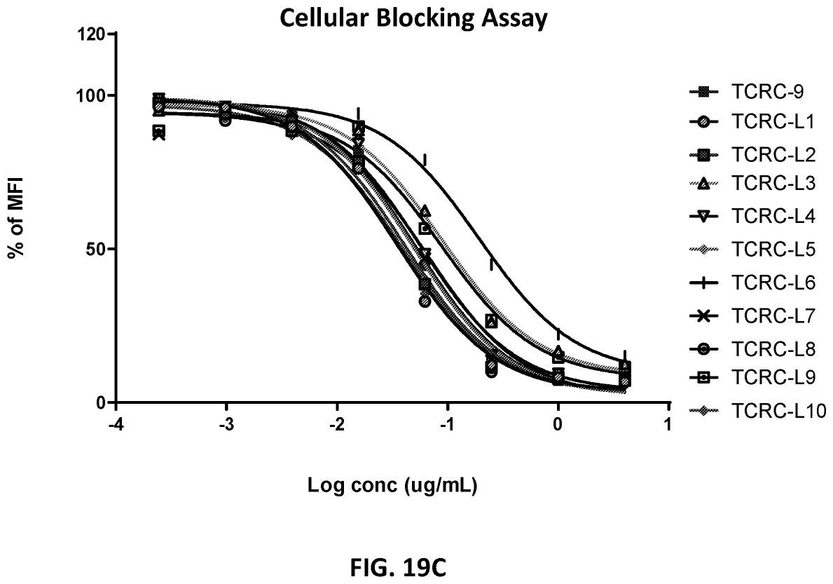

Cells expressing PD-L1 (0.03× 106/well) were incubated with different concentrations of anti-PD-L1 antibodies including atezolizumab (Ate), TCRC-L1, TCRC-L2 (or the L2 clone described above), TCRC-L4, TCRC-L5, TCRC-L7, TCRC-L8 (or the L8 clone described above), and TCRC-L10 at 4° C. for 60 minutes. The cells were then stained with secondary antibodies for 30 minutes (1:50) (APC-anti-Hu IgG Fc) and followed by flow cytometric analysis. The results were analyzed through Prism 7 and Kd values were determined by the program. The results are shown in FIG. 19A, and Kd values are shown in the table below.

TABLE 6
Kd determined by the cellular binding assay

| Antibody | Kd (μg/mL) |
|---|---|
| Ate | 0.03 |
| TCRC-L1 | 0.053 |
| TCRC-L2 | 0.074 |
| TCRC-L4 | 0.078 |
| TCRC-L5 | 0.088 |
| TCRC-L7 | 0.083 |
| TCRC-L8 | 0.058 |
| TCRC-L10 | 0.088 |

### On-Plate ELISA Binding Assay

0.2 μg/ml of recombinant human PD-L1 (rhPD-L1) protein in 100 μl coating buffer was coated in the microwells at 4° C. overnight. By following the ELISA procedure, a 5-fold serial dilution of anti-PD-L1 antibodies were added to the assay plate pre-coated with rhPD-L1. The bound anti-PD-L1 antibody was then detected with HRP-conjugated anti-IgG-Fc reagent. The results were analyzed through Prism 7 and Kd values were determined by the program. The results are shown in FIG. 19B, and Kd values are shown in the table below.

TABLE 7
Kd determined by the on-plate ELISA binding assay

| Antibody | Kd (μg/mL) |
|---|---|
| Ate | 0.0065 |
| TCRC-L1 | 0.047 |
| TCRC-L2 | 0.04 |
| TCRC-L4 | 0.035 |
| TCRC-L5 | 0.037 |
| TCRC-L7 | 0.046 |
| TCRC-L8 | 0.036 |
| TCRC-L10 | 0.024 |

### Cellular Blocking Assay

Cells expressing PD-L1 (0.03e6/well) were incubated with 1 μg/ml of Biotin-PD-1 and 5 μg/ml of anti-PD-L1 antibodies (4-fold dilution) at 4° C. for 30 minutes. Afterwards, the cells were stained with secondary antibodies for 30 minutes (PE-Streptavidin). The results are shown in FIG. 19C, and EC₅₀ values are shown in the table below.

TABLE 8
EC₅₀ determined by the cellular blocking assay

| Antibody | EC₅₀ (μg/mL) |
|---|---|
| Ate | 0.023 |
| TCRC-L1 | 0.053 |
| TCRC-L2 | 0.074 |
| TCRC-L4 | 0.078 |
| TCRC-L5 | 0.088 |
| TCRC-L7 | 0.083 |
| TCRC-L8 | 0.058 |
| TCRC-L10 | 0.088 |

### Example 9: Binding Specificity of Anti-PD-L1 Antibody TCRC-L8

### MPA Screen

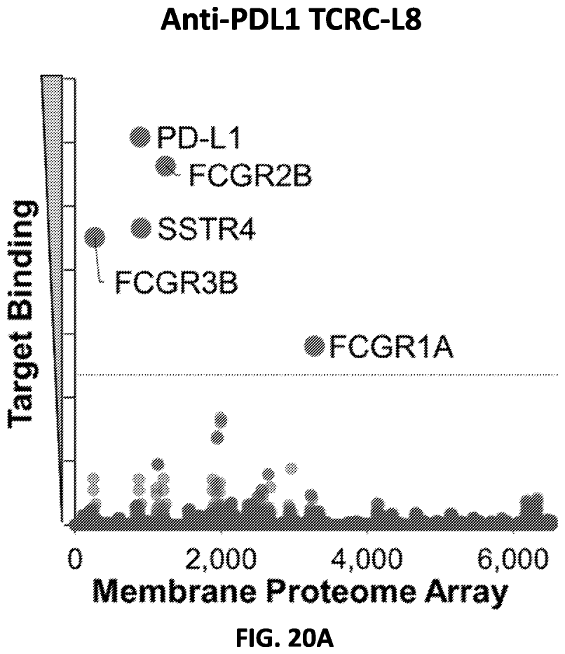

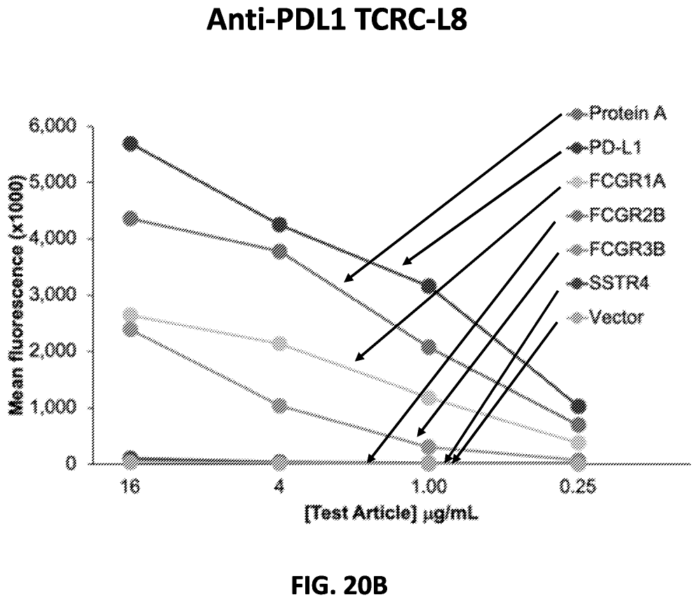

As shown in FIG. 20A, the test article was screened on the MPA (Membrane Proteome Array) using conditions determined from the assay setup. The MPA was expressed in HEK-293T cells and test article binding was determined by flow cytometry. Each target was tested for binding in duplicate. Anti-PD-L1 TCRC-L8 showed binding to the known binding partner PD-L1. The test article also bound three FCGR proteins (FCGR2B, FCGR3B, and FCGR1A), which was expected because FCGR proteins can bind human Fc and serve as positive controls in the assay. The MPA screen identified SSTR4 as a binding interaction that was validated in titration studies. Any potential binding interactions identified on the MPA that did not validate in titration experiments were removed from the graph. Dotted line represents 3 SD of the calculated background. The results indicate that TCRC-L8 might bind to SSTR4 (off-target).

### Validation by Titration Experiments

As shown in FIG. 20B, anti-PD-L1 TCRC-L8 showed strong binding to the positive controls Protein A and PD-L1, with MFI signals >106-fold and >138-fold above the negative control, respectively. Anti-PD-L1 TCRC-L8 also bound three different FCGR proteins with MFI signal >2 fold above the negative control (FCGR1A 64-fold, FCGR3B 52-fold, and FCGR2B 2-fold). Binding to FCGR proteins was expected because the test article contains human Fc protein which FCGR proteins can bind. The binding of anti-PD-L1 TCRC-L8 to SSTR4 is pretty weak in this validation assay setup, compared to its cognate receptor, PD-L1. As a conclusion, the results in the MPA screen assay were validated by the titration experiment, indicating that TCRC-L8 does not bind to SSTR4 and can bind to PD-L1 specifically.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An engineered immune cell, expressing a chimeric polypeptide comprising an interleukin 15 (IL15) domain and an interleukin 15 receptor, alpha subunit (IL15Rα) domain, wherein: when expressed, the chimeric polypeptide is cell membrane-bound, and when a population of the engineered immune cell is cultured in vitro, the total cell number thereof remains higher or substantially unchanged after at least 7 days of culture without any supporting cytokines.

2. The engineered immune cell of claim 1, wherein the immune cell is a natural killer (NK) cell, a T cell, or a tumor-infiltrating cell (TIL), preferably a NK cell.

3. The engineered immune cell of claim 1 or 2, wherein the engineered immune cell is an NK cell.

4. The engineered immune cell of any one of claims 1-3, wherein the IL15 domain has an amino acid sequence that is at least 90%, at least 95%, at least 96%, at least 97%, at least 98%, or at least 99% identical to SEQ ID NO: 2.

5. The engineered immune cell of claim 4, wherein the IL15 domain comprises an Asp residue at a position corresponding to Asn72 of SEQ ID NO: 2.

6. The engineered immune cell of claim 5, wherein the IL15 domain has an amino acid sequence that is at least 90%, at least 95%, at least 96%, at least 97%, at least 98%, or at least 99% identical to SEQ ID NO: 3.

7. The engineered immune cell of any of claims 1-6, wherein the IL15Rα domain has an amino acid sequence that is at least 90%, at least 95%, at least 96%, at least 97%, at least 98%, or at least 99% identical to SEQ ID NO: 5.

8. The engineered immune cell of any of claims 1-7, wherein the IL15 domain and the IL15a domain are fused via an engineered linker, wherein the engineered linker comprises an amino acid sequence that is at least 90%, at least 95%, at least 96%, at least 97%, at least 98%, or at least 99% identical to SEQ ID NO: 4.

9. The engineered immune cell of any of claims 1-8, wherein the chimeric polypeptide comprises an amino acid sequence that is at least 90%, at least 95%, at least 96%, at least 97%, at least 98%, or at least 99% identical to SEQ ID NO: 6 or SEQ ID NO: 7.

10. The engineered immune cell of any of claims 1-9, when a population of the immune cell is cultured in vitro, the total cell number thereof is higher or remains substantially unchanged after at least 35 days of culture without any supporting cytokines.

11. The engineered immune cell of any of claims 2-10, wherein the NK cell is obtained from cord blood.

12. The engineered immune cell of any of claims 1-11, wherein the engineered immune cell further expresses and/or secrets one or more polypeptides, each comprising a target-binding domain, wherein the target-binding domain is capable of specifically recognizing and binding to a target molecule expressed or presented on the cell surface of a target cell.

13. The engineered immune cell of claim 12, wherein each of the one or more polypeptides further comprises an Fc domain, wherein the Fc domain is capable of mediating antibody-dependent cellular cytotoxicity (ADCC) for the engineered immune cell.

14. The engineered immune cell of claim 13, wherein the Fc domain comprises at least one of Ser239Asp, Ala330Leu, Ile332Glu, Phe243Leu, Arg292Pro, Tyr300Leu, Val305Ile, Pro396Leu, Met428Leu, or Asn434Ser according to EU numbering.

15. The engineered immune cell of claim 14, wherein the Fc domain comprises at least one set of:
(a) Ser239Asp, Ala330Leu and Ile332Glu;
(b) Phe243Leu, Arg292Pro, Tyr300Leu, Val305Ile and Pro396Leu; or
(c) Met428Leu and Asn434Ser.

16. The engineered immune cell of claim 14 or 15, wherein the Fc domain comprises a combination of Ser239Asp, Ala330Leu, Ile332Glu, Phe243Leu, Arg292Pro, Tyr300Leu, Val305Ile, Pro396Leu, Met428Leu and Asn434Ser.

17. The engineered immune cell of any of claims 13-16, wherein each of the one or more polypeptides further comprises a first masking peptide fused at the N-terminal of the antigen-binding domain or the C-terminal of the Fc domain via a first flexible linker, wherein the first masking peptide is configured to block the Fc domain of the polypeptide, and the first flexible linker is configured to be cleavable in a target tissue.

18. The engineered immune cell of claim 17, wherein the target tissue is a tumor tissue, and the first flexible linker is configured to be degradable by matrix metalloproteinase 1 (MMP1), matrix metalloproteinase 2 (MMP2), matrix metalloproteinase 3 (MMP3), matrix metalloproteinase 7 (MMP7), matrix metalloproteinase 9 (MMP9), and/or matrix metalloproteinase 14 (MMP14).

19. The engineered immune cell of claim 18, wherein the first flexible linker is configured to be degradable by MMP9, and the first flexible linker has an amino acid sequence selected from SEQ ID NO: 26 or SEQ ID NO: 27.

20. The engineered immune cell of claim 17, wherein the first flexible linker is configured to be degradable by a proteinase (protease) that is exogenously provided to the target tissue.

21. The engineered immune cell of any of claims 13-20, wherein each of the one or more polypeptides further comprises a second masking peptide fused at the N-terminal of the antigen-binding domain or the C-terminal of the Fc domain via a second flexible linker, wherein the second masking peptide is configured to block the antigen-binding domain of the polypeptide and the second flexible linker is configured to be cleavable in a target tissue.

22. The engineered immune cell of claim 21, wherein the target tissue is a tumor tissue, and the second flexible linker is configured to be degradable by MMP1, MMP2, MMP3, MMP7, MMP9 or MMP14.

23. The engineered immune cell of claim 22, wherein the second flexible linker is configured to be degradable by MMP9, and the second flexible linker has an amino acid sequence selected from SEQ ID NO: 26 or SEQ ID NO: 27.

24. The engineered immune cell of claim 21, wherein the second flexible linker is configured to be degradable by a proteinase (protease) that is exogenously provided to the target tissue.

25. The engineered immune cell of any of claims 12-24, wherein the one or more polypeptides comprises an antibody or antigen-binding fragment thereof, wherein the target-binding domain is an antigen-binding domain, comprising a single-chain variable fragment (scFv) domain or a single monomeric variable antibody domain.

26. The engineered immune cell of claim 20, wherein the target molecule is selected from a group consisting of PD-L1, BCMA, Caudin18.2, CCR4, CD10, CD123, CD147, CD171, CD19, CD20, CD22, CD276, CD319, CD33, CD38, CD70, CLL-1, DLL3, EGFR, EGFRvIII, EpCAM, FLT3, FRα, GD2, GPC3, HER2, HGFR, IL13Ralpha2, Mesothelin, MG7, MUC1, MUC16, Nectin4, PSCA, ROR1, ROR2, TACI, TRBC1, TSLPR, and VEGFR.

27. The engineered immune cell of claim 25 or 26, wherein the antibody or antigen-binding fragment thereof is an anti-PD-L1 antibody or antigen-binding fragment thereof.

28. The engineered immune cell of claim 27, wherein the anti-PD-L1 antibody or antigen-binding fragment thereof comprises an scFv domain, wherein the scFv domain comprises a VL domain having an amino acid sequence that is at least 90%, at least 95%, at least 96%, at least 97%, at least 98%, or at least 99% identical to SEQ ID NO: 9, and/or comprises a VH domain having an amino acid sequence that is at least 90%, at least 95%, at least 96%, at least 97%, at least 98%, or at least 99% identical to SEQ ID NO: 10.

29. The engineered immune cell of claim 28, wherein the scFv domain of the anti-PD-L1 antibody or antigen-binding fragment thereof comprises an amino acid sequence that is at least 90%, at least 95%, at least 96%, at least 97%, at least 98%, or at least 99% identical to SEQ ID NO: 12.

30. The engineered immune cell of claim 27, wherein the anti-PD-L1 antibody or antigen-binding fragment thereof comprises an scFv domain, wherein the scFv domain comprises a VL domain having an amino acid sequence that is at least 90%, at least 95%, at least 96%, at least 97%, at least 98%, or at least 99% identical to SEQ ID NO: 13, and/or comprises a VH domain having an amino acid sequence that is at least 90%, at least 95%, at least 96%, at least 97%, at least 98%, or at least 99% identical to SEQ ID NO: 14.

31. The engineered immune cell of claim 30, wherein the scFv domain of the anti-PD-L1 antibody or antigen-binding fragment thereof has an amino acid sequence that is at least 90%, at least 95%, at least 96%, at least 97%, at least 98%, or at least 99% identical to SEQ ID NO: 15.

32. The engineered immune cell of any of claims 12-24, wherein the one or more polypeptides comprises a peptibody, wherein the target-binding domain comprises a ligand polypeptide.

33. The engineered immune cell of claim 32, wherein the target molecule is a receptor that the ligand polypeptide is capable of specifically recognizing and binding, wherein the receptor is selected from a group consisting of IL13Rα2, APN/CD13, APP, PD-L1, CD44, P32/gC1qR, E-cadherin, N-cadherin, CD21, EGFR, Epha2, EphB4, HER2, FGFR1, FGFR2, FGFR3, FGFR4, VEGFR1, VEGFR3, PSMA, GPC3, IL-10RA, IL-11Rα, IL-6Rα, GP130, VEGFR2, MUC18, Met, MMP9, Thomsen-Friedenreich carbohydrate antigen, NRP-1, PDGFRβ, CD133, PTPRJ, HSPG, E-selectin, Tie2, VPAC1, ActRIIB, CD163, CXCR4, Ephrin A4, Ephrin B1, Ephrin B2, Ephrin B3, Gonadotrophin releasing hormone receptor, G Protein-coupled receptor 55, Bombesin receptor 2, IL4 receptor, Low-density lipoprotein receptor, Leptin receptor, LRP1, Melanocortin 1 receptor, Melanocortin 4 receptor, CD206, Urokinase plasminogen activator receptor, Neurokinin-1 receptor, VPAC2, ITGB1, CD27, ITGB5, ITGA1, CD27, LRP1, ACVR2B, COL13A1, NOTCH3, EGFR, VEGFR2, VEGFR3, PDGFR, HER2, ErbB3, ErbB4, RET, and FGFR102.

34. The engineered immune cell of claim 33, wherein the peptibody is an anti-IL13Rα2 peptibody, wherein the ligand polypeptide thereof comprises an amino acid sequence that is at least 90%, at least 95%, at least 96%, at least 97%, at least 98%, or at least 99% identical to SEQ ID NO: 21.

35. The engineered immune cell of claim 34, wherein the ligand polypeptide comprises one or more substitutions corresponding to positions 11, 90, 107, 103, and 108-111 of SEQ ID NO: 21.

36. The engineered immune cell of claim 35, wherein the ligand polypeptide comprises a Tyr residue at a position corresponding to Glu11 of SEQ ID NO: 21.

37. The engineered immune cell of claim 36, wherein the ligand polypeptide comprises an amino acid sequence that is at least 90%, at least 95%, at least 96%, at least 97%, at least 98%, or at least 99% identical to SEQ ID NO: 22.

38. The engineered immune cell of claim 32, wherein the ligand polypeptide comprises a ligand-mimicking peptide and/or an artificial peptide.

39. A pharmaceutical composition for treating a cancer in a subject in need thereof, comprising:
a therapeutical effective number of the engineered immune cell of any one of claims 1-38; and
a pharmaceutically acceptable adjuvant.

40. A method for treating a cancer in a subject in need thereof, comprising:
administering a therapeutically effective number of the engineered immune cell of any one of claims 1-38 to the subject.

41. A method for obtaining an autonomous growth of immune cells cultured in vitro, comprising: transducing into the immune cells a transgene encoding a chimeric polypeptide comprising an interleukin 15 (IL15) domain and an interleukin 15 receptor, alpha subunit (IL15Rα) domain, such that when expressed in the immune cells, the chimeric polypeptide is cell membrane-bound, wherein: the transduced immune cells are featured such that the total cell number thereof remains higher or substantially unchanged after at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, at least 32 days, at least 33 days, at least 34 days, or at least 35 days of culture in vitro without any supporting cytokines.

42. The method of claim 41, wherein the immune cell is a natural killer (NK) cell, a T cell, or a tumor-infiltrating cell (TIL).

43. The method of claim 42, wherein the immune cell is an NK cell.

44. The method of any one of claims 41-43, wherein the IL15 domain has an amino acid sequence that is at least 90%, at least 95%, at least 96%, at least 97%, at least 98%, or at least 99% identical to SEQ ID NO: 2.

45. The method of claim 44, wherein the IL15 domain comprises an Asp residue at a position corresponding to Asn72 of SEQ ID NO: 2.

46. The method of claim 45, wherein the IL15 domain has an amino acid sequence that is at least 90%, at least 95%, at least 96%, at least 97%, at least 98%, or at least 99% identical to SEQ ID NO: 3.

47. The method of any of claims 41-46, wherein the IL15Rα domain has an amino acid sequence that is at least 90%, at least 95%, at least 96%, at least 97%, at least 98%, or at least 99% identical to SEQ ID NO: 5.

48. The method of any of claims 41-47, wherein the IL15 domain and the IL15a domain are fused via an engineered linker, wherein the engineered linker has an amino acid sequence that is at least 90%, at least 95%, at least 96%, at least 97%, at least 98%, or at least 99% identical to SEQ ID NO: 4.

49. The method of any of claims 41-48, wherein the chimeric polypeptide has an amino acid sequence that is at least 90%, at least 95%, at least 96%, at least 97%, at least 98%, or at least 99% identical to SEQ ID NO: 6 or SEQ ID NO: 7.

50. The method of any of claims 41-49, wherein upon transduction, the total cell number of the transduced NK cells remains higher or substantially unchanged after at least 35 days of culture without any supporting cytokines.

51. The method of any of claims 42-50, wherein the NK cell is obtained from cord blood.

52. An engineered immune cell, expressing and/or secreting one or more polypeptides, each comprising a target-binding domain, wherein the target-binding domain is capable of specifically recognizing and binding to a target molecule expressed or presented on the cell surface of a target cell.

53. The engineered immune cell of claim 52, wherein the immune cell is a natural killer (NK) cell, a T cell, or a tumor-infiltrating cell (TIL).

54. The engineered immune cell of claim 53, wherein the immune cell is an NK cell.

55. The engineered immune cell of any one of claims 52-54, wherein each of the one or more polypeptides further comprises an Fc domain, wherein the Fc domain is capable of mediating antibody-dependent cellular cytotoxicity (ADCC) for the engineered immune cell.

56. The engineered immune cell of claim 55, wherein the Fc domain comprises at least one of Ser239Asp, Ala330Leu, Ile332Glu, Phe243Leu, Arg292Pro, Tyr300Leu, Val305Ile, Pro396Leu, Met428Leu, or Asn434Ser according to EU numbering.

57. The engineered immune cell of claim 56, wherein the Fc domain comprises at least one set of:
(a) Ser239Asp, Ala330Leu and Ile332Glu;
(b) Phe243Leu, Arg292Pro, Tyr300Leu, Val305Ile and Pro396Leu; or
(c) Met428Leu and Asn434Ser.

58. The engineered immune cell of claim 56 or 57, wherein the Fc domain comprises a combination of Ser239Asp, Ala330Leu, Ile332Glu, Phe243Leu, Arg292Pro, Tyr300Leu, Val305Ile, Pro396Leu, Met428Leu and Asn434Ser.

59. The engineered immune cell of any of claims 52-58, wherein each of the one or more polypeptides further comprises a first masking peptide fused at the N-terminal of the antigen-binding domain or the C-terminal of the Fc domain via a first flexible linker, wherein the first masking peptide is configured to block the Fc domain of the polypeptide, and the first flexible linker is configured to be cleavable in a target tissue.

60. The engineered immune cell of claim 59, wherein the first flexible linker is configured to be degradable by a proteinase (protease) that is specifically or enrichingly expressed in the target tissue.

61. The engineered immune cell of claim 60, wherein the proteinase is MMP1, MMP2, MMP3, MMP7, MMP9 or MMP14.

62. The engineered immune cell of claim 61, wherein the proteinase is MMP9, and the first flexible linker has an amino acid sequence selected from SEQ ID NO: 26 or SEQ ID NO: 27.

63. The engineered immune cell of claim 59, wherein the first flexible linker is configured to be degradable by a proteinase (protease) that is exogenously provided to the target tissue.

64. The engineered immune cell of any of claims 52-63, wherein each of the one or more polypeptides further comprises a second masking peptide fused at the N-terminal of the antigen-binding domain or the C-terminal of the Fc domain via a second flexible linker, wherein the second masking peptide is configured to block the antigen-binding domain of the polypeptide and the second flexible linker is configured to be cleavable in a target tissue.

65. The engineered immune cell of claim 64, wherein the second flexible linker is configured to be degradable by a proteinase (protease) that is specifically or enrichingly expressed in the target tissue.

66. The engineered immune cell of claim 65, wherein the proteinase (protease) is MMP1, MMP2, MMP3, MMP7, MMP9 or MMP14.

67. The engineered immune cell of claim 66, wherein the proteinase (protease) is MMP9, and the second flexible linker has an amino acid sequence selected from SEQ ID NO: 26 or SEQ ID NO: 27.

68. The engineered immune cell of claim 64, wherein the second flexible linker is configured to be degradable by a proteinase (protease) that is exogenously provided to the target tissue.

69. The engineered immune cell of any of claims 52-68, wherein the one or more polypeptides comprises an antibody or antigen-binding fragment thereof, wherein the target-binding domain is an antigen-binding domain, comprising a single-chain variable fragment (scFv) domain or a single monomeric variable antibody domain.

70. The engineered immune cell of claim 69, wherein the target molecule is selected from a group consisting of PD-L1, BCMA, Caudin18.2, CCR4, CD10, CD123, CD147, CD171, CD19, CD20, CD22, CD276, CD319, CD33, CD38, CD70, CLL-1, DLL3, EGFR, EGFRvIII, EpCAM, FLT3, FRα, GD2, GPC3, HER2, HGFR, IL13Ralpha2, Mesothelin, MG7, MUC1, MUC16, Nectin4, PSCA, ROR1, ROR2, TACI, TRBC1, TSLPR, and VEGFR.

71. The engineered immune cell of claim 69 or claim 70, wherein the antibody or antigen-binding fragment thereof is an anti-PD-L1 antibody or antigen-binding fragment thereof.

72. The engineered immune cell of claim 71, wherein the anti-PD-L1 antibody or antigen-binding fragment thereof comprises an scFv domain, wherein the scFv domain comprises a VL domain having an amino acid sequence that is at least 90%, at least 95%, at least 96%, at least 97%, at least 98%, or at least 99% identical to SEQ ID NO: 9, and/or comprises a VH domain having an amino acid sequence that is at least 90%, at least 95%, at least 96%, at least 97%, at least 98%, or at least 99% identical to SEQ ID NO: 10.

73. The engineered immune cell of claim 72, wherein the scFv domain of the anti-PD-L1 antibody or antigen-binding fragment thereof has an amino acid sequence that is at least 90%, at least 95%, at least 96%, at least 97%, at least 98%, or at least 99% identical to SEQ ID NO: 12.

74. The engineered immune cell of claim 71, wherein the anti-PD-L1 antibody or antigen-binding fragment thereof comprises an scFv domain, wherein the scFv domain comprises a VL domain having an amino acid sequence that is at least 90%, at least 95%, at least 96%, at least 97%, at least 98%, or at least 99% identical to SEQ ID NO: 13, and/or comprises a VH domain having an amino acid sequence that is at least 90%, at least 95%, at least 96%, at least 97%, at least 98%, or at least 99% identical to SEQ ID NO: 14.

75. The engineered immune cell of claim 74, wherein the scFv domain of the anti-PD-L1 antibody or antigen-binding fragment thereof has an amino acid sequence that is at least 90%, at least 95%, at least 96%, at least 97%, at least 98%, or at least 99% identical to SEQ ID NO: 15.

76. The engineered immune cell of any of claims 52-68, wherein the one or more polypeptides comprises a peptibody, wherein the target-binding domain comprises a ligand polypeptide.

77. The engineered immune cell of claim 76, wherein the target molecule is a receptor that the ligand polypeptide is capable of specifically recognizing and binding, wherein the receptor is selected from a group consisting of IL13Rα2, APN/CD13, APP, PD-L1, CD44, P32/gC1qR, E-cadherin, N-cadherin, CD21, EGFR, Epha2, EphB4, HER2, FGFR1, FGFR2, FGFR3, FGFR4, VEGFR1, VEGFR3, PSMA, GPC3, IL-10RA, IL-11Rα, IL-6Rα, GP130, VEGFR2, MUC18, Met, MMP9, Thomsen-Friedenreich carbohydrate antigen, NRP-1, PDGFRβ, CD133, PTPRJ, HSPG, E-selectin, Tie2, VPAC1, ActRIIB, CD163, CXCR4, Ephrin A4, Ephrin B1, Ephrin B2, Ephrin B3, Gonadotrophin releasing hormone receptor, G Protein-coupled receptor 55, Bombesin receptor 2, IL4 receptor, Low-density lipoprotein receptor, Leptin receptor, LRP1, Melanocortin 1 receptor, Melanocortin 4 receptor, CD206, Urokinase plasminogen activator receptor, Neurokinin-1 receptor, VPAC2, ITGB1, CD27, ITGB5, ITGA1, CD27, LRP1, ACVR2B, COL13A1, NOTCH3, EGFR, VEGFR2, VEGFR3, PDGFR, HER2, ErbB3, ErbB4, RET, and FGFR102.

78. The engineered immune cell of claim 77, wherein the peptibody is an anti-IL13Rα2 peptibody, wherein the ligand polypeptide thereof comprises an amino acid sequence that is at least 90%, at least 95%, at least 96%, at least 97%, at least 98%, or at least 99% identical to SEQ ID NO: 21.

79. The engineered immune cell of claim 78, wherein the ligand polypeptide comprises a one or more substitutions corresponding to positions 11, 90, 107, 103, and 108-111 of SEQ ID NO: 21.

80. The engineered immune cell of claim 79, wherein the ligand polypeptide comprises a Tyr residue at a position corresponding to Glu11 of SEQ ID NO: 21.

81. The engineered immune cell of claim 80, wherein the ligand polypeptide comprises an amino acid sequence that is at least 90%, at least 95%, at least 96%, at least 97%, at least 98%, or at least 99% identical to SEQ ID NO: 22.

82. The engineered immune cell of claim 76, wherein the ligand polypeptide comprises a ligand-mimicking peptide and/or an artificial peptide.

83. The engineered immune cell of any one of claims 53-82, wherein the engineered NK cell is capable of autonomous growth when cultured in vitro.

84. The engineered immune cell of claim 83, wherein the cell further expresses a chimeric polypeptide comprising an interleukin 15 (IL15) domain and an interleukin 15 receptor, alpha subunit (IL15Rα) domain, wherein:
when expressed, the chimeric polypeptide is cell membrane-bound and is able to operatively form a functional complex with interleukin 15 receptor, beta subunit (IL15Rβ) and interleukin 15 receptor, gamma subunit (IL15Rγ) to activate IL-15 signaling in the engineered NK cell.

85. The engineered immune cell of any of claims 53-84, wherein when a population of the NK cells are cultured in vitro, the total cell number thereof can remain substantially unchanged after at least 12 days, at least 13 days, at least 14 days, at least 15 days, at least 16 days, at least 17 days, at least 18 days, at least 19 days, at least 20 days, at least 21 days, at least 22 days, at least 23 days, at least 24 days, at least 25 days, at least 26 days, at least 27 days, at least 28 days, at least 29 days, at least 30 days, at least 31 days, or at least 32 days of culture without any supporting cytokines.

86. The engineered immune cell of any of claims 53-85, wherein the engineered NK cells are obtained from cord blood.

87. A pharmaceutical composition for treating a cancer in a subject in need thereof, comprising:
a therapeutical effective number of the engineered immune cell of any one of claims 52-86; and
a pharmaceutically acceptable adjuvant.

88. A method for treating a cancer in a subject in need thereof, comprising:
administering a therapeutically effective number of the engineered immune cell of any one of claims 52-86 to the subject.

89. An antibody or antigen-binding fragment thereof that binds to PD-L1, comprising an antigen-binding domain that specifically binds to PD-L1, wherein the antigen-binding domain comprises an scFv domain, wherein the scFv domain comprises a VL domain having an amino acid sequence that is at least 90%, at least 95%, at least 96%, at least 97%, at least 98%, or at least 99% identical to SEQ ID NO: 9, and/or comprises a VH domain having an amino acid sequence that is at least 90%, at least 95%, at least 96%, at least 97%, at least 98%, or at least 99% identical to SEQ ID NO: 10.

90. The antibody or antigen-binding fragment thereof of claim 89, wherein the scFv domain of the anti-PD-L1 antibody has an amino acid sequence that is at least 90%, at least 95%, at least 96%, at least 97%, at least 98%, or at least 99% identical to SEQ ID NO: 12.

91. An antibody or antigen-binding fragment thereof that binds to PD-L1, comprising an antigen-binding domain that specifically binds to PD-L1, wherein the antigen-binding domain comprises an scFv domain, wherein the scFv domain comprises a VL domain having an amino acid sequence that is at least 90%, at least 95%, at least 96%, at least 97%, at least 98%, or at least 99% identical to SEQ ID NO: 13, and/or comprises a VH domain having an amino acid sequence that is at least 90%, at least 95%, at least 96%, at least 97%, at least 98%, or at least 99% identical to SEQ ID NO: 14.

92. The antibody or antigen-binding fragment thereof of claim 91, wherein the scFv domain of the anti-PD-L1 antibody has an amino acid sequence that is at least 90%, at least 95%, at least 96%, at least 97%, at least 98%, or at least 99% identical to SEQ ID NO: 15.

93. The antibody or antigen-binding fragment thereof of any one of claims 89-92, further comprising an Fc domain, wherein the Fc domain is capable of mediating antibody-dependent cellular cytotoxicity (ADCC).

94. The antibody or antigen-binding fragment thereof of claim 93, wherein the Fc domain comprises at least one of Ser239Asp, Ala330Leu, Ile332Glu, Phe243Leu, Arg292Pro, Tyr300Leu, Val305Ile, Pro396Leu, Met428Leu, or Asn434Ser according to EU numbering.

95. The antibody or antigen-binding fragment thereof of claim 94, wherein the Fc domain comprises at least one set of:
(a) Ser239Asp, Ala330Leu and Ile332Glu;
(b) Phe243Leu, Arg292Pro, Tyr300Leu, Val305Ile and Pro396Leu; or
(c) Met428Leu and Asn434Ser.

96. The antibody or antigen-binding fragment thereof of claim 94 or claim 95, wherein the Fc domain comprises a combination of Ser239Asp, Ala330Leu, Ile332Glu, Phe243Leu, Arg292Pro, Tyr300Leu, Val305Ile, Pro396Leu, Met428Leu and Asn434Ser.

97. An antibody or antigen-binding fragment thereof that binds to programed death-ligand 1 (PD-L1), comprising:
a heavy chain variable region (VH) comprising complementarity determining regions (CDRs) 1, 2, and 3, wherein the VH CDR1 region comprises an amino acid sequence that is at least 80% identical to a selected VH CDR1 amino acid sequence, the VH CDR2 region comprises an amino acid sequence that is at least 80% identical to a selected VH CDR2 amino acid sequence, and the VH CDR3 region comprises an amino acid sequence that is at least 80% identical to a selected VH CDR3 amino acid sequence; and
a light chain variable region (VL) comprising CDRs 1, 2, and 3, wherein the VL CDR1 region comprises an amino acid sequence that is at least 80% identical to a selected VL CDR1 amino acid sequence, the VL CDR2 region comprises an amino acid sequence that is at least 80% identical to a selected VL CDR2 amino acid sequence, and the VL CDR3 region comprises an amino acid sequence that is at least 80% identical to a selected VL CDR3 amino acid sequence,
wherein the selected VH CDRs 1, 2, and 3 amino acid sequences and the selected VL CDRs, 1, 2, and 3 amino acid sequences are one of the following:
(1) the selected VH CDRs 1, 2, 3 amino acid sequences are set forth in SEQ ID NOs: 31, 32, 33, respectively, and the selected VL CDRs 1, 2, 3 amino acid sequences are set forth in SEQ ID NOs: 34, 35, 36, respectively;
(2) the selected VH CDRs 1, 2, 3 amino acid sequences are set forth in SEQ ID NOs: 37, 38, 39, respectively, and the selected VL CDRs 1, 2, 3 amino acid sequences are set forth in SEQ ID NOs: 40, 41, 42, respectively;
(3) the selected VH CDRs 1, 2, 3 amino acid sequences are set forth in SEQ ID NOs: 43, 44, 45, respectively, and the selected VL CDRs 1, 2, 3 amino acid sequences are set forth in SEQ ID NOs: 46, 47, 48, respectively; and
(4) the selected VH CDRs 1, 2, 3 amino acid sequences are set forth in SEQ ID NOs: 49, 50, 51, respectively, and the selected VL CDRs 1, 2, 3 amino acid sequences are set forth in SEQ ID NOs: 52, 53, 54, respectively.

98. The antibody or antigen-binding fragment thereof of claim 97, wherein the VH comprises CDRs 1, 2, 3 with the amino acid sequences set forth in SEQ ID NOs: 31, 32, and 33, respectively, and the VL comprises CDRs 1, 2, 3 with the amino acid sequences set forth in SEQ ID NOs: 34, 35, and 36, respectively according to Kabat definition.

99. The antibody or antigen-binding fragment thereof of claim 97, wherein the VH comprises CDRs 1, 2, 3 with the amino acid sequences set forth in SEQ ID NOs: 37, 38, and 39, respectively, and the VL comprises CDRs 1, 2, 3 with the amino acid sequences set forth in SEQ ID NOs: 40, 41, and 42, respectively according to Kabat definition.

100. The antibody or antigen-binding fragment thereof of claim 97, wherein the VH comprises CDRs 1, 2, 3 with the amino acid sequences set forth in SEQ ID NOs: 43, 44, and 45, respectively, and the VL comprises CDRs 1, 2, 3 with the amino acid sequences set forth in SEQ ID NOs: 46, 47, and 48, respectively according to North/Aho definition.

101. The antibody or antigen-binding fragment thereof of claim 97, wherein the VH comprises CDRs 1, 2, 3 with the amino acid sequences set forth in SEQ ID NOs: 49, 50, and 51, respectively, and the VL comprises CDRs 1, 2, 3 with the amino acid sequences set forth in SEQ ID NOs: 52, 53, and 54, respectively according to North/Aho definition.

102. The antibody or antigen-binding fragment thereof of any one of claims 97-101, wherein the antibody or antigen-binding fragment specifically binds to human PD-L1.

103. The antibody or antigen-binding fragment thereof of any one of claims 97-102, wherein the antibody or antigen-binding fragment is a human or humanized antibody or antigen-binding fragment thereof.

104. The antibody or antigen-binding fragment thereof of any one of claims 97-103, wherein the antibody or antigen-binding fragment is a single-chain variable fragment (scFv) or a multi-specific antibody (e.g., a bispecific antibody).

105. A nucleic acid comprising a polynucleotide encoding a polypeptide comprising:
(1) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 31, 32, and 33, respectively (or SEQ ID NOs: 43, 44, and 45, respectively), and wherein the VH, when paired with a light chain variable region (VL) comprising the amino acid sequence set forth in SEQ ID NO: 13 binds to PD-L1;
(2) an immunoglobulin light chain or a fragment thereof comprising a VL comprising CDRs 1, 2, and 3 comprising the amino acid sequences set forth in SEQ ID NOs: 34, 35, and 36, respectively (or SEQ ID NOs: 46, 47, and 48, respectively), and wherein the VL, when paired with a VH comprising the amino acid sequence set forth in SEQ ID NO: 14 binds to PD-L1;
(3) an immunoglobulin heavy chain or a fragment thereof comprising a heavy chain variable region (VH) comprising complementarity determining regions (CDRs) 1, 2, and 3 comprising the amino acid sequences set forth in SEQ ID NOs: 37, 38, and 39, respectively (or SEQ ID NOs: 49, 50, and 51, respectively), and wherein the VH, when paired with a light chain variable region (VL) comprising the amino acid sequence set forth in SEQ ID NO: 9 binds to PD-L1; or
(4) an immunoglobulin light chain or a fragment thereof comprising a VL comprising CDRs 1, 2, and 3 comprising the amino acid sequences set forth in SEQ ID NOs: 40, 41, and 42, respectively (or SEQ ID NOs: 52, 53, and 54, respectively), and wherein the VL, when paired with a VH comprising the amino acid sequence set forth in SEQ ID NO: 10 binds to PD-L1.

106. The nucleic acid of claim 105, wherein the VH when paired with a VL specifically binds to human PD-L1, or the VL when paired with a VH specifically binds to human PD-L1.

107. The nucleic acid of claim 105 or 106, wherein the immunoglobulin heavy chain or the fragment thereof is a human or humanized immunoglobulin heavy chain or a fragment thereof, and the immunoglobulin light chain or the fragment thereof is a human or humanized immunoglobulin light chain or a fragment thereof.

108. The nucleic acid of any one of claims 105-107, wherein the nucleic acid encodes a single-chain variable fragment (scFv), a multi-specific antibody (e.g., a bispecific antibody), or a chimeric antigen receptor (CAR).

109. The nucleic acid of any one of claims 105-108, wherein the nucleic acid is cDNA.

110. A vector comprising one or more of the nucleic acids of any one of claims 105-109.

111. A vector comprising two of the nucleic acids of any one of claims 105-109, wherein the vector encodes the VH region and the VL region that together bind to PD-L1.

112. A pair of vectors, wherein each vector comprises one of the nucleic acids of any one of claims 105-109, wherein together the pair of vectors encodes the VH region and the VL region that together bind to PD-L1.

113. A cell comprising the vector of claim 110 or 111, or the pair of vectors of claim 112.

114. The cell of claim 113, wherein the cell is a CHO cell.

115. A cell comprising one or more of the nucleic acids of any one of claims 105-109.

116. A method of producing an antibody or an antigen-binding fragment thereof, the method comprising
(a) culturing the cell of any one of claims 113-115 under conditions sufficient for the cell to produce the antibody or the antigen-binding fragment; and
(b) collecting the antibody or the antigen-binding fragment produced by the cell.

117. An antibody or antigen-binding fragment thereof that binds to PD-L1 comprising
a heavy chain variable region (VH) comprising an amino acid sequence that is at least 80% identical to a selected VH sequence, and a light chain variable region (VL) comprising an amino acid sequence that is at least 80% identical to a selected VL sequence, wherein the selected VH sequence and the selected VL sequence are one of the following:
(1) the selected VH sequence is SEQ ID NO: 14, and the selected VL sequence is SEQ ID NO: 13; and
(2) the selected VH sequence is SEQ ID NO: 10, and the selected VL sequence is SEQ ID NO: 9.

118. The antibody or antigen-binding fragment thereof of claim 117, wherein the antibody or antigen-binding fragment specifically binds to human PD-L1.

119. The antibody or antigen-binding fragment thereof of claim 117 or 118, wherein the antibody or antigen-binding fragment is a human or humanized antibody or antigen-binding fragment thereof.

120. The antibody or antigen-binding fragment thereof of any one of claims 117-119, wherein the antibody or antigen-binding fragment is a single-chain variable fragment (scFv) or a multi-specific antibody (e.g., a bispecific antibody).

121. An antibody or antigen-binding fragment thereof that cross-competes with the antibody or antigen-binding fragment thereof of any one of claims 97-104 and 117-120.

122. An antibody or antigen-binding fragment thereof that binds to PD-L1 comprising
a heavy chain variable region (VH) comprising VH CDR1, VH CDR2, and VH CDR3 that are identical to VH CDR1, VH CDR2, and VH CDR3 of a selected VH sequence; and
a light chain variable region (VL) comprising VL CDR1, VL CDR2, and VL CDR3 that are identical to VL CDR1, VL CDR2, and VL CDR3 of a selected VL sequence, wherein the selected VH sequence and the selected VL sequence are one of the following:
(1) the selected VH sequence is SEQ ID NO: 14, and the selected VL sequence is SEQ ID NO: 13; and
(2) the selected VH sequence is SEQ ID NO: 10, and the selected VL sequence is SEQ ID NO: 9.

123. An antibody-drug conjugate comprising the antibody or antigen-binding fragment thereof of any one of claims 97-104 and 117-122 covalently bound to a therapeutic agent.

124. The antibody drug conjugate of claim 123, wherein the therapeutic agent is a cytotoxic or cytostatic agent.

125. A method of treating a subject having cancer, the method comprising administering a therapeutically effective amount of a composition comprising the antibody or antigen-binding fragment thereof of any one of claims 97-104 and 117-122, or the antibody-drug conjugate of claim 123 or 124, to the subject.

126. The method of claim 51, wherein the subject has breast cancer, ovarian cancer, lung cancer, bladder cancer, prostate cancer, pancreatic cancer, gastric cancer, liver cancer, leukemia and/or lymphoma.

127. A method of decreasing the rate of tumor growth, the method comprising contacting a tumor cell with an effective amount of a composition comprising an antibody or antigen-binding fragment thereof of any one of claims 97-104 and 117-122, or the antibody-drug conjugate of claim 123 or 124.

128. A method of killing a tumor cell, the method comprising contacting a tumor cell with an effective amount of a composition comprising the antibody or antigen-binding fragment thereof of any one of claims 97-104 and 117-122, or the antibody-drug conjugate of claim 123 or 124.

129. A pharmaceutical composition comprising the antibody or antigen-binding fragment thereof of any one of claims 97-104 and 117-122, and a pharmaceutically acceptable carrier.

130. A pharmaceutical composition comprising the antibody drug conjugate of claim 123 or 124, and a pharmaceutically acceptable carrier.


SEQUENCE LISTING (54 sequences)

SEQ ID NO: 1 - protein, 35 aa, Homo sapiens
MAPRRARGCRTLGLPALLLLLLLRPPATRGITAPP

SEQ ID NO: 2 - protein, 114 aa, Homo sapiens
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 114 aa, synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 4 - protein, 33 aa, synthetic construct
GSGGSGGGSGGGGSGGGGSGGGGSGGGSGSLQI

SEQ ID NO: 5 - protein, 236 aa, Homo sapiens
TCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 6 - protein, 418 aa, synthetic construct
MAPRRARGCRTLGLPALLLLLLLRPPATRGITAPPNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGGSGGGSGGGGSGGGGSGGGGSGGGSGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 7 - protein, 383 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSGSGGSGGGSGGGGSGGGGSGGGGSGGGSGSLQITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 8 - protein, 20 aa, Homo sapiens
MYRMQLLSCIALSLALVTNS

SEQ ID NO: 9 - protein, 106 aa, synthetic construct
AIQLTQSPSSLSASVGDRVTITCSASSSVSYMHWYQQKPGKAPKLWIYDTSKLASGVPSRFSGSGSGTDFTLTISSLQPEDFATYYCFQESGYPLTFGGGTKLTVL

SEQ ID NO: 10 - protein, 119 aa, synthetic construct
QVQLQESGPGLVKPSETLSLTCTVSGDSITSGYWNWIRQPPGKGLEYMGYISFTGSTYYNPSLKSRVTISVDTSKNQFSLKLSSVTAADTAVYYCARKREWILHPMDYWGQGTLVTVSS

SEQ ID NO: 11 - protein, 15 aa, synthetic construct
GGGGSGGGGSGGGGS

SEQ ID NO: 12 - protein, 240 aa, synthetic construct
AIQLTQSPSSLSASVGDRVTITCSASSSVSYMHWYQQKPGKAPKLWIYDTSKLASGVPSRFSGSGSGTDFTLTISSLQPEDFATYYCFQESGYPLTFGGGTKLTVLGGGGSGGGGSGGGGSQVQLQESGPGLVKPSETLSLTCTVSGDSITSGYWNWIRQPPGKGLEYMGYISFTGSTYYNPSLKSRVTISVDTSKNQFSLKLSSVTAADTAVYYCARKREWILHPMDYWGQGTLVTVSS

SEQ ID NO: 13 - protein, 106 aa, synthetic construct
ENVLTQSPAIMSASPGEKVTMTCSASSSVSYMHWYQQKSLTSPKLWIYDTSKLASGVPGRFSGSGSGNFYSLTISSMEAEDVATYYCFQESGYPLTFGAGTKLELK

SEQ ID NO: 14 - protein, 119 aa, synthetic construct
EVQLQESGPSLVKPSQTLSLTCSVTGDSITSGYWNWIRKFPENKLEYMGYISFTGSTYYNPSLKSRISITRDTSKNQYYLQLNSVTTEDTATYYCARKREWILHPMDYWGQGTSVTVSS

SEQ ID NO: 15 - protein, 240 aa, synthetic construct
ENVLTQSPAIMSASPGEKVTMTCSASSSVSYMHWYQQKSLTSPKLWIYDTSKLASGVPGRFSGSGSGNFYSLTISSMEAEDVATYYCFQESGYPLTFGAGTKLELKGGGGSGGGGSGGGGSEVQLQESGPSLVKPSQTLSLTCSVTGDSITSGYWNWIRKFPENKLEYMGYISFTGSTYYNPSLKSRISITRDTSKNQYYLQLNSVTTEDTATYYCARKREWILHPMDYWGQGTSVTVSS

SEQ ID NO: 16 - protein, 12 aa, Homo sapiens
EPKSCDKTHTCP

SEQ ID NO: 17 - protein, 113 aa, Homo sapiens
PCPAPELLAGPDVFLLPPKPKDTLMISRTPEVTCVVVDVSFEDPEVKFNWYVDGVEVHNAKTKPPEEQYNSTLRVVSILTVLHQDWLNGKEYKCKVTNKALPLPEEKTISKAK

SEQ ID NO: 18 - protein, 107 aa, Homo sapiens
GQPREPQVYTLPPSRDELTKNQVSLTCLVKGFYPSDIAVEWESNGQPENNYKTTPLVLDSDGSFFLYSKLTVDKSRWQQGNVFSCSVLHEALHSHYTQKSLSLSPGK

SEQ ID NO: 19 - protein, 492 aa, synthetic construct
MYRMQLLSCIALSLALVTNSAIQLTQSPSSLSASVGDRVTITCSASSSVSYMHWYQQKPGKAPKLWIYDTSKLASGVPSRFSGSGSGTDFTLTISSLQPEDFATYYCFQESGYPLTFGGGTKLTVLGGGGSGGGGSGGGGSQVQLQESGPGLVKPSETLSLTCTVSGDSITSGYWNWIRQPPGKGLEYMGYISFTGSTYYNPSLKSRVTISVDTSKNQFSLKLSSVTAADTAVYYCARKREWILHPMDYWGQGTLVTVSSEPKSCDKTHTCPPCPAPELLAGPDVFLLPPKPKDTLMISRTPEVTCVVVDVSFEDPEVKFNWYVDGVEVHNAKTKPPEEQYNSTLRVVSILTVLHQDWLNGKEYKCKVTNKALPLPEEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVLHEALHSHYTQKSLSLSPGK

SEQ ID NO: 20 - protein, 492 aa, synthetic construct
MYRMQLLSCIALSLALVTNSENVLTQSPAIMSASPGEKVTMTCSASSSVSYMHWYQQKSLTSPKLWIYDTSKLASGVPGRFSGSGSGNFYSLTISSMEAEDVATYYCFQESGYPLTFGAGTKLELKGGGGSGGGGSGGGGSEVQLQESGPSLVKPSQTLSLTCSVTGDSITSGYWNWIRKFPENKLEYMGYISFTGSTYYNPSLKSRISITRDTSKNQYYLQLNSVTTEDTATYYCARKREWILHPMDYWGQGTSVTVSSEPKSCDKTHTCPPCPAPELLAGPDVFLLPPKPKDTLMISRTPEVTCVVVDVSFEDPEVKFNWYVDGVEVHNAKTKPPEEQYNSTLRVVSILTVLHQDWLNGKEYKCKVTNKALPLPEEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVLHEALHSHYTQKSLSLSPGK

SEQ ID NO: 21 - protein, 112 aa, Homo sapiens
GPVPPSTALRELIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 22 - protein, 112 aa, synthetic construct
GPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFN

SEQ ID NO: 23 - protein, 23 aa, synthetic construct
GGSAEAAAKEAAAKEAAAKAGSS

SEQ ID NO: 24 - protein, 220 aa, Homo sapiens
PCPAPELLAGPDVFLLPPKPKDTLMISRTPEVTCVVVDVSFEDPEVKFNWYVDGVEVHNAKTKPPEEQYNSTLRVVSILTVLHQDWLNGKEYKCKVTNKALPLPEEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVLHEALHSHYTQKSLSLSPGK

SEQ ID NO: 25 - protein, 364 aa, synthetic construct
MYRMQLLSCIALSLALVTNSGPVPPSTALRYLIEELVNITQNQKAPLCNGSMVWSINLTAGMYCAALESLINVSGCSAIEKTQRMLSGFCPHKVSAGQFSSLHVRDTKIEVAQFVKDLLLHLKKLFREGRFNEPKSCDKTHTCPPCPAPELLAGPDVFLLPPKPKDTLMISRTPEVTCVVVDVSFEDPEVKFNWYVDGVEVHNAKTKPPEEQYNSTLRVVSILTVLHQDWLNGKEYKCKVTNKALPLPEEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVLHEALHSHYTQKSLSLSPGK

SEQ ID NO: 26 - protein, 7 aa, synthetic construct
VPLSLYS

SEQ ID NO: 27 - protein, 6 aa, synthetic construct
SPLGLA

SEQ ID NO: 28 - protein, 12 aa, synthetic construct
GGGSGGGSGGGS

SEQ ID NO: 29 - protein, 5 aa, synthetic construct
GGGGS

SEQ ID NO: 30 - protein, 4 aa, synthetic construct
GGGS

SEQ ID NO: 31 - protein, 5 aa, synthetic construct
SGYWN

SEQ ID NO: 32 - protein, 16 aa, synthetic construct
YISFTGSTYYNPSLKS

SEQ ID NO: 33 - protein, 11 aa, synthetic construct
KREWILHPMDY

SEQ ID NO: 34 - protein, 10 aa, synthetic construct
SASSSVSYMH

SEQ ID NO: 35 - protein, 7 aa, synthetic construct
DTSKLAS

SEQ ID NO: 36 - protein, 9 aa, synthetic construct
FQESGYPLT

SEQ ID NO: 37 - protein, 5 aa, synthetic construct
SGYWN

SEQ ID NO: 38 - protein, 16 aa, synthetic construct
YISFTGSTYYNPSLKS

SEQ ID NO: 39 - protein, 11 aa, synthetic construct
KREWILHPMDY

SEQ ID NO: 40 - protein, 10 aa, synthetic construct
SASSSVSYMH

SEQ ID NO: 41 - protein, 7 aa, synthetic construct
DTSKLAS

SEQ ID NO: 42 - protein, 9 aa, synthetic construct
FQESGYPLT

SEQ ID NO: 43 - protein, 13 aa, synthetic construct
SVTGDSITSGYWN

SEQ ID NO: 44 - protein, 9 aa, synthetic construct
YISFTGSTY

SEQ ID NO: 45 - protein, 13 aa, synthetic construct
ARKREWILHPMDY

SEQ ID NO: 46 - protein, 10 aa, synthetic construct
SASSSVSYMH

SEQ ID NO: 47 - protein, 8 aa, synthetic construct
YDTSKLAS

SEQ ID NO: 48 - protein, 9 aa, synthetic construct
FQESGYPLT

SEQ ID NO: 49 - protein, 13 aa, synthetic construct
TVSGDSITSGYWN

SEQ ID NO: 50 - protein, 9 aa, synthetic construct
YISFTGSTY

SEQ ID NO: 51 - protein, 13 aa, synthetic construct
ARKREWILHPMDY

SEQ ID NO: 52 - protein, 10 aa, synthetic construct
SASSSVSYMH

SEQ ID NO: 53 - protein, 8 aa, synthetic construct
YDTSKLAS

SEQ ID NO: 54 - protein, 9 aa, synthetic construct
FQESGYPLT